(19) United States
(12) Patent Application Publication
(10) Pub. No.: US 2023/0203130 A1
(43) Pub. Date: Jun. 29, 2023

(54) A PEPTIDE COCKTAIL

(71) Applicant: Hubro Therapeutics AS, Oslo (NO)

(72) Inventors: Henrik ERIKSEN, Oslo (NO); Jon Amund ERIKSEN, Oslo (NO)

(73) Assignee: Hubro Therapeutics AS, Oslo (NO)

(21) Appl. No.: 17/927,544

(22) PCT Filed: May 28, 2021
(86) PCT No.: PCT/EP2021/064415

§ 371 Date: Nov. 23, 2022

(30) Foreign Application Priority Data
May 28, 2020 (EP) 20177183.9

(51) Int. Cl. C07K 14/71; A61P 35/00; C12N 15/63

(52) U.S. Cl. CPC C07K 14/71; A61P 35/00; C12N 15/63

(57) ABSTRACT

There is disclosed a peptide capable of inducing an immune response against: a ASTE1-1a frameshift mutant protein, wherein the peptide comprises at least 10 consecutive amino acids of SEQ ID NO: 26; a TAE1β-1a frameshift mutant protein, wherein the peptide comprises at least 10 consecutive amino acids of SEQ ID NO: 27; a KIAA2018-1a frameshift mutant protein, wherein the peptide comprises a immunogenic fragment of one of SEQ ID NOs: 9-12, wherein the fragment comprises at least 8 consecutive amino acids of one of one of SEQ ID NOs: 9-12; or a SLC22A9-1a frameshift mutant protein, wherein the peptide comprises at least 8 consecutive amino acids of one of SEQ ID NOs: 14-18. There is also disclosed a peptide mixture comprising a first and second peptide, each independently selected from one of the peptides described above and a peptide capable of inducing an immune response against a TΘEβE2-1a frameshift mutant protein.

### FIELD OF INVENTION

The present invention provides peptides of ASTE1 Having a frameshift mutation, TAF1β having a frameshift mutation, KIAA2018 having a frameshift mutation and SLC22A9 having a frameshift mutation, capable of eliciting an immune response. The present invention also provides peptide mixtures comprising a first and second peptide, each independently selected from the peptides of the invention and peptides of TGBβR2 having a frameshift mutation capable of eliciting an immune response. The present invention also provides T-cell receptors and T-cells specific for such peptides, and T-cell mixtures and T-cell preparations comprising T-cells specific for such peptides and peptide mixtures, nucleic acid molecules encoding one or more of the peptides, vectors comprising the nucleic acid molecule and host cells comprising the vector. The present invention further provides pharmaceutical formulations comprising such peptides, peptide mixtures, T-cells and T-cell mixtures and preparations, uses of such peptides, peptide mixtures, T-cell receptors, T-cells and T-cell mixtures and preparations for the prophylaxis and/or treatment of cancer, and methods of selecting peptides, peptide mixtures, T-cell receptors, T-cells, T-cell mixtures and T-cell preparations for the treatment of cancer.

### BACKGROUND

DNA microsatellites are strings of repetitive DNA, in which certain DNA motifs (nucleotide sequence patterns) are repeated, usually about 5 to 50 times. Microsatellite instability (MSI) is a change in the number of repeats of microsatellites and can be caused by impaired DNA mismatch repair (MMR) enzyme activity.

MMR corrects errors that occur spontaneously during DNA replication, such as single base mismatches or short insertions or deletions. When MMR activity is impaired, these spontaneous errors are not repaired, and this can result in microsatellite instability (i.e. a change in the number of repeats) and frameshift mutations in the DNA microsatellite sequences.

Frameshift mutations are the addition or deletion of one or two base pairs from a gene, resulting in different codons, and, therefore, a different protein being encoded, from the point of mutation. The frameshift typically results in truncated protein sequences because a STOP codon occurs prematurely, and the encoded proteins are usually defective or inactive.

In recent years, immuno-oncology has been a developing field, with efforts focussed on using the patient's own immune system to fight cancer. However, one problem is that antibodies can only bind to tumour antigens that are exposed on the surface of tumour cells. For this reason the efforts to produce a cancer treatment based on the immune system of the body has been less successful than expected.

Several proteins have been identified as frequently having frameshift mutations in MSI-H cancers. For example, TGFβR2 (SEQ ID NO: 1) is a growth factor, and its interaction with TGFβ mediates control of cell growth. Frameshift mutations in TGFβR2 render it biologically non-functional, thereby inducing uncontrolled cell growth and cancer progression. Frameshift mutations are also found in ASTE1 (SEQ ID NO: 4), TAF1p (SEQ ID NO: 6), KIAA2018 (SEQ ID NO: 8) and SLC22A9 (SEQ ID NO: 13), and single nucleotide deletions in the genes encoding these proteins are by far the most dominant frameshift mutation in these proteins, although it is possible for a single nucleotide addition to occur. In particular, it has been found that more than 95% of mutations in microsatellites are deletions (Maby et al., Cancer Res, 75(17), Sep. 1, 2015). The amino acid sequence of each of TGFβR2, ASTE1, TAF13, KIAA2018 and SLCC22A resulting from a single nucleotide deletion (−1a) frameshift mutation is shown in SEQ ID NO: 2 (TGFβR2), SEQ ID NO: 5 (ASTE1), SEQ ID NO: 7 (TAF1p), SEQ ID NOs: 9-12 (KIAA2018, which can in fact have a nucleotide deletion at any of four positions), and SEQ ID NOs: 14-16 (SLC22A9, which can in fact have a nucleotide deletion at any of three positions).

The detection of MSI in cancer, such as colorectal cancers (CRCs), is performed by profiling the Bethesda panel, which is a reference panel including five microsatellite loci: two mononucleotides (BAT25 and BAT26) and three dinucleotides (D5S346, D2S123 and D17S250) (Cortes-Ciriano et al., Nature Communications, 2017, vol. 8, article no. 15180; Vilar & Gruber, Nat Rev Clin Oncol, 2010, vol. 7(3), p. 153-162). MSI is classed as high (MSI-H) when there is instability at two or more loci, and is classed as low (MSI-L) when there is instability at one locus (Vilar & Gruber, Nat Rev Clin Oncol, 2010, vol. 7(3), p. 153-162). Microsatellites can be classed as stable (MSS) when there is no loci which has instabilities (Vilar & Gruber, Nat Rev Clin Oncol, 2010, vol. 7(3), p. 153-162).

About 15% of all CRCs are MSI-H, and MSI has also been reported in glioblastomas, lymphomas, stomach, urinary tract, ovarian and endometrial tumours (Cortes-Ciriano et al., Nature Communications, 2017, vol. 8, article no. 15180). It has also been reported that each of TAF1β, ASTE1 and TGFβR2 is independently mutated in more than 75% of MSI CRCs (Maby et al., Cancer Res, 75(17), Sep. 1, 2015).

In addition, 99% of hereditary CRCs (Hereditary Non-Polyposis Colorectal Cancer (HNPCC)) are MSI-H (Pinheiro el al., British Journal of Cancer, 2015, vol. 113, p. 686-692). Of the MSI-H HNPCC patients, about 90% have a frameshift mutation in the protein TGFβR2 (Pinheiro el al., British Journal of Cancer, 2015, vol. 113, p. 686-692).

People with HNPCC have a somatic mutation which is expected to develop into a frameshift mutation. CRC is often preceded by the development of polyps, but the removal of these from patients with hereditary CRC is ineffective in preventing cancer, unlike in patients who do not have hereditary CRC.

In addition, about 22% of stomach (gastric) cancers are MSI-H (Cortes-Ciriano et al., Nature Communications, 2017, vol. 8, article no. 15180).

Furthermore, frameshift mutations in TGFβR2 are reported to be found in about 15% of all CRCs, about 44% of all MSI-H cancers, and in particular in about 58% of MSI-H colon cancers and about 80% of MSI-H stomach cancers (Cortes-Ciriano et al., Nature Communications, 2017, vol. 8, article no. 15180). Frameshift mutations in KIAA2018, SLC22A9 and ASTE1 are found in about 51%, 50% and 45%, respectively, of all MSI-H cancers (Cortes-Ciriano et al., Nature Communications, 2017, vol. 8, article no. 15180).

Peptides of TGFβR2 having a frameshift mutation have been reported to be immunogenic, although there are inconsistencies in the results reported, as discussed below.

EP1078000 discloses using fragments of proteins arising from frameshift mutations in the BAX and TGFβR2 genes to treat cancer, by eliciting T-cell immunity. Linnebacher et al. (Int J Cancer, 2001, 93, p. 6-11) reports that three peptides derived from proteins having frameshift mutations were capable of activating specific CTLs (HLA-A2.1 restricted) in vitro, including a peptide (referred to therein as FSP02: RLSSCVPVA; SEQ ID NO: 33) of TGFβR2 having a −1a frameshift mutation. This peptide was also able to lyse the colorectal cancer cell line HCT116, which carries the corresponding frameshift mutation. However, two other peptides of −1a frameshifted TGFβR2 did not activate CTLs.

Saeterdal et al. (Cancer Immunol Immunother, 2001 November, 50(9), 469-476) reports that a peptide (RLSSCVPVA (labelled in Saeterdal et al., Cancer Immunol Immunother as p573); SEQ ID NO: 33 herein) of TGFβR2 having a frameshift mutation was able to generate a CTL line and several CTL clones. One CTL clone was able to kill an HLA-A2+ colon cancer cell line harbouring the frameshifted TGFβR2.

Saeterdal et al. (PNAS, 2001 Nov. 6, 98(23), 13255-13260) reports a highly immunogenic peptide (labelled p538; SLVRLSSCVPVALMSAMTTSSSQ (SEQ ID NO: 34 herein)) derived from TGFβR2 having a frameshift mutation, as a target for tumour infiltrating Th cells in MSI+ tumours. This peptide was recognized by two of three patients having spontaneous MSI+ colon cancer, and from all three patients with HNPCC. Several other peptides corresponding to the same frameshift mutation (p540: SPKCIMKEKKSLVRLSSCVPVA (SEQ ID NO: 35 herein), and p541: PKCIMKEKKKSLVRLSSCV (SEQ ID NO: 36 herein)) were also able to induce T-cell responses in some patients.

However, the studies above have contradictory results, such that it is not clear whether or not the peptides of TGFβR2 having a frameshift mutation are indeed immunogenic. For example, peptide FSP01 (SLVRLSSCV; SEQ ID NO: 37 herein) of Linnebacher et al. is the same as peptide SEQ ID NO: 428 of EP1078000, but Linnebacher et al. reports that this peptide is not immunogenic (FIG. 1 of Linnebacher et al.) while EP1078000 reports that this peptide is immunogenic (FIG. 14 of EP1078000), albeit only after four rounds of stimulation of the T-cells. Peptide FSP02 (RLSSCVPVA; SEQ ID NO: 33 herein) of Linnebacher et al. is the same as peptide SEQ ID NO: 439 of EP1078000 and peptide p573 of Saeterdal et al., (2001, Cancer Immunol Immunother), but Linnebacher et al. and Saeterdal et al (2001, Cancer Immunol Immunother) report that this peptide is immunogenic (FIG. 1 of Linnebacher et al.; abstract, and page 472, column 1, paragraph 3, of Saeterdal et al.), while EP 1078000 reports that this peptide is not immunogenic even after four rounds of T-cell stimulation (FIG. 14 of EP 1078000). Moreover, Saeterdal (2001, PNAS) discloses that the peptides p537 and p621 are not immunogenic but that the peptides p538, p540 and p4541 are immunogenic; however, both p538 and p621 comprise the sequence of peptides p523 and p573 which have been shown to be immunogenic by some studies (as discussed above). Thus, it is unclear why p621 is not immunogenic while p538 is immunogenic. Furthermore, EP1078000 discloses that the peptide SEQ ID NO: 17 thereof is capable of stimulating cultivated T-cell clones derived from a patient with adenocarcinoma (FIGS. 8 and 9 of EP1078000), but that the peptide SEQ ID NO: 17 thereof does not induce a T-cell response above background values in T-cells from healthy blood donors (FIG. 12 of EP1078000). The results shown in FIGS. 8 and 9 of EP1078000 show that a spontaneous T-cell immune response might have been induced in a patient with cancer and that these T-cells, after cultivation with peptide SEQ ID NO: 17, can recognise peptide SEQ ID NO:17. However, these results do not show that the peptide SEQ ID NO: 17 is a strong enough antigen to induce a protective immune response.

It has also been found that, using engineered antigen-presenting cells, it was possible to stimulate peripheral cytotoxic T-cells obtained from colorectal cancer patients with peptides derived from frameshift mutations found in the patients' tumours (Maby et al., Cancer Res, 75(17), Sep. 1, 2015). In particular, the peptides were derived from the frameshift mutants of TGFβR2, TAF1β and ASTE1. However, T-cells from cancer patients without frameshift mutations, and T-cells from healthy subjects, could not be stimulated to detectable levels, which indicates that the peptides are not sufficiently immunogenic to induce a detectable immune response if used for vaccination.

U.S. Pat. No. 8,053,552 discloses that peptides derived from −1a frameshifted TGFβR2, TAF1β and ASTE1 were able, in vitro, to induce an immune response using T-cells from healthy HLA-A2.1+ donors. However, these results are limited only to HLA-A2.1+ epitopes, and do not show that other HLA class I-restricted T-cells, or any HLA class 11-restricted T-cells, were induced. Historically, vaccines consisting only of HLA class I epitopes have not been successful in treating cancer and, therefore, U.S. Pat. No. 8,053,552 does not show that the peptides tested therein are an effective vaccine or treatment for cancer.

WO2014/090265 and EP2572725 disclose a vaccine for the treatment or prevention of cancer, which comprises a MSI-specific frameshift peptide or mixture of such peptides. The frameshift peptide is derived from TAF1β, HT001 (also known as ASTE1), TGFβR2 or AIM2. WO2014/090265 and EP2572725 disclose that one specific frameshift peptides derived from each TAF1β, HT001, TGFβR2 and AIM2 were, individually, able to induce a T-cell response in vitro.

WO96/31605 discloses a mutant protein of TGFβR2 which has a −1a frameshift mutation, and a method of treating cancer comprising administering to a patient a composition comprising non-functional TGFβR2.

WO2018/223093 discloses a method of treating or preventing cancer by administering to a patient at least one peptide comprising a frameshift variant in a microsatellite coding region of an expressed gene, which may be a deletion or an addition. This document discloses several specific TAF1β, ASTE1 and TGFβR2 frameshift peptides, but none have been tested for immunogenicity in humans.

WO2020/239937 discloses peptides of frameshifted TGFβR2 and their use for the treatment or prevention of cancer.

Thus, there is a need to provide effective vaccines and/or treatments for cancers, particularly cancers associated with MSI and frameshift mutations. In particular, there is a need to provide vaccines and/or treatments for these cancers which are cost effective and can be used to treat or vaccine against as many MSI-H-associated cancers as possible.

### SUMMARY OF INVENTION

The present invention alleviates at least some of the problems above because it has now been found that peptides comprising a fragment of ASTE1, TAF1β, KIAA2018 or SLC22A9 having a frameshift mutation can be used to induce an immune response against cancer cells and, therefore, are useful for the treatment and/or prophylaxis of cancer associated with ASTE1, TAF1β, KIAA2018 and/or SLC22A9 having a frameshift mutation. In addition, it has been found that a peptide mixture comprising a first and a second peptide, each independently selected from these peptides and a peptide comprising a fragment of TGFβR2 having a frameshift mutation, can be used to induce an immune response against cancer cells. Thus, the peptide mixtures are useful for the treatment and/or prophylaxis of cancer associated with frameshift mutations in one or more of TGFβR2, ASTE1, TAF1β, KIAA2018 and SLC22A9. The peptides and peptide mixtures of the present invention are particularly useful for the treatment and/or prophylaxis of cancers associated with −1a frameshift mutations in one or more of TGFβR2, ASTE1, TAF1β, KIAA2018 and SLC22A9. It has been found that particularly useful peptides comprise a fragment which corresponds to at least part of the mutated amino acid sequence resulting from the frameshift mutation in the relevant protein. In addition, particularly useful peptides are derived from a protein which has a frameshift mutation in the patient in question. In particular, the peptides and peptide mixtures may be used to treat all MSI-H colorectal cancers, and a large proportion of all MSI-H cancers. The peptides of the invention comprise multiple nested epitopes, such that the peptides comprise epitopes for more than one HLA allele. This provides the advantage that the peptides are capable of inducing an immune response in patients having different HLA alleles, such that the peptides are useful as a universal treatment and/or vaccine. In addition, each peptide of the invention contain few or no amino acid residues from the wild-type amino acid sequence of the protein that each peptide is derived from, thereby reducing the risk that the peptides of the invention induce an autoimmune response. Moreover, the peptide mixtures of the invention provide an effective and cost-effective vaccine and/or treatment because it provides a treatment and/or vaccine for a large proportion of all MSI-H cancers and, more particularly, can provide a treatment for all MSI-H colorectal cancers.

In a first aspect of the invention, there is provided a peptide mixture comprising a first and a second peptide, each independently selected from:
a peptide capable of inducing an immune response against a TGFβR2-1a frameshift mutant protein, wherein the peptide comprises i) an immunogenic fragment of SEQ ID NO: 3, wherein the fragment comprises at least 8 consecutive amino acids of SEQ ID NO: 3 including at least one of positions 121 and 135 of SEQ ID NO: 3, or ii) an immunogenic fragment of SEQ ID NO: 2, wherein the fragment comprises at least 8 consecutive amino acids of SEQ ID NO: 19;
a peptide capable of inducing an immune response against a ASTE1-1a frameshift mutant protein, wherein the peptide comprises an immunogenic fragment of SEQ ID NO: 5, wherein the fragment comprises at least 8 consecutive amino acids of SEQ ID NO: 26;
a peptide capable of inducing an immune response against a TAF1β-1a frameshift mutant protein, wherein the peptide comprises an immunogenic fragment of SEQ ID NO: 7, wherein the fragment comprises at least 8 consecutive amino acids of SEQ ID NO: 27;
a peptide capable of inducing an immune response against a KIAA2018-1a frameshift mutant protein, wherein the peptide comprises a immunogenic fragment of one of SEQ ID NOs: 9-12, wherein the fragment comprises at least 8 consecutive amino acids of one of one of SEQ ID NOs: 9-12, respectively, including at least one amino acid from positions 13 to 37 of SEQ ID NO: 9, positions 91 to 109 of SEQ ID NO: 10, positions 147 to 167 of SEQ ID NO: 11 and positions 1016 to 1037 of SEQ ID NO: 12, respectively; and
a peptide capable of inducing an immune response against a SLC22A9-1a frameshift mutant protein, wherein the peptide comprises an immunogenic fragment of one of SEQ ID NO: 14-18, wherein the fragment comprises at least 8 consecutive amino acids of one of SEQ ID NOs: 14-18, respectively, including at least one amino acid from positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18, respectively;
wherein the first peptide is capable of inducing an immune response against a different frameshift mutant protein from the second peptide.

Preferably, the peptide comprising an immunogenic fragment of SEQ ID NO: 3 comprises at least 9 consecutive amino acids of SEQ ID NO: 3, or the peptide comprising an immunogenic fragment of SEQ ID NO: 2 comprises at least 9 consecutive amino acids of SEQ ID NO: 19.

Advantageously, the peptide comprising an immunogenic fragment of SEQ ID NO: 3 comprises at least 10 consecutive amino acids of SEQ ID NO: 3.

Conveniently, the peptide comprising an immunogenic fragment of SEQ ID NO: 2 comprises at least 10 consecutive amino acids of SEQ ID NO: 19.

Advantageously, the peptide capable of inducing an immune response against a TGFβR2-1a frameshift mutant protein comprises position 121 to 132 of SEQ ID NO: 3, positions 129 to 137 of SEQ ID NO: 3, positions 135 to 146 of SEQ ID NO: 3 or positions 2 to 17 of SEQ ID NO: 19, positions 10 to 18 of SEQ ID NO: 19 or positions 16 to 33 of SEQ ID NO: 19.

Conveniently, the amino acid corresponding to position 121 or 135 of SEQ ID NO: 3 is glycine.

Preferably, the peptide capable of inducing an immune response against a TGFβR2-1a frameshift mutant protein comprises the amino acid sequence of one of SEQ ID NOs: 19 to 25, 124 and 125.

Conveniently, the peptide capable of inducing an immune response against a TGFβR2-1a frameshift mutant protein consists of one of SEQ ID NOs: 19 to 25, 124 and 125.

Advantageously, the immunogenic fragment of SEQ ID NO: 5 comprises at least 10 consecutive amino acids of SEQ ID NO: 26.

Conveniently, the immunogenic fragment of SEQ ID NO: 5 comprises at least 15 consecutive amino acids of SEQ ID NO: 26.

Preferably, the peptide capable of inducing an immune response against a ASTE1-1a frameshift mutant protein comprises the sequence of SEQ ID NO: 26.

Advantageously, the peptide capable of inducing an immune response against a ASTE1-1a frameshift mutant protein consists of SEQ ID NO: 26.

Conveniently, the immunogenic fragment of SEQ ID NO: 7 comprises at least 10 consecutive amino acids of SEQ ID NO: 27.

Advantageously, the immunogenic fragment of SEQ ID NO: 7 comprises at least 13 consecutive amino acids of SEQ ID NO: 27.

Preferably, the peptide capable of inducing an immune response against a TAF1β-1a frameshift mutant protein comprises the sequence of SEQ ID NO: 27.

Conveniently, the peptide capable of inducing an immune response against a TAF1β-1a frameshift mutant protein consists of SEQ ID NO: 27.

Advantageously, the immunogenic fragment of one of SEQ ID NOs: 9-12 comprises at least 10 consecutive amino acids of one of SEQ ID NOs: 9-12.

Conveniently, the immunogenic fragment of one of SEQ ID NOs: 9-12 comprises at least 15 consecutive amino acids of one of SEQ ID NOs: 9-12.

Preferably, the peptide capable of inducing an immune response against a KIAA2018-1a frameshift mutant protein comprises the sequence of SEQ ID NO: 28.

Advantageously, the peptide capable of inducing an immune response against a KIAA2018-1a frameshift mutant protein consists of SEQ ID NO: 28.

Preferably, the immunogenic fragment of one of SEQ ID NOs: 14-18 comprises at least 10 consecutive amino acids of one of SEQ ID NOs: 14-18, respectively, including at least one amino acid from positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18, respectively.

Conveniently, the immunogenic fragment of one of SEQ ID NOs: 14-18 comprises at least 15 consecutive amino acids, of one of SEQ ID NOs: 14-18, respectively, including at least one amino acid from positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18, respectively.

Advantageously, the peptide capable of inducing an immune response against a SLC22A9-1a frameshift mutant protein comprises the sequence of one of SEQ ID NOs: 29-32.

Conveniently, the peptide capable of inducing an immune response against a SLC22A9-1a frameshift mutant protein consists of one of SEQ ID NOs: 29-32.

Preferably, the first peptide is a peptide capable of inducing an immune response to against a TGFβR2-1a frameshift mutant protein, and the second peptide is a peptide capable of inducing an immune response against a ASTE1-1a frameshift mutant protein or a TAF1β-1a frameshift mutant protein.

Advantageously, the peptide mixture comprises at least one further peptide selected from the peptides defined in the first aspect, wherein the at least one further peptide is capable of inducing an immune response against a different frameshift mutant protein from each of the first and second peptides.

Conveniently, the first peptide is a peptide capable of inducing an immune response against a TGFβR2-1a frameshift mutant protein, the second peptide is a peptide capable of inducing an immune response against a ASTE1-1a frameshift mutant protein, and the at least one further peptide is a peptide capable of inducing an immune response against a TAF1β-1a frameshift mutant protein.

Preferably, the peptide mixture further comprises a peptide capable of inducing an immune response against a KIAA2018-1a frameshift mutant protein and/or a peptide capable of inducing an immune response against a SLC22A9-1a frameshift mutant protein.

Advantageously, at least one of the peptides in the peptide mixture comprises a glycine residue at its C-terminus. The glycine residue can serve as a linker for conjugation of the peptide to other molecules. It is therefore envisaged within some embodiments of the present application that the peptide of the invention is linked or conjugated to another molecule (whether by means of such a glycine residue or by other means).

In a second aspect of the invention, there is provided a peptide capable of inducing an immune response against:
a ASTE1-1a frameshift mutant protein, wherein the peptide comprises an immunogenic fragment of SEQ ID NO: 5, wherein the fragment comprises at least 10 consecutive amino acids of SEQ ID NO: 26;
a TAF1β-1a frameshift mutant protein, wherein the peptide comprises an immunogenic fragment of SEQ ID NO: 7, wherein the fragment comprises at least 10 consecutive amino acids of SEQ ID NO: 27;
a KIAA2018-1a frameshift mutant protein, wherein the peptide comprises a immunogenic fragment of one of SEQ ID NOs: 9-12, wherein the fragment comprises at least 8 consecutive amino acids of one of one of SEQ ID NOs: 9-12, including at least one amino acid from positions 13 to 37 of SEQ ID NO: 9, positions 91 to 109 of SEQ ID NO: 10, positions 147 to 167 of SEQ ID NO: 11 and positions 1016 to 1037 of SEQ ID NO: 12; or
a SLC22A9-1a frameshift mutant protein, wherein the peptide comprises an immunogenic fragment of one of SEQ ID NO: 14-18, wherein the fragment comprises at least 8 consecutive amino acids of one of SEQ ID NOs: 14-18, including at least one amino acid from positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18.

Preferably, the peptide capable of inducing an immune response against a TAF1β-1a frameshift mutant protein comprises at least 12 consecutive amino acids from SEQ ID NO: 27.

Advantageously, the peptide capable of inducing an immune response against a TAF1β-1a frameshift mutant protein comprises no more than 27 amino acids.

Preferably, the peptide capable of inducing an immune response comprises no more than eight amino acids from the wild-type amino acid sequence of the corresponding protein.

Conveniently, the peptide comprises a glycine residue at its C-terminus.

In a third aspect of the invention, there is provided a T-cell mixture comprising a first and a second T-cell each independently selected from:
a T-cell specific for a peptide capable of inducing an immune response against TGFβR2-1a frameshift mutant protein as defined in the first aspect above, wherein the T-cell is a non-transfected T-cell;
a T-cell specific for a peptide capable of inducing an immune response against a −1a ASTE1 frameshift mutant protein as defined in the first aspect above;
a T-cell specific for a peptide capable of inducing an immune response against a TAF1β-1a frameshift mutant protein as defined in the first aspect above;
a T-cell specific for a peptide capable of inducing an immune response against a KIAA2018-1a frameshift mutant protein as defined in the first aspect above; and a T-cell specific for a peptide capable of inducing an immune response against a SLC22A9-1a frameshift mutant protein as defined in the first aspect above,
wherein the first T-cell is specific for a peptide which is capable of inducing an immune response against a different frameshift mutant protein from the peptide for which the second T-cell is specific.

In a fourth aspect of the invention, there is provided a T-cell specific for a peptide according to the second aspect above.

In a fifth aspect of the invention, there is provided a T-cell preparation comprising one or more T-cells according to the fourth aspect above.

In a sixth aspect of the invention, there is provided a T-cell receptor, or an antigen-binding fragment thereof, specific for a peptide according to the second aspect above.

In a seventh aspect of the invention, there is provided at least one nucleic acid molecule, wherein the nucleic acid molecule or molecules individually or collectively comprise nucleotide sequences encoding at least two of the peptides defined in the first aspect above or the or each nucleic acid molecule comprises a nucleotide sequence encoding at least one of the peptides according to the second aspect above.

In an eighth aspect of the invention, there is provided a vector comprising the nucleic acid molecule according to the seventh aspect above.

In a ninth aspect of the invention, there is provided a host cell comprising the vector according to the eighth aspect above.

In a tenth aspect of the invention, there is provided a pharmaceutical composition comprising a peptide mixture according to the first aspect above, a peptide according to the second aspect above, a T-cell mixture according to the third aspect above, a T-cell according to the fourth aspect above, a T-cell preparation according to the fifth aspect above, a nucleic acid molecule according to the seventh aspect above, a vector according to the eighth aspect above or a host cell according to the ninth aspect above, and a pharmaceutically-acceptable carrier, diluent or excipient.

In an eleventh aspect of the invention, there is provided a peptide mixture according to the first aspect above, a peptide according to the second aspect above, a T-cell mixture according to the third aspect above, a T-cell according to the fourth aspect above, a T-cell preparation according to the fifth aspect above, a T-cell receptor, or an antigen-binding fragment thereof, according to the sixth aspect above, a nucleic acid molecule according to the seventh aspect above, a vector according to the eighth aspect above, a host cell according to the ninth aspect above, or a pharmaceutical composition according to the tenth aspect above, for use in the treatment and/or prophylaxis of cancer.

Preferably, the cancer is colorectal cancer.

In a twelfth aspect of the invention, there is provided a method of selecting a peptide mixture, peptide, nucleic acid molecule, vector, host cell, T-cell mixture, T-cell, T-cell receptor or a pharmaceutical composition for administration to a patient, comprising:
i) identifying whether a cancer patient has a frameshift mutation in one or more of the TGFβR2 protein, ASTE1 protein, TAF1β protein, KIAA2018 protein and SLC22A9 protein and, if so,
ii) selecting a peptide mixture according to the first aspect above, a peptide according to the second aspect above, a T-cell mixture according to the third aspect above, a T-cell according to the fourth aspect above, a T-cell preparation according to the fifth aspect above, a T-cell receptor, or an antigen-binding fragment thereof, according to the sixth aspect above, a nucleic acid molecule according to the seventh aspect above, a vector according to the eighth aspect above, a host cell according to the ninth aspect above or a pharmaceutical composition according to the tenth aspect above.

In a thirteenth aspect of the invention, there is provided a method of treating cancer comprising administering, to a patient in need thereof, a peptide mixture according to the first aspect above, a peptide according to the second aspect above, a T-cell mixture according to the third aspect above, a T-cell according to the fourth aspect above, a T-cell preparation according to the fifth aspect above, a T-cell receptor, or an antigen-binding fragment thereof, according to the sixth aspect above, a nucleic acid molecule according to the seventh aspect above, a vector according to the eighth aspect above, a host cell according to the ninth aspect above or a pharmaceutical composition according to the tenth aspect above.

The term “peptide”, as used herein, refers to a polymer of amino acid residues that is (or has a sequence that corresponds to) a fragment of a longer protein. The term also applies to amino acid polymers in which one or more amino acid residues is a modified residue, or a non-naturally occurring residue, such as an artificial chemical mimetic of a corresponding naturally occurring amino acid, as well as to naturally occurring amino acid polymers. The peptide may be linked to another agent or moiety.

The term “fragment”, as used herein, refers to a series of consecutive amino acids from a longer polypeptide or protein.

The percentage “identity” between two sequences may be determined using the BLASTP algorithm version 2.2.2 Altschul, Stephen F., Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402), using default parameters. In particular, the BLAST algorithm can be accessed in the internet using the URL https://blast.ncbi.nlm.nih.gov/Blast.cgi.

The term “immune response”, as used herein, refers in some embodiments to a T-cell mediated immune response (i.e. T-cell activation) upon recognition of a peptide. The T-cell response may be a HLA-I mediated T-cell response and/or a HLA-II mediated T-cell response. The immune response may be a response by any alpha beta (αβ) T-cells and/or gamma delta (γδ) T-cells, such that the peptides may or may not be presented to the T-cells by major histocompatibility (MHC) molecules on the surface of antigen-presenting cells.

The term “frameshift mutant”, as used herein, refers to a polypeptide encoded by a nucleic acid sequence having an addition or deletion of one or two nucleotides compared to the wild-type sequence of the nucleic acid, thereby resulting in different codons as of the point of mutation.

The term “−1a frameshift mutant”, as used herein, refers to a polypeptide resulting from the deletion of a single nucleotide from the wild-type nucleic acid sequence.

The term “−1a frameshift mutation” refers to a change in the amino acid sequence of a polypeptide compared to the wild-type amino acid sequence of the polypeptide, resulting from the deletion of a single nucleotide from the nucleic acid sequence encoding that polypeptide.

The term “mutTGFβR2”, as used herein, refers to a TGFβR2 protein which has a −1a frameshift mutation. The amino acid sequence of mutTGFβR2 is shown in SEQ ID NO: 2.

The term “mutASTE1”, as used herein, refers to a ASTE1 protein which has a −1a frameshift mutation. The amino acid sequence of mutASTE1 is shown in SEQ ID NO: 5.

The term “mutTAF1B”, as used herein, refers to a TAF1B protein which has a −1a frameshift mutation. The amino acid sequence of mutTAF1B is shown in SEQ ID NO: 7.

The term “mutKIAA2018”, as used herein, refers to a KIAA2018 protein which has a −1a frameshift mutation.

The term “mutKIAA2018(pos13)”, as used herein, refers to a KIAA2018 protein which has a −1a frameshift mutation at position 13 of the amino acid sequence thereof. In other words, the mutKIAA2018(pos13) protein has a mutated amino acid sequence, compared to the wild-type protein, starting at position 13 of the amino acid sequence thereof. The amino acid sequence of mutKIAA2018(pos13) is shown in SEQ ID NO: 9.

The term “mutKIAA2018(pos91)”, as used herein, refers to a KIAA2018 protein which has a −1a frameshift mutation at position 91 of the amino acid sequence thereof. In other words, the mutKIAA2018(pos91) protein has a mutated amino acid sequence, compared to the wild-type protein, starting at position 91 of the amino acid sequence thereof. The amino acid sequence of mutKIaa2018(pos91) is shown in SEQ ID NO: 10.

The term “mutKIAA2018(pos147)”, as used herein, refers to a KIAA2018 protein which has a frameshift mutation at position 147 of the amino acid sequence thereof. In other words, the mutKIAA2018(pos147) protein has a mutated amino acid sequence, compared to the wild-type protein, starting at position 147 of the amino acid sequence thereof. The amino acid sequence of mutKIAA2018(pos147) is shown in SEQ ID NO: 11.

The term “mutKIAA2018(pos1016)”, as used herein, refers a KIAA2018 protein which has a frameshift mutation at position 1016 of the amino acid sequence thereof. In other words, the mutKIAA2018(pos1016) protein has a mutated amino acid sequence, compared to the wild-type protein, starting at position 1016 of the amino acid sequence thereof. The amino acid sequence of mutKIAA2018(pos1016) is shown in SEQ ID NO: 12.

The term “mutSLC22A9”, as used herein, refers to a SLC22A9 protein which has a −1a frameshift mutation.

The term “mutSLC22A9(pos327)”, as used herein, refers to a SLC22A9 protein which has a frameshift mutation at position 327 of the amino acid sequence thereof. In other words, the mutSLCC22A9(pos327) protein has a mutated amino acid sequence, compared to the wild-type sequence, starting at position 327 thereof. The amino acid sequence of mutSLCC22A(pos327) is shown in SEQ ID NO: 14.

The term “mutSLC22A9(pos335)”, as used herein, refers to a SLC22A9 protein which has a frameshift mutation at position 335 of the amino acid sequence thereof. In other words, the mutSLCC22A9(pos335) protein has a mutated amino acid sequence, compared to the wild-type sequence, starting at position 335 thereof. The amino acid sequence of mutSLCC22A9(pos335) is shown in SEQ ID NO: 15.

The term “mutSLC22A9(pos533)”, as used herein, refers to a SLC22A9 protein which has a frameshift mutation at position 533 of the amino acid sequence thereof. In other words, the mutSLCC22A9(pos533) protein has a mutated amino acid sequence, compared to the wild-type sequence, starting at position 533 thereof. The amino acid sequence of mutSLCC22A9(pos533) is shown in SEQ ID NO: 16.

The term “amino acid substitution”, as used herein, refers to the replacement of an amino acid in a polypeptide with a different amino acid, compared to the wild-type amino acid sequence of the polypeptide.

The term “peptide mixture”, as used herein, refers to two or more peptides which are mixed but not chemically combined. The mixtures may be present as a dry powder, solution, suspension or colloid, and may be homogeneous or heterogeneous.

The term “nucleic acid” or “nucleic acid molecule”, as used herein, refers to a polymer of multiple nucleotides. The nucleic acid may comprise naturally occurring nucleotides or may comprise artificial nucleotides such as peptide nucleotides, morpholin and locked nucleotides as well as glycol nucleotides and threose nucleotides.

The term “nucleotide”, as used herein, refers to naturally occurring nucleotides and synthetic nucleotide analogues that are recognised by cellular enzymes.

The term “pharmaceutical composition”, as used herein, means a pharmaceutical preparation suitable for administration to an intended human or animal subject for therapeutic purposes.

### BRIEF DESCRIPTION OF THE FIGURES

FIG. 1 shows the development of the TGFβR2 consensus sequence and peptides.

FIG. 2 is a UPLC trace of freshly prepared crude fsp5 (SEQ ID NO: 19).

FIG. 3 is a UPLC trace of purified fsp5 (SEQ ID NO: 19) in solution before lyophilisation.

FIG. 4 is a UPLC trace of purified fsp5 (SEQ ID NO: 19) after lyophilisation.

FIG. 5 is a HPLC trace of crude fsp5 (SEQ ID NO: 19) after storage for three days at room temperature, followed by reconstitution and lyophilisation.

FIG. 6 is a UPLC trace of purified fsp1 (SEQ ID NO: 20).

FIG. 7 is a UPLC trace of purified fsp2 (SEQ ID NO: 21).

FIG. 8 is a UPLC trace of purified fsp3 (SEQ ID NO: 22).

FIG. 9 is a UPLC trace of purified fsp4 (SEQ ID NO: 23).

FIG. 10 is a graph showing T-cell proliferation after one round of stimulation with a peptide mixture containing fsp2 (SEQ ID NO: 21) and fsp4 (SEQ ID NO: 23).

FIG. 11 is a graph showing T-cell proliferation after a second round of stimulation with a peptide mixture containing fsp2 (SEQ ID NO: 21) and fsp4 (SEQ ID NO: 23).

FIG. 12 is a graph showing an alternative presentation of the T-cell proliferation in samples from Donors 2, 3, and 4 shown in FIG. 3.

FIG. 13 is a graph showing T-cell proliferation induced by fsp2 (SEQ ID NO: 21), fsp6 (SEQ ID NO: 24) and fsp7 (SEQ ID NO: 123), after stimulation with a peptide mixture containing fsp2 (SEQ ID NO: 21), fsp8 (SEQ ID NO: 26) and fsp9 (SEQ ID NO: 27).

FIG. 14 is a graph showing T-cell proliferation after two rounds of in vitro stimulation of PMBCs from healthy donors with a peptide mixture containing fsp2 (SEQ ID NO: 21), fsp8 (SEQ ID NO: 26) and fsp9 (SEQ ID NO: 27).

FIG. 15 is a graph showing T-cell proliferation after two and three rounds of in vitro stimulation of PMBCs from a healthy donor with a peptide mixture containing fsp2 (SEQ ID NO: 21), fsp8 (SEQ ID NO: 26) and fsp9 (SEQ ID NO: 27).

FIG. 16 is a graph showing T-cell proliferation induced by fsp2 (SEQ ID NO: 21), fsp8 (SEQ ID NO: 26) or fsp9 (SEQ ID NO: 27), individually, or a peptide mixture containing fsp2 (SEQ ID NO: 21), fsp8 (SEQ ID NO: 26) and fsp9 (SEQ ID NO: 27), after in vitro stimulation of PMBCs from a healthy donor with a peptide mixture containing fsp2 (SEQ ID NO: 21), fsp8 (SEQ ID NO: 26) and fsp9 (SEQ ID NO: 27).

FIG. 17 is a graph showing T-cell proliferation induced by fsp2 (SEQ ID NO: 21), fsp8 (SEQ ID NO: 26) or fsp9 (SEQ ID NO: 27), individually, or a peptide mixture containing fsp2 (SEQ ID NO: 21), fsp8 (SEQ ID NO: 26) and fsp9 (SEQ ID NO: 27), after in vitro stimulation of PMBCs from a healthy donor with a peptide mixture containing fsp2 (SEQ ID NO: 21), fsp8 (SEQ ID NO: 26) and fsp9 (SEQ ID NO: 27).

FIG. 18 is a graph showing T-cell proliferation induced by fsp2 (SEQ ID NO: 21), fsp8 (SEQ ID NO: 26) or fsp9 (SEQ ID NO: 27), individually, or a peptide mixture containing fsp2 (SEQ ID NO: 21), fsp8 (SEQ ID NO: 26) and fsp9 (SEQ ID NO: 27), after in vitro stimulation of PMBCs from a healthy donor with a peptide mixture containing fsp2 (SEQ ID NO: 21), fsp8 (SEQ ID NO: 26) and fsp9 (SEQ ID NO: 27).

FIG. 19 is a graph showing the peptide-specific T-cell proliferation induced by fsp11 (SEQ ID NO: 29) or fsp13 (SEQ ID NO: 31), individually, or a peptide mixture containing fsp11 (SEQ ID NO: 29) and fsp13 (SEQ ID NO: 31), after in vitro stimulation of PBMCs from a healthy donor with a peptide mixture containing fsp10 (SEQ ID NO: 28), fsp11 (SEQ ID NO: 29) and fsp13 (SEQ ID NO: 31).

FIG. 20 is a graph showing the T-cell proliferation induced by a peptide mixture containing fsp10 (SEQ ID NO: 28), fsp11 (SEQ ID NO: 29) and fsp13 (SEQ ID NO: 31) at a high concentration (10 μM) of each peptide or at a low concentration (3.3 μM) of each peptide, after two rounds of in vitro stimulation of PMBCs from a healthy donor with a peptide mixture containing fsp10 (SEQ ID NO: 28), fsp11 (SEQ ID NO: 29) and fsp13 (SEQ ID NO: 31).

FIG. 21 is a graph showing the T-cell proliferation induced by fsp15 (SEQ ID NO: 127) after in vitro stimulation of PMBCs from a healthy donor with a peptide mixture containing fsp17 (SEQ ID NO: 126), fsp15 (SEQ ID NO: 127) and fsp16 (SEQ ID NO: 128).

FIG. 22 is a graph showing the T-cell proliferation induced by fsp17 (SEQ ID NO: 126), fsp15 (SEQ ID NO: 127) or fsp16 (SEQ ID NO: 128), individually, or by a mixture containing fsp17 (SEQ ID NO: 126), fsp15 (SEQ ID NO: 127) and fsp16 (SEQ ID NO: 128), after two rounds of in vitro stimulation of PMBCs from a healthy donor with a peptide mixture containing fsp17 (SEQ ID NO: 126), fsp15 (SEQ ID NO: 127) and fsp16 (SEQ ID NO: 128).

### BRIEF DESCRIPTION OF THE SEQUENCE LISTING

SEQ ID NO: 1 is the full length wild-type TGFβR2 protein.

SEQ ID NO: 2 is the full length −1a frameshifted TGFβR2 protein.

SEQ ID NO: 3 is the full length −1a frameshifted TGFβR2 protein, having amino acid substitutions at position 121 and 135. Free text in sequence listing: Modified peptide.

SEQ ID NO: 4 is the full length wild-type ASTE1 protein.

SEQ ID NO: 5 is the full length −1a frameshifted ASTE1 protein.

SEQ ID NO: 6 is the full length wild-type TAF1β protein.

SEQ ID NO: 7 is the full length −1a frameshifted TAF1β protein.

SEQ ID NO: 8 is the full length wild-type KIAA2018 protein.

SEQ ID NO: 9 is the full length −1a frameshifted KIAA2018 protein, having a frameshift mutation at position 13.

SEQ ID NO: 10 is the full length −1a frameshifted KIAA2018 protein, having a frameshift mutation at position 91.

SEQ ID NO: 11 is the full length −1a frameshifted KIAA2018 protein, having a frameshift mutation at position 147.

SEQ ID NO: 12 is the full length −1a frameshifted KIAA2018 protein, having a frameshift mutation at position 1016.

SEQ ID NO: 13 is the full length wild-type SLC22A9 protein.

SEQ ID NO: 14 is the full length −1a frameshifted SLC22A9 protein, having a frameshift mutation at position 327.

SEQ ID NO: 15 is the full length −1a frameshifted SLC22A9 protein, having a frameshift mutation at position 335.

SEQ ID NO: 16 is the full length −1a frameshifted SLC22A9 protein, having a frameshift mutation at position 533.

SEQ ID NO: 17 is the full length −1a frameshifted SLC22A9 protein, having a frameshift mutation at position 327 and an amino acid substitution at position 354. Free text in sequence listing: Modified peptide.

SEQ ID NO: 18 is the full length −1a frameshifted SLC22A9 protein, having a frameshift mutation at position 335 and an amino acid substitution at position 344. Free text in sequence listing: Modified peptide.

SEQ ID NO: 19 is a 33-amino acid peptide of SEQ ID NO: 2, referred to herein as fsp5.

SEQ ID NO: 20 is a peptide −1a frameshifted TGFβR2, referred to herein as fsp1.

SEQ ID NO: 21 is a peptide of −1a frameshifted TGFβR2, referred to herein as fsp2. Free text in sequence listing: Modified peptide.

SEQ ID NO: 22 is a peptide of −1a frameshifted TGFβR2, referred to herein as fsp3.

SEQ ID NO: 23 is a peptide of −1a frameshifted TGFβR2, referred to herein as fsp4. Free text in sequence listing: Modified peptide.

SEQ ID NO: 24 is a peptide of −1a frameshifted TGFβR2, referred to herein as fsp6.

SEQ ID NO: 25 is a peptide of −1a frameshifted TGFβR2, referred to herein as fsp6a. Free text in sequence listing: Modified peptide.

SEQ ID NO: 26 is a peptide of −1a frameshifted ASTE1, referred to herein as fsp8.

SEQ ID NO: 27 is a peptide of −1a frameshifted TAF1βR, referred to herein as fsp9.

SEQ ID NO: 28 is a peptide of −1a frameshifted KIAA2018, referred to herein as fsp10.

SEQ ID NO: 29 is a peptide of −1a frameshifted SLC22A9, referred to herein as fsp11.

SEQ ID NO: 30 is a peptide of −1a frameshifted SLC22A9, referred to herein as fsp12. Free text in sequence listing: Modified peptide.

SEQ ID NO: 31 is a peptide of −1a frameshifted SLC22A9, referred to herein as fsp13.

SEQ ID NO: 32 is a peptide of −1a frameshifted SLC22A9, referred to herein as fsp14. Free text in sequence listing: Modified peptide.

SEQ ID NO: 33 is a prior art peptide of mutTGFβR2.

SEQ ID NO: 34 is a prior art peptide of mutTGFβR2.

SEQ ID NO: 35 is a prior art peptide of mutTGFβR2.

SEQ ID NO: 36 is a prior art peptide of mutTGFβR2.

SEQ ID NO: 37 is a prior art peptide of mutTGFβR2.

SEQ ID NO: 38 is a prior art peptide of mutTGFβR2.

SEQ ID NO: 39 is a prior art peptide of mutTGFβR2.

SEQ ID NO: 40 is a manually-predicted consensus sequence of mutTGFβR2.

SEQ ID NO: 41 is an epitope of mutTGFβR2 predicted by SYFPEITHI.

SEQ ID NO: 42 is an epitope of mutTGFβR2 predicted by SYFPEITHI.

SEQ ID NO: 43 is an epitope of mutTGFβR2 predicted by SYFPEITHI.

SEQ ID NO: 44 is an epitope of mutTGFβR2 predicted by SYFPEITHI.

SEQ ID NO: 45 is an epitope of mutTGFβR2 predicted by SYFPEITHI.

SEQ ID NO: 46 is an epitope of mutTGFβR2 predicted by SYFPEITHI.

SEQ ID NO: 47 is an epitope of mutTGFβR2 predicted by SYFPEITHI.

SEQ ID NO: 48 is an epitope of mutTGFβR2 predicted by SYFPEITHI.

SEQ ID NO: 49 is an epitope of mutTGFβR2 predicted by SYFPEITHI.

SEQ ID NO: 50 is a sequence of mutASTE1 used for searching for predicted epitopes.

SEQ ID NO: 51 is an epitope of mutASTE1 predicted by SYFPEITHI.

SEQ ID NO: 52 is an epitope of mutASTE1 predicted by SYFPEITHI.

SEQ ID NO: 53 is an epitope of mutASTE1 predicted by SYFPEITHI.

SEQ ID NO: 54 is an epitope of mutASTE1 predicted by SYFPEITHI.

SEQ ID NO: 55 is a predicted nested epitope of mutASTE1.

SEQ ID NO: 56 is an epitope of mutASTE1 predicted by SYFPEITHI.

SEQ ID NO: 57 is an epitope of mutASTE1 predicted by SYFPEITHI.

SEQ ID NO: 58 is an epitope of mutASTE1 predicted by SYFPEITHI.

SEQ ID NO: 59 is a sequence of mutTAF1β used for searching for predicted epitopes.

SEQ ID NO: 60 is an epitope of mutTAF1β predicted by SYFPEITHI.

SEQ ID NO: 61 is an epitope of mutTAF1β predicted by SYFPEITHI.

SEQ ID NO: 62 is an epitope of mutTAF1β predicted by SYFPEITHI.

SEQ ID NO: 63 is an epitope of mutTAF1β predicted by SYFPEITHI.

SEQ ID NO: 64 is an epitope of mutTAF1β predicted by SYFPEITHI.

SEQ ID NO: 65 is an epitope of mutTAF1β predicted by SYFPEITHI.

SEQ ID NO: 66 is an epitope of mutTAF1β predicted by SYFPEITHI.

SEQ ID NO: 67 is an epitope of mutTAF1β predicted by SYFPEITHI.

SEQ ID NO: 68 is an epitope of mutTAF1β predicted by SYFPEITHI.

SEQ ID NO: 69 is a predicted nested epitope of mutTAF1β.

SEQ ID NO: 70 is an epitope of mutTAF1β predicted by SYFPEITHI.

SEQ ID NO: 71 is an epitope of mutTAF1β predicted by SYFPEITHI.

SEQ ID NO: 72 is an epitope of mutTAF1β predicted by SYFPEITHI.

SEQ ID NO: 73 is an epitope of mutTAF1β predicted by SYFPEITHI.

SEQ ID NO: 74 is an epitope of mutTAF1β predicted by SYFPEITHI.

SEQ ID NO: 75 is an epitope of mutTAF1β predicted by SYFPEITHI.

SEQ ID NO: 76 is a sequence of mutKIAA2018 used for searching for predicted epitopes.

SEQ ID NO: 77 is an epitope of mutKIAA2018 predicted by SYFPEITHI.

SEQ ID NO: 78 is an epitope of mutKIAA2018 predicted by SYFPEITHI.

SEQ ID NO: 79 is an epitope of mutKIAA2018 predicted by SYFPEITHI.

SEQ ID NO: 80 is an epitope of mutKIAA2018 predicted by SYFPEITHI.

SEQ ID NO: 81 is an epitope of mutKIAA2018 predicted by SYFPEITHI.

SEQ ID NO: 82 is a predicted nested epitope of mutKIAA2018.

SEQ ID NO: 83 is an epitope of mutKIAA2018 predicted by SYFPEITHI.

SEQ ID NO: 84 is an epitope of mutKIAA2018 predicted by SYFPEITHI.

SEQ ID NO: 85 is an epitope of mutKIAA2018 predicted by SYFPEITHI.

SEQ ID NO: 86 is an epitope of mutKIAA2018 predicted by SYFPEITHI.

SEQ ID NO: 87 is an epitope of mutKIAA2018 predicted by SYFPEITHI.

SEQ ID NO: 88 is an epitope of mutKIAA2018 predicted by SYFPEITHI.

SEQ ID NO: 89 is an epitope of mutKIAA2018 predicted by SYFPEITHI.

SEQ ID NO: 90 is an epitope of mutKIAA2018 predicted by SYFPEITHI.

SEQ ID NO: 91 is an epitope of mutKIAA2018 predicted by SYFPEITHI.

SEQ ID NO: 92 is an epitope of mutKIAA2018 predicted by SYFPEITHI.

SEQ ID NO: 93 is an epitope of mutKIAA2018 predicted by SYFPEITHI.

SEQ ID NO: 94 is an epitope of mutKIAA2018 predicted by SYFPEITHI.

SEQ ID NO: 95 is an epitope of mutKIAA2018 predicted by SYFPEITHI.

SEQ ID NO: 96 is a sequence of mutSLC22A9 used for searching for predicted epitopes.

SEQ ID NO: 97 is an epitope of mutSLC22A9 predicted by SYFPEITHI.

SEQ ID NO: 98 is an epitope of mutSLC22A9 predicted by SYFPEITHI.

SEQ ID NO: 99 is an epitope of mutSLC22A9 predicted by SYFPEITHI.

SEQ ID NO: 100 is an epitope of mutSLC22A9 predicted by SYFPEITHI.

SEQ ID NO: 101 is an epitope of mutSLC22A9 predicted by SYFPEITHI.

SEQ ID NO: 102 is an epitope of mutSLC22A9 predicted by SYFPEITHI.

SEQ ID NO: 103 is an epitope of mutSLC22A9 predicted by SYFPEITHI.

SEQ ID NO: 104 is an epitope of mutSLC22A9 predicted by SYFPEITHI.

SEQ ID NO: 105 is an epitope of mutSLC22A9 predicted by SYFPEITHI.

SEQ ID NO: 106 is an epitope of mutSLC22A9 predicted by SYFPEITHI.

SEQ ID NO: 107 is an epitope of mutSLC22A9 predicted by SYFPEITHI.

SEQ ID NO: 108 is an epitope of mutSLC22A9 predicted by SYFPEITHI.

SEQ ID NO: 109 is an epitope of mutSLC22A9 predicted by SYFPEITHI.

SEQ ID NO: 110 is an epitope of mutSLC22A9 predicted by SYFPEITHI.

SEQ ID NO: 111 is a predicted nested epitope of mutSLC22A9.

SEQ ID NO: 112 is a predicted nested epitope of mutSLC22A9.

SEQ ID NO: 113 is an epitope of mutSLC22A9 predicted by SYFPEITHI.

SEQ ID NO: 114 is an epitope of mutSLC22A9 predicted by SYFPEITHI.

SEQ ID NO: 115 is an epitope of mutSLC22A9 predicted by SYFPEITHI.

SEQ ID NO: 116 is an epitope of mutSLC22A9 predicted by SYFPEITHI.

SEQ ID NO: 117 is an epitope of mutSLC22A9 predicted by SYFPEITHI.

SEQ ID NO: 118 is an epitope of mutSLC22A9 predicted by SYFPEITHI.

SEQ ID NO: 119 is an epitope of mutSLC22A9 predicted by SYFPEITHI.

SEQ ID NO: 120 is an epitope of mutSLC22A9 predicted by SYFPEITHI.

SEQ ID NO: 121 is an epitope of mutSLC22A9 predicted by SYFPEITHI.

SEQ ID NO: 122 is an epitope of mutSLC22A9 predicted by SYFPEITHI.

SEQ ID NO: 123 is a peptide of −1a frameshifted TGFβR2, referred to herein as fsp7.

SEQ ID NO: 124 is a peptide of −1a frameshifted TGFβR2, referred to herein as fsp1a.

SEQ ID NO: 125 is a peptide of −1a frameshifted TGFβR2, referred to herein as fsp1b. Free text in sequence listing: Modified peptide.

SEQ ID NO: 126 is a peptide of −1a frameshifted TGFβR2, herein referred to as fsp17. Free text in sequence listing: modified peptide.

SEQ ID NO: 127 is a peptide of −1a frameshifted ASTE1, herein referred to as fsp15.

SEQ ID NO: 128 is a peptide of −1a frameshifted TAF1β, herein referred to as fsp16.

SEQ ID NO: 129 is a peptide of −1a frameshifted TGFβR2, herein referred to as fsp17a.

### DETAILED DESCRIPTION

The invention relates, in general terms, to a peptide mixture comprising two or more peptides derived from TGFβR2, ASTE1, TAF1β, KIAA2018 and SLC22A9, each having a frameshift mutation, and to peptides derived from ASTE1, TAF1β, KIAA2018 and SLC22A9, each having a frameshift mutation. Each peptide comprises a fragment of the relevant frameshift mutant protein and is able to induce an immune response against the relevant frameshift mutant protein. The peptide mixture comprises at least two peptides which are able to induce an immune response against different frameshift mutant proteins. In some embodiments, each frameshift mutant protein is a −1a frameshift mutant (referred to herein as “mutTGFβR2”, “mutASTE1”, “mutTAF1β”, “mutKIAA2018” and “mutSLC22A9”, respectively). The amino acid sequences of each of the TGFβR2, ASTE1, TAF1@, KIAA2018 and SLC22A9-1a frameshift mutant proteins is shown in SEQ ID NOs: 2, 5, 7, 9-12 and 14-16.

Each of the peptides comprises a sequence which corresponds to an immunogenic fragment of the −1a frameshifted mutant protein from which the peptide is derived (i.e. one of mutTGFβR2 (SEQ ID NO: 2), ASTE1 (SEQ ID NO: 5), TAF1β (SEQ ID NO: 7), KIAA2018 (SEQ ID NOs: 9-12) and SLC22A9 (SEQ ID NOs: 14-16)) displayed by HLA-I or HLA-II molecules on the surface of cells, and/or to which individuals generally have a reactive T-cell in their T-cell repertoire. Each peptide, whether present as a single peptide preparation or in a peptide mixture, is able to induce an immune response against the −1a frameshifted mutant protein from which the peptide in question is derived. Preferably, the immune response is a T-cell response, comprising both HLA-1-restricted T-cells, such as CD8+ T-cells, and HLA-II-restricted T-cells, such as CD4+ T-cells. In particular, the peptides of the invention, and the peptides in the peptide mixtures of the invention, may encompass multiple nested epitopes, such that each peptide may comprise epitopes for more than one HLA allele. This provides the advantage that the peptides are capable of inducing an immune response in patients having different HLA alleles, such that the peptides are useful as a universal treatment and/or vaccine. In addition, the peptides of the invention contain few or no amino acids from the wild-type amino acid sequence of the protein from which they are derived. In other words, the peptides of the invention are mostly or entirely derived from the amino acid sequence of the corresponding protein which has been altered as a result of the frameshift mutation. This provides the advantage that the risk of the peptides inducing an autoimmune response is minimised. Furthermore, the peptides of the invention can contain a glycine residue at the C-terminus thereof, which serves as a linker for conjugation of the peptide to other molecules, such as other peptides, carriers, adjuvants, etc.

Features which apply generally to any and all of the peptides of the present invention, and the peptides of the peptide mixtures of the present invention, are set out immediately below, while features applying more specifically to a peptide of a particular frameshift mutant protein are described later.

Generally Applicable Features

In some embodiments, the immunogenic fragment of the or each peptide independently comprises at least 8, at least 9, at least 10, at least 12, at least 14, at least 16, at least 18, at least 20, at least 22, at least 24, at least 26, at least 28, at least 30 or at least 32 amino acids.

In some embodiments, the immunogenic fragment of the or each peptide independently comprises no more than 100, 50 or 40 amino acids. For example, the immunogenic fragment may comprise no more than 35, 33, 31, 29, 27, 25, 23, 21, 19, 17, 15, 13, 11 or 9 amino acids.

In some embodiments, the or each peptide independently comprises at least 9, at least 10, at least 12, at least 14, at least 16, at least 18, at least 20, at least 22, at least 24, at least 26, at least 28, at least 30 or at least 32 amino acids.

In some embodiments, the or each peptide comprises no more than 100, 50 or 40 amino acids. For example, the peptide may comprise no more than 35, 33, 31, 29, 27, 25, 23, 21, 19, 17, 15, 13, 11 or 9 amino acids.

Thus, in some embodiments, the or each peptide comprises other amino acids outside of the immunogenic fragment of the relevant frameshifted protein. However, in other embodiments, the or each peptide is the same length as the immunogenic fragment, such that the peptide is an immunogenic fragment of the relevant frameshift mutant protein.

In some embodiments, the or each peptide comprises no more than 8 amino acids from the wild-type amino acid sequence of the corresponding protein. In some embodiments, the or each peptide comprises no more than 7, no more than 6, no more than 5, no more than 4, no more than 3, no more than 2 or no more than 1 amino acid from the wild-type amino acid sequence of the corresponding protein.

When a peptide comprises one or more amino acids from the wild-type amino acid sequence of the corresponding protein, these amino acids are preferably at the N-terminus of the protein.

In some embodiments, the or each peptide comprises no amino acids from the wild-type amino acid sequence of the corresponding protein. Thus, in these embodiments, the or each peptide consists only of amino acids from the amino acid sequence resulting from the frameshift mutation in the corresponding protein.

In some embodiments, the or each peptide has a glycine residue at the C-terminus thereof (i.e. the C-terminus is a glycine residue).

Sequence Identity to the Frameshifted Protein Outside of the Immunogenic Fragment

The or each peptide may have at least 70% sequence identity to the relevant frameshift mutant protein outside of the immunogenic fragment. In some embodiments, the or each peptide independently has at least 75%, at least 80%, at least 85%, at least 90%, at least 92%, at least 93%, at least 94% or at least 95% sequence identity to the relevant frameshift mutant protein outside of the immunogenic fragment. In some embodiments, the or each peptide independently has 100% sequence identity to the relevant frameshift mutant protein outside of the immunogenic fragment.

Position of the Fragment within the Peptide

In some embodiments, the immunogenic fragment starts at position one, two, three, four, five, six, seven, eight, nine, ten or eleven from the N-terminus of the peptide. In some embodiments, the immunogenic fragment ends at position one, two three, four, five, six, seven, eight, nine, ten or eleven from the C-terminus of the peptide. In other embodiments, the immunogenic fragment is the C-terminus or the N-terminus of the peptide.

ASTE1 Peptide

In some embodiments, in the peptide capable of inducing an immune response against a ASTE1-1a frameshift mutant protein (mutASTE1; SEQ ID NO: 5), the immunogenic fragment comprises at least 8 consecutive amino acids of SEQ ID NO: 26. In some embodiments, the immunogenic fragment comprises at least 9 consecutive amino acids of SEQ ID NO: 26. In some embodiments, the immunogenic fragment comprises at least 10 consecutive amino acids of SEQ ID NO: 26. In some embodiments, the immunogenic fragment comprises at least 12 consecutive amino acids of SEQ ID NO: 26. In some embodiments, the immunogenic fragment comprises at least 15 consecutive amino acids of SEQ ID NO: 26. In some embodiments, the immunogenic fragment comprises at least 20 consecutive amino acids of SEQ ID NO: 26. In other embodiments, the immunogenic fragment comprises at least 25 consecutive amino acids of SEQ ID NO: 26.

In some embodiments, the immunogenic fragment comprises no more than 25 consecutive amino acids of SEQ ID NO: 26. In some embodiments, the peptide comprises no more than 31 consecutive amino acids of SEQ ID NO: 127.

In some embodiments, the peptide comprises at least 10 amino acids. In some embodiments, the peptide comprises at least 12 amino acids. In some embodiments, the peptide comprises at least 15 amino acids. In some embodiments, the peptide comprises 20 amino acids. In other embodiments, the peptide comprises at least 25 amino acids.

In some embodiments, the peptide comprises no more than 40 amino acids. In some embodiments, the peptide comprises no more than 31 amino acids. In other embodiments, the peptide comprises no more than 25 amino acids.

In some embodiments, the immunogenic fragment of mutASTE1 (SEQ ID NO: 5) comprises no more than 8 amino acids from the wild-type amino acid sequence of ASTE1 (SEQ ID NO: 4). In some embodiments, the immunogenic fragment of mutASTE1 (SEQ ID NO: 5) comprises no more than 5 amino acids from the wild-type amino acid sequence of ASTE1 (SEQ ID NO: 4). In some embodiments, the immunogenic fragment of mutASTE1 (SEQ ID NO: 5) comprises no more than 3 amino acids from the wild-type amino acid sequence of ASTE1 (SEQ ID NO: 4). In some embodiments, the immunogenic fragment of mutASTE1 (SEQ ID NO: 5) does not contain any amino acids from the wild-type amino acid sequence of ASTE1 (SEQ ID NO: 4). In some embodiments, the immunogenic fragment has only three amino acids from the wild-type amino acid sequence of ASTE1 (SEQ ID NO: 4).

In some embodiments, the peptide capable of inducing an immune response against mutASTE1 (SEQ ID NO: 5) comprises no more than 8 amino acids from the wild-type amino acid sequence of ASTE1 (SEQ ID NO: 4). In some embodiments, the peptide does not contain more than 5 amino acids from the wild-type sequence of ASTE1 (SEQ ID NO: 4). In some embodiments, the peptide does not contain more than 3 amino acids from the wild-type sequence of ASTE1 (SEQ ID NO: 4). In some embodiments, the peptide contains only 3 amino acids from the wild-type amino acid sequence of ASTE1 (SEQ ID NO: 4). In some embodiments, the peptide does not contain any amino acids from the wild-type amino acid sequence of ASTE1 (SEQ ID NO: 4).

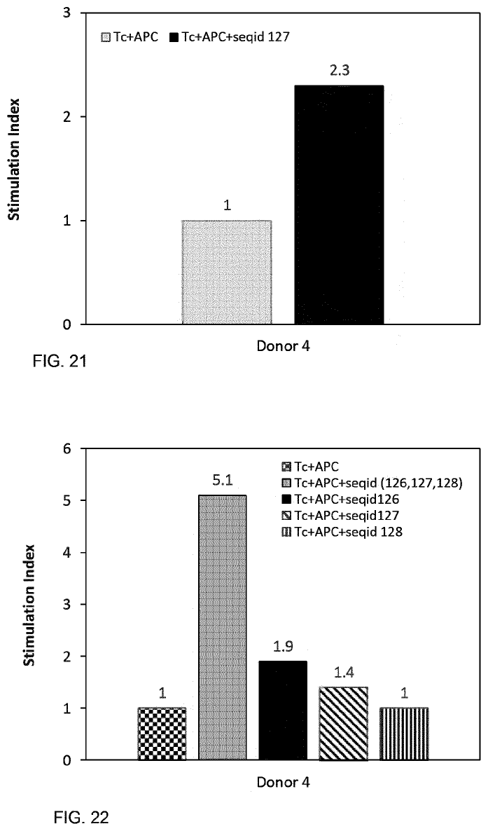

In some embodiments, the immunogenic fragment of mutASTE1 (SEQ ID NO: 5) or the peptide capable of inducing an immune response against mutASTE1 (SEQ ID NO: 5) comprises positions 631 and 632 of SEQ ID NO: 5, wherein the amino acid at position 631 of SEQ ID NO: 5 corresponds to position 631 of wild-type ASTE1 (SEQ ID NO: 4) and the amino acid at position 632 of SEQ ID NO: 5 is the first amino acid of the amino acid sequence resulting from the frameshift mutation in mutASTE1 (SEQ ID NO: 5). In some embodiments, the immunogenic fragment or peptide comprises position 630 to position 632, position 629 to position 632, position 628 to position 632, or position 627 to position 632 of SEQ ID NO: 5. In some embodiments, the immunogenic fragment or peptide has only 3 amino acids from the wild-type amino acid sequence of ASTE1 (SEQ ID NO: 4), which are positions 629, 630 and 631 of SEQ ID NO: 4 (i.e. positions 629, 630 and 631 of SEQ ID NO: 5). In particular, FIGS. 21 and 22 show that a peptide (fsp15; SEQ ID NO: 127) having only 3 amino acids from the wild-type amino acid sequence of ASTE1 (SEQ ID NO: 4) is capable of inducing a T-cell response, and therefore is immunogenic, when administered alone or in a peptide mixture.

When an immunogenic fragment or a peptide comprises one or more amino acids from the wild-type amino acid sequence of ASTE1 (SEQ ID NO: 4), these amino acids are preferably at the N-terminus of the immunogenic fragment or peptide. In some embodiments, the immunogenic fragment has only 3 amino acids from the wild-type amino acid sequence of ASTE1 (SEQ ID NO: 4), and these are at the N-terminus of the immunogenic fragment. In some embodiments, the peptide has only 3 amino acids from the wild-type amino acid sequence of ASTE1 (SEQ ID NO: 4), and these are at the N-terminus of the peptide.

In some embodiments, the immunogenic fragment of mutASTE1 (SEQ ID NO: 5) comprises position 1 to position 8, position 2 to position 9, position 3 to position 10, position 4 to position 11, position 5 to position 12, position 6 to position 13, position 7 to position 14, position 8 to position 15, position 9 to position 16, position 10 to position 17, position 11 to position 18, position 12 to position 19, position 13 to position 20, position 14 to position 21, position 15 to position 22, position 16 to position 23, position 17 to position 24 or position 18 to position 25 of SEQ ID NO: 26.

In some embodiments, the immunogenic fragment of mutASTE1 (SEQ ID NO: 5) comprises position 1 to position 9 or position 8 to position 16 of SEQ ID NO: 26.

In some embodiments, the immunogenic fragment of mutASTE1 (SEQ ID NO: 5) comprises position 1 to position 10, position 2 to position 11, position 3 to position 12, position 4 to position 13, position 5 to position 14, position 6 to position 15, position 7 to position 16, position 8 to position 17, position 9 to position 18, position 10 to position 19, position 11 to position 20, position 12 to position 21, position 13 to position 22, position 14 to position 23, position 15 to position 24 or position 16 to position 25 of SEQ ID NO: 26.

In some embodiments, the immunogenic fragment of mutASTE1 (SEQ ID NO: 5) comprises position 1 to position 15, position 2 to position 16, position 3 to position 17, position 4 to position 18, position 5 to position 19, position 6 to position 20, position 7 to position 21, position 8 to position 22, position 9 to position 23, position 9 to position 24 or position 10 to position 25 of SEQ ID NO: 26.

In some embodiments, the immunogenic fragment of mutASTE1 (SEQ ID NO: 5) comprises position 1 to position 8, position 1 to position 9 or position 1 to position 15 of SEQ ID NO: 26, and the immunogenic fragment is the N-terminus of the peptide. In some embodiments, the immunogenic fragment comprises position 4 to position 11 or position 4 to position 18 of SEQ ID NO: 26, and the immunogenic fragment starts at position four from the N-terminus of the peptide. In some embodiments, the immunogenic fragment comprises position 8 to position 15 or position 8 to position 16 of SEQ ID NO: 26, and the immunogenic fragment starts at position eight from the N-terminus of the peptide. In some embodiments, the immunogenic fragment comprises position 9 to position 16 or position 9 to position 23 of SEQ ID NO: 26, and the fragment starts at position nine from the N-terminus of the peptide. In some embodiments, the immunogenic fragment comprises position 11 to position 18 or position 11 to position 25 of SEQ ID NO: 26, and the immunogenic fragment ends at position eight from the C-terminus of the peptide.

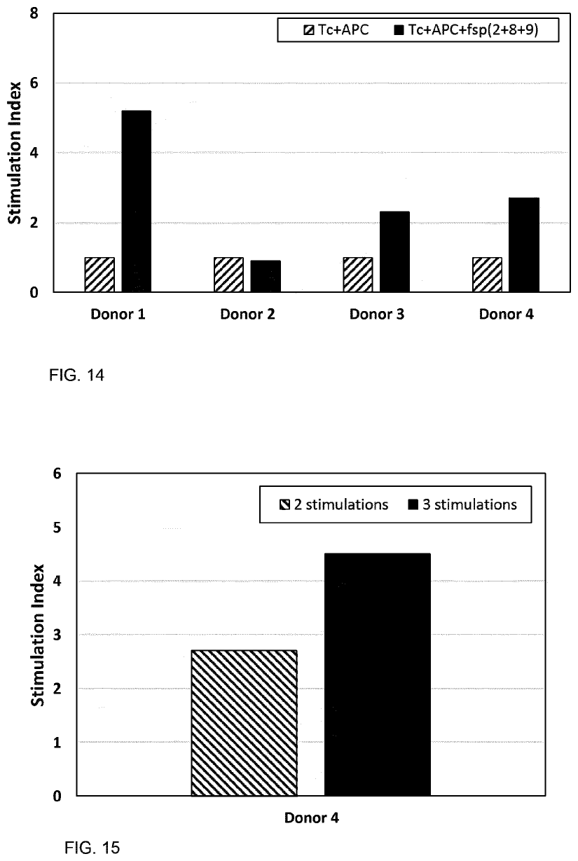

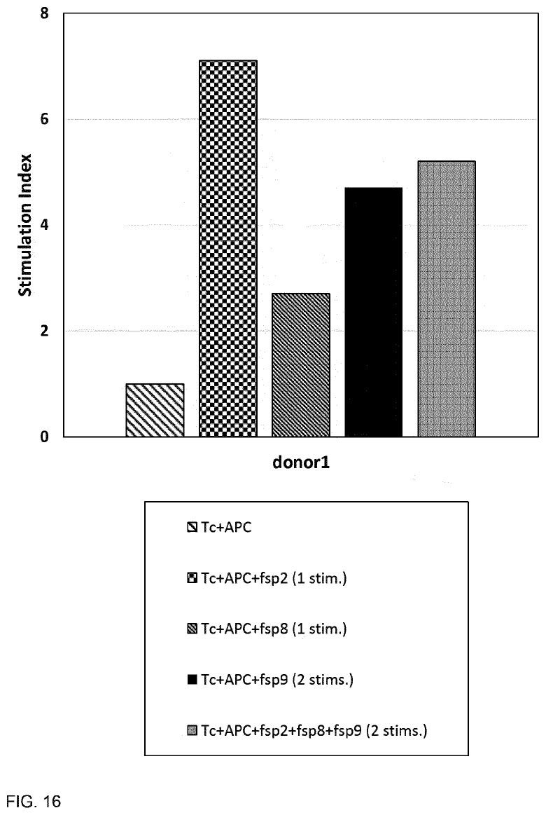

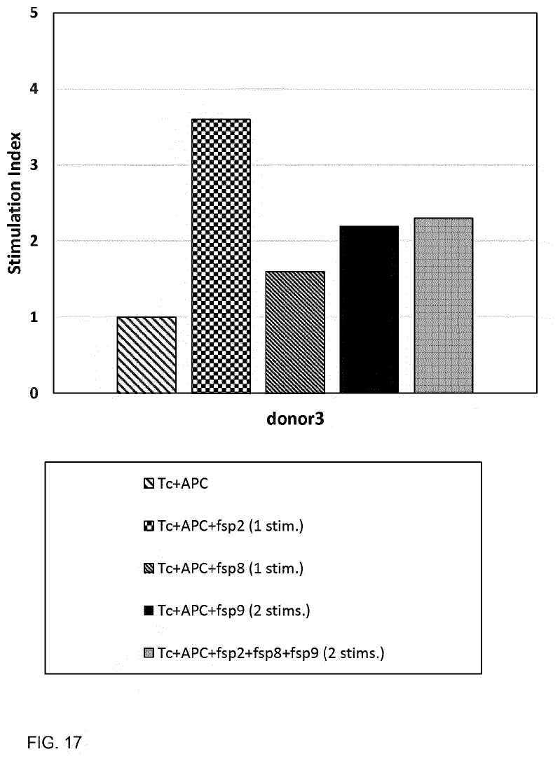

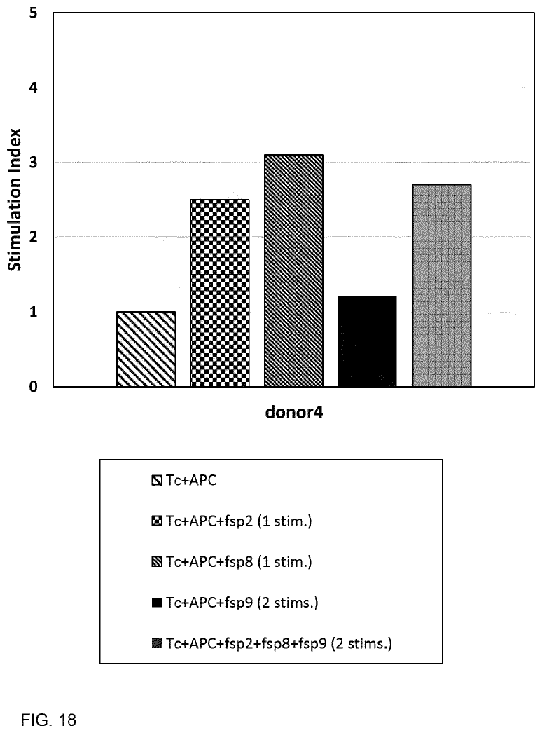

In some embodiments, the immunogenic fragment comprises the amino acid sequence of SEQ ID NO: 26. In some embodiments, the immunogenic fragment consists of the amino acid sequence of SEQ ID NO: 26. In other embodiments, the peptide capable of inducing an immune response against mutASTE1 (SEQ ID NO: 5) consists of the amino acid sequence of SEQ ID NO: 26. The peptide consisting of the sequence of SEQ ID NO: 26 is referred to herein as “fsp8”. In particular, FIG. 14 shows that a peptide mixture containing fsp8 is immunogenic, as the peptide mixture induces a T-cell response in three out of four donors. In addition, FIGS. 16-18 show that fsp8 alone induces a T-cell response even after only one round of stimulation with a peptide mixture containing fsp8. Thus, fsp8 is immunogenic.

In some embodiments, the immunogenic fragment of mutASTE1 (SEQ ID NO: 5) comprises a glycine residue at the C-terminus thereof. In some embodiments, the peptide capable of inducing an immune response against mutASTE1 (SEQ ID NO: 5) comprises a glycine residue at the C-terminus thereof. FIGS. 21 and 22 show that a peptide of mutASTE1 (SEQ ID NO: 5), having a glycine residue that the C-terminus thereof, is capable of inducing T-cell response whether administered as a single peptide composition or as part of a peptide mixture.

In some embodiments, the immunogenic fragment of mutASTE1 (SEQ ID NO: 5) or the peptide capable of inducing an immune response against mutASTE1 (SEQ ID NO: 5) comprises 3 amino acids from the wild-type amino acid sequence of ASTE1 (SEQ ID NO: 4) at the N-terminus thereof, and a glycine residue at the C-terminus thereof. In some embodiments, the immunogenic fragment or the peptide has only 3 amino acids from the wild-type amino acid sequence of ASTE1 (SEQ ID NO: 4), which are at the N-terminus of the immunogenic fragment or peptide, and the immunogenic fragment or peptide has a glycine residue at the C-terminus thereof. In some embodiments, the immunogenic fragment or the peptide does not comprise any amino acids from the wild-type amino acid sequence of ASTE1 (SEQ ID NO: 4), and the immunogenic fragment or peptide has a glycine residue at the C-terminus thereof.

In some embodiments, the immunogenic fragment of mutASTE1 (SEQ ID NO: 5) consists of the amino acid sequence of SEQ ID NO: 127. In some embodiments, the peptide capable of inducing an immune response against mutASTE1 (SEQ ID NO: 5) consists of the amino acid sequence of SEQ ID NO: 127. The peptide consisting of the amino acid sequence of SEQ ID NO: 127 is referred to herein as “fsp15”. In particular, and as mentioned above, FIGS. 21 and 22 show that a peptide mixture containing fsp15, and fsp15 alone, is immunogenic, as the peptide mixture and the peptide alone induce a T-cell response. Thus, fsp15 is immunogenic.

TAF1β Peptide

In some embodiments, in the peptide capable of inducing an immune response against a TAF1β-1a frameshift mutant protein (mutTAF1β; SEQ ID NO: 7), the immunogenic fragment comprises at least 8 consecutive amino acids of SEQ ID NO: 27. In some embodiments, the immunogenic fragment comprises at least 9 consecutive amino acids of SEQ ID NO: 27. In some embodiments, the immunogenic fragment comprises at least 10 consecutive amino acids of SEQ ID NO: 27. In some embodiments, the immunogenic fragment comprises at least 12 consecutive amino acids of SEQ ID NO: 27. In some embodiments, the immunogenic fragment comprises at least 15 consecutive amino acids of SEQ ID NO: 27. In some embodiments, the immunogenic fragment comprises at least 20 of SEQ ID NO: 27. In other embodiments, the immunogenic fragment comprises at least 25 amino acids of SEQ ID NO: 27.

In some embodiments, the immunogenic fragment comprises no more than 25 amino acids.

In some embodiments, the peptide comprises at least 10 amino acids. In some embodiments, the peptide comprises at least 12 amino acids. In some embodiments, the peptide comprises at least 15 amino acids. In some embodiments, the peptide comprises 20 amino acids. In other embodiments, the peptide comprises at least 25 amino acids.

In some embodiments, the peptide comprises no more than 25 amino acids.

In some embodiments, the immunogenic fragment of mutTAF1β (SEQ ID NO: 7) comprises at least one amino acid from the wild-type amino acid sequence of TAF1β (SEQ ID NO: 6) (i.e. position 1 to position 65 of SEQ ID NO: 7) consecutive with at least one amino acid from the amino acid sequence resulting from the frameshift mutation (position 66 to position 90 of SEQ ID NO: 7). Thus, the immunogenic fragment may be a fragment of mutTAF1β (SEQ ID NO: 7) which overlaps the amino acid sequence unaffected by the frameshift mutation and the amino acid sequence resulting from the frameshift mutation. In some embodiments, the immunogenic fragment comprises two, three, four, five, six or seven consecutive amino acids from the wild-type amino acid sequence of TAF1β (SEQ ID NO: 6). In some embodiments, the at least one amino acid from the wild-type sequence of TAF1β (SEQ ID NO: 6) is consecutive with 1, 2, 3, 4, 5, 6, 7, 8, 9, 10, 11, 12, 13, 14, 15, 16, 17, 18, 19, 20, 21, 22, 23, 24 or 25 amino acids from the amino acid sequence resulting from the frameshift mutation.

In some embodiments, the immunogenic fragment of mutTAF1β (SEQ ID NO: 7) comprises no more than 8 amino acids from the wild-type amino acid sequence of TAF1β (SEQ ID NO: 6). In some embodiments, the immunogenic fragment of mutTAF1β (SEQ ID NO: 7) comprises no more than 7, no more than 5, no more than 3, no more than 2 or no more than 1 amino acids from the wild-type amino acid sequence of TAF1β (SEQ ID NO: 6). In some embodiments, the immunogenic fragment has only 5 amino acids from the wild-type amino acid sequence of TAF1β (SEQ ID NO: 6). In some embodiments, the immunogenic fragment has only 1 amino acid from the wild-type amino acid sequence of TAF1β (SEQ ID NO: 6).

In some embodiments, the peptide comprises no more than 8 amino acids from the wild-type amino acid sequence of TAF1β (SEQ ID NO: 6). In some embodiments, the peptide comprises no more than 7, no more than 5, no more than 3, no more than 2 or no more than 1 amino acids from the wild-type amino acid sequence of TAF1β (SEQ ID NO: 6). In some embodiments, the peptide has only 5 amino acids from the wild-type amino acid sequence of TAF1β (SEQ ID NO: 6). In some embodiments, the peptide has only 1 amino acid from the wild-type amino acid sequence of TAF1β (SEQ ID NO: 6).

When an immunogenic fragment or a peptide comprises one or more amino acids from the wild-type sequence of TAF1β (SEQ ID NO: 6), these amino acids are preferably at the N-terminus of the immunogenic fragment or peptide. In some embodiments, the immunogenic fragment has only 5 amino acids from the wild-type amino acid sequence of TAF1β (SEQ ID NO: 6), and these are at the N-terminus of the immunogenic fragment. In some embodiments, the peptide has only 5 amino acids from the wild-type amino acid sequence of TAF1β (SEQ ID NO: 6), and these are at the N-terminus of the peptide. In some embodiments, immunogenic fragment has only 1 amino acid from the wild-type amino acid sequence of TAF1β (SEQ ID NO: 6), and this is at the N-terminus of the immunogenic fragment. In some embodiments, the peptide has only 1 amino acid from the wild-type amino acid sequence of TAF1β (SEQ ID NO: 6), and this is at the N-terminus of the peptide.

In some embodiments, the immunogenic fragment of mutTAF1β (SEQ ID NO: 7) comprises positions 65 and 66 of SEQ ID NO: 7, wherein the amino acid at position 65 of SEQ ID NO: 7 corresponds to the amino acid at position 65 of wild-type TAF1β (SEQ ID NO: 6) and the amino acid at position 66 of SEQ ID NO: 7 is the first amino acid of the amino acid sequence resulting from the frameshift mutation in mutTAF1β (SEQ ID NO: 7). In some embodiments, the immunogenic fragment comprises position 64 to position 66, position 63 to position 66, position 62 to position 66, position 61 to position 66, position 60 to position 66 or position 59 to position 66 of SEQ ID NO: 7. In some embodiments, the immunogenic fragment or the peptide has only one amino acid from the wild-type amino acid sequence of TAF1β (SEQ ID NO: 6). In some embodiments, the one amino acid from the wild-type amino acid sequence of TAF1β (SEQ ID NO: 6) is position 65 of SEQ ID NO: 6 (i.e. position 65 of SEQ ID NO: 7). In particular, FIG. 14 shows that a peptide mixture comprising such a peptide or immunogenic fragment (i.e. fsp9; SEQ ID NO: 27) induces a T-cell response, and FIG. 16 shows that such a peptide or immunogenic fragment (i.e. fsp9; SEQ ID NO: 27), alone, induces a T-cell response. Thus, an immunogenic fragment or peptide having only one amino acid from the wild-type amino acid sequence of TAF1β (SEQ ID NO: 6), preferably which is position 65 of SEQ ID NO: 6, is immunogenic.

In some embodiments, the immunogenic fragment or the peptide has only 5 amino acids from the wild-type sequence of TAF1β (SEQ ID NO: 6), and these amino acids are position 61 to position 65 of SEQ ID NO: 6 (i.e. position 61 to position 65 of SEQ ID NO: 7). FIG. 22 shows that a peptide mixture containing such an immunogenic fragment or peptide induces a T-cell response and, therefore, is immunogenic.

In particular, the predicted nested epitope identified by the predicted nested epitope found by SYFPEITHI algorithm used in Example 6 (see Table 11) contains seven amino acids from the wild-type sequence of TAF1β (SEQ ID NO: 6) as well as amino acids from the amino acid sequence resulting from the frameshift mutation in TAF1β (SEQ ID NO: 6). In addition, several of the HLA class-II predicted epitopes shown in Table 11 contain one or more amino acids from the wild-type sequence of TAF1@R (SEQ ID NO: 6). Therefore, it is expected that the amino acid residues bridging the wild-type sequence of TAF1β (SEQ ID NO: 6) and the amino acid sequence resulting from the frameshift mutation are important for some immunogenic epitopes of mutTAF1@ (SEQ ID NO: 7).

In some embodiments, the immunogenic fragment of mutTAF1β (SEQ ID NO: 7) comprises position 1 to position 8, position 2 to 9, position 3 to position 10, position 4 to position 11, position 5 to position 12, position 6 to position 13, position 7 to position 14, position 8 to position 15, position 9 to position 16, position 10 to position 17, position 11 to position 18, position 12 to position 19, position 13 to position 20, position 14 to position 21, position 15 to position 22, position 16 to position 23, position 17 to position 24 or position 18 to position 25 of SEQ ID NO: 27.

In some embodiments, the immunogenic fragment of mutTAF1β (SEQ ID NO: 7) comprises position 1 to position 9, position 6 to position 14, position 15 to position 23 or position 17 to position 25 of SEQ ID NO: 27.

In some embodiments, the immunogenic fragment of mutTAF1β (SEQ ID NO: 7) comprises position 1 to position 10, position 2 to position 11, position 3 to position 12, position 4 to position 13, position 5 to position 14, position 6 to position 15, position 7 to position 16, position 8 to position 17, position 9 to position 18, position 10 to position 19, position 11 to position 20, position 12 to position 21, position 13 to position 22, position 14 to position 23, position 15 to position 24 or position 16 to position 25 of SEQ ID NO: 27.

In some embodiments, the immunogenic fragment of mutTAF1β (SEQ ID NO: 7) comprises position 1 to position 13, position 2 to position 14, position 3 to position 15, position 4 to position 16, position 5 to position 17, position 6 to position 18, position 6 to position 19, position 7 to position 20, position 8 to position 21, position 9 to position 22, position 10 to position 23, position 11 to position 24 or position 12 to position 25 of SEQ ID NO: 27.

In some embodiments, the immunogenic fragment of mutTAF1β (SEQ ID NO: 7) comprises position 6 to position 20, position 8 to position 22, position 9 to position 23 or position 10 to position 24 of SEQ ID NO: 27.

In some embodiments, the immunogenic fragment of mutTAF1β (SEQ ID NO: 7) comprises position 1 to position 8 or position 1 to position 9 of SEQ ID NO: 27, and the immunogenic fragment starts at position 1 of the peptide. In some embodiments, the immunogenic fragment of mutTAF1β (SEQ ID NO: 7) comprises position 2 to position 9 or position 2 to position 14 of SEQ ID NO: 27, and the immunogenic fragment starts at position two from the N-terminus of the peptide. In some embodiments, the immunogenic fragment comprises position 6 to position 13, position 6 to position 14 or position 6 to position 20 of SEQ ID NO: 27, and the immunogenic fragment starts at position six from the N-terminus of the peptide. In some embodiments, the immunogenic fragment comprises position 8 to position 15 or position 8 to position 22 of SEQ ID NO: 27, and the immunogenic fragment starts at position eight from the N-terminus of the peptide. In some embodiments, the immunogenic fragment comprises position 9 to position 16 or position 9 to position 23 of SEQ ID NO: 27, and the immunogenic fragment starts at position nine from the N-terminus of the peptide. In some embodiments, the immunogenic fragment comprises position 10 to position 18 or position 10 to position 24 of SEQ ID NO: 27, and the immunogenic fragment ends at position nine from the C-terminus of the peptide. In some embodiments, the immunogenic fragment comprises position 15 to position 22 of SEQ ID NO: 27, and the immunogenic fragment ends at position three from the C-terminus of the peptide. In some embodiments, the immunogenic fragment comprises position 15 to position 23 of SEQ ID NO: 27, and the immunogenic fragment ends at position two from the C-terminus of the peptide. In some embodiments, the immunogenic fragment comprises position 17 to position 24 of SEQ ID NO: 27, and the immunogenic fragment ends at position two from the C-terminus of the peptide. In other embodiments, the immunogenic fragment comprises position 17 to position 25 of SEQ ID NO: 27, and the immunogenic fragment is the C-terminus of the peptide.

In some embodiments, the immunogenic fragment of mutTAF1β (SEQ ID NO: 7) comprises the amino acid sequence of SEQ ID NO: 27. In some embodiments, the immunogenic fragment consists of the amino acid sequence of SEQ ID NO: 27. In other embodiments, the peptide capable of inducing an immune response against mutTAF1β (SEQ ID NO: 7) consists of the amino acid sequence of SEQ ID NO: 27. The peptide consisting of the sequence of SEQ ID NO: 27 is referred to herein as “fsp9”. In particular, FIG. 14 shows that a peptide mixture containing fsp9 is immunogenic, as the peptide mixture induces a T-cell response in three out of four donors. In addition, FIGS. 16-18 show that fsp9 alone induces a T-cell response even after only one round of stimulation with a peptide mixture containing fsp9. Thus, fsp9 is immunogenic.

In some embodiments, the immunogenic fragment of mutTAF1β (SEQ ID NO: 7) comprises a glycine residue at the C-terminus thereof. In some embodiments, the peptide capable of inducing an immune response against mutTAF1β (SEQ ID NO: 7) comprises a glycine residue at the C-terminus thereof. FIG. 22 shows that a peptide of mutTAF1β (SEQ ID NO: 7), having a glycine residue that the C-terminus thereof, is capable of inducing T-cell response whether administered as part of a peptide mixture.

In some embodiments, the immunogenic fragment of mutTAF1β (SEQ ID NO: 7) or the peptide capable of inducing an immune response against mutTAF1β (SEQ ID NO: 7) comprises 5 amino acids from the wild-type amino acid sequence of TAF1β (SEQ ID NO: 6) at the N-terminus thereof, and a glycine residue at the C-terminus thereof. In some embodiments, the immunogenic fragment or the peptide comprises 1 amino acid from the wild-type amino acid sequence of TAF1β (SEQ ID NO: 6), and comprises a glycine residue at the C-terminus thereof. In some embodiments, the immunogenic fragment or the peptide has only 5 or has only 1 amino acid from the wild-type sequence of TAF1β (SEQ ID NO: 6), and the 5 or 1 amino acids are at the N-terminus of the immunogenic fragment or peptide, and the immunogenic fragment or peptide has a glycine residue at the C-terminus thereof.

In some embodiments, the immunogenic fragment of mutTAF1β (SEQ ID NO: 7) consists of the amino acid sequence of SEQ ID NO: 128. In some embodiments, the peptide capable of inducing an immune response against mutTAF1β (SEQ ID NO: 7) consists of the amino acid sequence of SEQ ID NO: 128. The peptide consisting of the amino acid sequence of SEQ ID NO: 128 is referred to herein as “fsp16”. In particular, FIG. 22 shows that a peptide mixture containing fsp16 is immunogenic, as the peptide mixture induces a T-cell response.

KIAA2018 Peptides

In some embodiments, in the peptide capable of inducing an immune response against a KIAA2018-1a frameshift mutant protein (mutKIAA2018; SEQ ID NOs: 9-12), the immunogenic fragment comprises at least 8 consecutive amino acids of one of SEQ ID NOs: 9-12, wherein the immunogenic fragment comprises at least one amino acid from positions 13 to 37 of SEQ ID NO: 9, positions 91 to 109 of SEQ ID NO: 10, positions 147 to 167 of SEQ ID NO: 11 and positions 1016 to 1037 of SEQ ID NO: 12, respectively In some embodiments, the immunogenic fragment comprises at least 9 consecutive amino acids of one of SEQ ID NOs: 9-12, wherein the immunogenic fragment includes at least one amino acid from positions 13 to 37 of SEQ ID NO: 9, position 91-109 of SEQ ID NO: 10, positions 147 to 167 of SEQ ID NO: 11 and positions 1016 to 1037 of SEQ ID NO: 12. In some embodiments, the immunogenic fragment comprises at least 10 consecutive amino acids of one of SEQ ID NOs: 9-12, wherein the immunogenic fragment includes at least one amino acid from positions 13 to 37 of SEQ ID NO: 9, position 91-109 of SEQ ID NO: 10, positions 147 to 167 of SEQ ID NO: 11 and positions 1016 to 1037 of SEQ ID NO: 12. In some embodiments, the immunogenic fragment comprises at least 12 consecutive amino acids of one of SEQ ID NOs: 9-12, wherein the immunogenic fragment includes at least one amino acid from positions 13 to 37 of SEQ ID NO: 9, position 91-109 of SEQ ID NO: 10, positions 147 to 167 of SEQ ID NO: 11 and positions 1016 to 1037 of SEQ ID NO: 12. In some embodiments, the immunogenic fragment comprises at least 15 consecutive amino acids of one of SEQ ID NOs: 9-12, wherein the immunogenic fragment includes at least one amino acid from positions 13 to 37 of SEQ ID NO: 9, position 91-109 of SEQ ID NO: 10, positions 147 to 167 of SEQ ID NO: 11 and positions 1016 to 1037 of SEQ ID NO: 12. In some embodiments, the immunogenic fragment comprises at least 20 consecutive amino acids of one of SEQ ID NOs: 9-12, wherein the immunogenic fragment includes at least one amino acid from positions 13 to 37 of SEQ ID NO: 9, position 91-109 of SEQ ID NO: 10, positions 147 to 167 of SEQ ID NO: 11 and positions 1016 to 1037 of SEQ ID NO: 12. In other embodiments, the immunogenic fragment comprises at least 24 amino acids of one of SEQ ID NOs: 9-12, wherein the immunogenic fragment includes at least one amino acid from positions 13 to 37 of SEQ ID NO: 9, position 91-109 of SEQ ID NO: 10, positions 147 to 167 of SEQ ID NO: 11 and positions 1016 to 1037 of SEQ ID NO: 12 In some embodiments, the immunogenic fragment comprises no more than 24 consecutive amino acids of one of SEQ ID NOs: 9-12.

In some embodiments, the immunogenic fragment of mutKIAA2018 (SEQ ID NOs: 9-12) comprises at least one peptide from the wild-type amino acid sequence of KIAA2018 (SEQ ID NO: 8) (i.e. position 1 to position 12 of SEQ ID NO: 9, position 1 to position 90 of SEQ ID NO: 10, position 1 to position 146 of SEQ ID NO: 11 or position 1 to position 1015 of SEQ ID NO: 12) consecutive with at least one amino acid from the amino acid sequence resulting from the frameshift mutation (position 13 to position 37 of SEQ ID NO: 9, position 91 to position 109 of SEQ ID NO: 10, position 127 to position 167 of SEQ ID NO: 11, or position 1017 to position 1037 of SEQ ID NO: 12). Thus, the immunogenic fragment may be a fragment of mutKIAA2018 (SEQ ID NOs: 9-12) which overlaps the amino acid sequence unaffected by the frameshift mutation and the amino acid sequence resulting from the frameshift mutation. In some embodiments, the immunogenic fragment comprises two consecutive amino acids from the wild-type amino acid sequence of KIAA2018 (SEQ ID NO: 8). In some embodiments, the at least one amino acid from the wild-type sequence of KIAA2018 (SEQ ID NO: 8) is consecutive with 1, 2, 3, 4, 5, 6, 7, 8, 9, 10, 11, 12, 13, 14, 15, 16, 17, 18, 19, 20, 21 or 22 amino acids from the amino acid sequence resulting from the frameshift mutation.

In some embodiments, the immunogenic fragment of mutKIAA2018 (SEQ ID NOs: 9-12), or the peptide capable of inducing an immune response against mutKIAA2018 (SEQ ID NOs: 9-12) comprises no more than 8 amino acids from the wild-type sequence of KIAA2018 (SEQ ID NO: 8). In some embodiments, the immunogenic fragment or the peptide comprises no more than 3, no more than 2 or no more than 1 amino acid from the wild-type amino acid sequence of KIAA2018 (SEQ ID NO: 8). In some embodiments, the immunogenic fragment or the peptide has only 2 amino acids from the wild-type amino acid sequence of KIAA2018 (SEQ ID NO: 8).

In some embodiments, the immunogenic fragment of mutKIAA2018 (SEQ ID NOs: 9-12) comprises positions 1015 and 1016 of SEQ ID NO: 12, wherein the amino acid at position 1015 of SEQ ID NO: 12 corresponds to the amino acid at position 1015 of wild-type KIAA2018 (SEQ ID NO: 8) and the amino acid at position 1016 of SEQ ID NO: 12 is the first amino acid of the amino acid sequence resulting from the frameshift mutation in mutKIAA2018(pos1016). In some embodiments, the immunogenic fragment comprises position 1014 to position 1016 of SEQ ID NO: 12. In some embodiments, the immunogenic fragment or the peptide has only 2 amino acids from the wild-type amino acid sequence of KIAA2018 (SEQ ID NO: 8), and these are positions 1014 and 1015 of SEQ ID NO: 8 (which correspond to positions 1014 and 1015 of SEQ ID NO: 12).

In particular, the predicted nested epitope identified by the predicted nested epitope found by SYFPEITHI algorithm used in Example 6 (see Table 12) contains two amino acids from the wild-type sequence of KIAA2018 (SEQ ID NO: 8) as well as amino acids from the amino acid sequence resulting from a frameshift mutation in KIAA2018. In addition, one of the HLA class-II predicted epitopes shown in Table 12 contains two amino acids from the wild-type sequence of KIAA2018 (SEQ ID NO: 8). Therefore, it is expected that the amino acid residues bridging the wild-type sequence of KIAA2018 (SEQ ID NO: 8) and the amino acid sequence resulting from the frameshift mutation are important for some immunogenic epitopes of mutKIAA2018 (SEQ ID NO: 9-12), and particularly for some immunogenic epitopes of mutKIAA2018(pos1016) (SEQ ID NO: 12).

When an immunogenic fragment of mutKIAA2018 (SEQ ID NOS: 9-12), or a peptide capable of inducing an immune response against mutKIAA2018 (SEQ ID NOS: 9-12), comprises one or more amino acids from the wild-type amino acid sequence of KIAA2018 (SEQ ID NO: 8), these amino acids are preferably at the N-terminus of the immunogenic fragment or peptide. In some embodiments, the immunogenic fragment or the peptide has only 2 amino acids from the wild-type amino acid sequence of KIAA2018 (SEQ ID NO: 8), and these are at the N-terminus of the immunogenic fragment or peptide, respectively.

In some embodiments, the peptide capable of inducing an immune response against a KIAA2018-1a frameshift mutant protein (mutKIAA2018; SEQ ID NOs: 9-12) comprises at least 9 amino acids. In some embodiments, the peptide comprises at least 10 amino acids. In some embodiments, the peptide comprises at least 12 amino acids. In some embodiments, the peptide comprises at least 15 amino acids. In some embodiments, the peptide comprises 20 amino acids. In other embodiments, the peptide comprises at least 24 amino acids.

In some embodiments, the peptide capable of inducing an immune response against a KIAA2018-1a frameshift mutant protein (mutKIAA2018; SEQ ID NOs: 9-12) comprises no more than 40 amino acids. In some embodiments, the peptide comprises no more than 24 amino acids.

In some embodiments, the immunogenic fragment of mutKIAA2018 (SEQ ID NOs: 9-12) comprises position 1 to position 8, position 2 to position 9, position 3 to position 10, position 4 to position 11, position 5 to position 12, position 6 to position 13, position 7 to position 14, position 8 to position 15, position 9 to position 16, position 10 to position 17, position 11 to position 18, position 12 to position 19, position 13 to position 20, position 14 to position 21, position 15 to position 22, position 16 to position 23 or position 17 to position 24 of SEQ ID NO: 28.

In some embodiments, the immunogenic fragment of mutKIAA2018 (SEQ ID NOs: 9-12) comprises position 3 to position 11, position 9 to position 17, position 13 to position 21, position 14 to position 22 or position 16 to position 24 of SEQ ID NO: 28.

In some embodiments, the immunogenic fragment of mutKIAA2018 (SEQ ID NOs: 9-12) comprises position 1 to position 10, position 2 to position 11, position 3 to position 12, position 4 to position 13, position 5 to position 14, position 6 to position 15, position 7 to position 16, position 8 to position 17, position 9 to position 18, position 10 to position 19, position 11 to position 20, position 12 to position 21, position 13 to position 22, position 14 to position 23 or position 15 to position 24 of SEQ ID NO: 28.

In some embodiments, the immunogenic fragment of mutKIAA2018 (SEQ ID NOs: 9-12) comprises position 1 to position 15, position 2 to position 16, position 3 to position 17, position 4 to position 18, position 5 to position 19, position 6 to position 20, position 7 to position 21, position 8 to position 22, position 9 to position 23, position 9 to position 24 of SEQ ID NO: 28.

In some embodiments, the immunogenic fragment of mutKIAA2018 (SEQ ID NOs: 9-12) comprises position 1 to position 8 or position 1 to position 15 of SEQ ID NO: 28, and the immunogenic fragment is the N-terminus of the peptide. In some embodiments, the immunogenic fragment comprises position 3 to position 10 or position 3 to position 11 of SEQ ID NO: 28, and the immunogenic fragment starts at position three from the N-terminus of the peptide. In some embodiments, the immunogenic fragment comprises position 5 to position 12 or position 5 to position 19 of SEQ ID NO: 28, and the immunogenic fragment starts at position five from the N-terminus of the peptide. In some embodiments, the immunogenic fragment comprises position 7 to position 14 or position 7 to position 21 of SEQ ID NO: 28, and the immunogenic fragment starts at position seven from the N-terminus of the peptide. In some embodiments, the immunogenic fragment comprises position 8 to position 15 or position 8 to position 22 of SEQ ID NO: 28, and the immunogenic fragment starts from position eight from the N-terminus of the peptide. In some embodiments, the immunogenic fragment comprises position 9 to position 16 or position 9 to position 17 of SEQ ID NO: 28, and the immunogenic fragment starts at position nine from the N-terminus of the peptide. In some embodiments, the immunogenic fragment comprises position 10 to position 17 of SEQ ID NO: 28, and the immunogenic fragment ends at positon eight from the C-terminus of the peptide. In some embodiments, the immunogenic fragment comprises position 16 to position 24 or position 10 to position 24, and the immunogenic fragment is the C-terminus of the peptide. In some embodiments, the immunogenic fragment comprises position 13 to position 20 of SEQ ID NO: 28, and the immunogenic fragment ends at position five from the C-terminus of the peptide. In some embodiments, the immunogenic fragment comprises position 14 to position 21 or position 13 to position 21 of SEQ ID NO: 28, and the immunogenic fragment ends at position four from the C-terminus of the peptide. In some embodiments, the immunogenic fragment comprises position 14 to position 22 of SEQ ID NO: 28, and the immunogenic fragment ends at position three from the C-terminus of the peptide. In other embodiments, the immunogenic fragment comprises position 16 to position 23 of SEQ ID NO: 28, and the immunogenic fragment ends at position two from the C-terminus of the peptide.

In some embodiments, the immunogenic fragment comprises the amino acid sequence of SEQ ID NO: 28. In some embodiments, the immunogenic fragment consists of the amino acid sequence of SEQ ID NO: 28. In other embodiments, the peptide capable of inducing an immune response against mutKIAA2018 (SEQ ID NOs: 9-12) consists of the amino acid sequence of SEQ ID NO: 28. The peptide consisting of the sequence of SEQ ID NO: 28 is referred to herein as “fsp10”. FIG. 20 shows that a peptide mixture containing fsp10 (SEQ ID NO: 28) is immunogenic, at both a low dose (3.3 μM per peptide) and a high does (10 μM per peptide).

In some embodiments, the immunogenic fragment of mutKIAA2018 (SEQ ID NOs: 9-12), or the peptide capable of inducing an immune response against mutKIAA2018 (SEQ ID NOs: 9-12), comprises a glycine residue at the C-terminus thereof.

SLC22A9 Peptides

In some embodiments, in the peptide capable of inducing an immune response against a SCL22A9-1a frameshift mutant protein (SEQ ID NOs: 14-16), the immunogenic fragment comprises at least 8 consecutive amino acids of one of SEQ ID NOs: 14-18, wherein the immunogenic fragment comprises at least one amino acid from positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18, respectively. In some embodiments, the immunogenic fragment comprises at least 9 consecutive amino acids of one of SEQ ID NOs: 14-18, wherein the immunogenic fragment comprises at least one amino acids positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18. In some embodiments, In some embodiments, the immunogenic fragment comprises at least 10 consecutive amino acids of one of SEQ ID NOs: 14-18, wherein the immunogenic fragment comprises at least one amino acids positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18. In some embodiments, the immunogenic fragment comprises at least 12 amino acids of one of SEQ ID NOs: 14-18, wherein the immunogenic fragment comprises at least one amino acids positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18. In some embodiments, the immunogenic fragment comprises at least 15 consecutive amino acids of one of SEQ ID NOs: 14-18, wherein the immunogenic fragment comprises at least one amino acids positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18. In some embodiments, the immunogenic fragment comprises at least 20 consecutive amino acids of one of SEQ ID NOs: 14-18, wherein the immunogenic fragment comprises at least one amino acids positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18. In some embodiments, the immunogenic fragment comprises at least 25 consecutive amino acids of one of SEQ ID NOs: 14-18, wherein the immunogenic fragment comprises at least one amino acids positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18. In other embodiments, the immunogenic fragment comprises at least 28 consecutive amino acids of one of SEQ ID NOs: 14-18, wherein the immunogenic fragment comprises at least one amino acids positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18.

In some embodiments, the immunogenic fragment of mutSLC22A9 (SEQ ID NOs: 14-16) comprises no more than 28 amino acids.

In some embodiments, the peptide capable of inducing an immune response against mutSLC22A9 (SEQ ID NOs: 14-16) comprises at least 8 amino acids. In some embodiments, the peptide comprises at least 9 amino acids. In some embodiments, the peptide comprises at least 10 amino acids. In some embodiments, the peptide comprises at least 12 amino acids. In some embodiments, the peptide comprises at least 15 amino acids. In some embodiments, the peptide comprises at least 20 amino acids. In some embodiments, the peptide comprises at least 25 amino acids. In some embodiments, the peptide comprises at least 28 amino acids.

In some embodiments, the peptide capable of inducing an immune response against mutSLC22A9 (SEQ ID NOs: 14-16) comprises no more than 40 amino acids. In other embodiments, the peptide comprises no more than 28 amino acids.

In some embodiments, the immunogenic fragment of mutSLC22A9 (SEQ ID NOs: 14-16), or the peptide capable of inducing an immune response against mutSLC22A9 (SEQ ID NOs: 14-16), comprises no more than 8 amino acids from the wild-type amino acid sequence of SLC22A9 (SEQ ID NO: 13). In some embodiments, the immunogenic fragment or the peptide comprises no more than 3, no more than 2 or no more than 1 amino acid from the wild-type sequence of SLC22A9 (SEQ ID NO: 13). In some embodiments, the immunogenic fragment of or the peptide does not contain any amino acids from the wild-type amino acid sequence of SLC22A9 (SEQ ID NO: 13).

In some embodiments, the immunogenic fragment of mutSLC22A9 (SEQ ID NOs: 14-16), or the peptide capable of inducing an immune response against mutSLC22A9 (SEQ ID NOs: 14-16), comprises a glycine residue at the C-terminus thereof.

fsp11 and fsp13

In some embodiments, the immunogenic fragment of mutSLC22A9 (SEQ ID NOs: 14-16) comprises position 1 to position 8, position 4 to position 11, position 7 to position 14, position 8 to position 15, position 9 to position 16, position 10 to position 17, position 11 to position 18 or position 14 to position 21 of SEQ ID NO: 29, or position 1 to position 8, position 3 to position 10, position 4 to position 11 or position 10 to position 18 of SEQ ID NO: 31.

In some embodiments, the immunogenic fragment of mutSLC22A9 (SEQ ID NOs: 14-16) comprises position 8 to position 16, position 9 to position 17 or position 14 to position 22 of SEQ ID NO: 29, or position 10 to position 18 of SEQ ID NO: 31.

In some embodiments, the immunogenic fragment of mutSLC22A9 (SEQ ID NOs: 14-16) comprises position 1 to position 10, position 4 to position 13, position 7 to position 16, position 8 to position 17, position 10 to position 19 or position 11 to position 20 of SEQ ID NO: 29, or position 1 to position 10, position 3 to position 12 or position 4 to position 13 of SEQ ID NO: 31.

In some embodiments, the immunogenic fragment of mutSLC22A9 (SEQ ID NOs: 14-16) comprises position 1 to position 15, position 4 to position 19, position 7 to position 21, position 8 to position 22, position 10 to position 24 or position 11 to position 25 of SEQ ID NO: 29, or position 1 to position 15, position 3 to position 17 or position 4 to position 18 of SEQ ID NO: 31.

In some embodiments, the immunogenic fragment of mutSLC22A9 (SEQ ID NOs: 14-16) comprises position 1 to position 8 or position 1 to position 15 of SEQ ID NO: 29, and the immunogenic fragment is the N-terminus of the peptide. In some embodiments, the immunogenic fragment comprises position 4 to position 11 or position 4 to position 19 of SEQ ID NO: 29, and the fragment starts at positon four from the N-terminus of the peptides. In some embodiments, the immunogenic fragment comprises position 7 to position 14 or position 7 to position 21 of SEQ ID NO: 29, and the immunogenic fragment starts at position seven from the N-terminus of the peptide. In some embodiments, the immunogenic fragment comprises position 8 to position 15, position 8 to position 16 or position 8 to position 22 of SEQ ID NO: 29, and the immunogenic fragment starts at position eight from the N-terminus of the peptide. In some embodiments, the immunogenic fragment comprises position 9 to position 16 or position 9 to position 17 of SEQ ID NO: 29, and the immunogenic fragment starts at position nine from the N-terminus of the peptide. In some embodiments, the immunogenic fragment comprises position 10 to position 18 or position 10 to position 24 of SEQ ID NO: 29, and the immunogenic fragment starts at position ten of the peptide. In some embodiments, the immunogenic fragment comprises position 11 to position 18 or position 11 to position 25 of SEQ ID NO: 29, and the immunogenic fragment starts at position eleven from the N-terminus of the peptide. In some embodiments, the immunogenic fragment comprises position 14 to position 21 of SEQ ID NO: 29, and the immunogenic fragment ends at position eight from the C-terminus of the peptide. In some embodiments, the immunogenic fragment comprises position 14 to position 22 of SEQ ID NO: 29, and the immunogenic fragment ends at position seven from the C-terminus of the peptide.

In some embodiments, the immunogenic fragment of mutSLC22A9 (SEQ ID NOs: 14-16) comprises position 1 to position 8 or position 1 to position 15 of SEQ ID NO: 31, and the immunogenic fragment is the N-terminus of the peptide. In some embodiments, the immunogenic fragment comprises position 3 to position 10 or position 3 to position 17 of SEQ ID NO: 31, and the immunogenic fragment starts at position three from the N-terminus of the peptide. In some embodiments, the immunogenic fragment comprises position 4 to position 11 or position 4 to position 18 of SEQ ID NO: 31, and the immunogenic fragment starts at position four of the peptide. In some embodiments, the immunogenic fragment comprises position 10 to position 18 of SEQ ID NO: 31, and the immunogenic fragment starts at position ten from the N-terminus of the peptide.

In some embodiments, the immunogenic fragment of mutSLC22A9 (SEQ ID NOs: 14-16) does not contain any amino acids from the wild-type amino acid sequence of SLC22A9 (SEQ ID NO: 13).

In some embodiments, the immunogenic fragment of mutSLC22A9 (SEQ ID NOs: 14-16) comprises the amino acid sequence of SEQ ID NO: 29 or SEQ ID NO: 31. In some embodiments, the immunogenic fragment consists of the amino acid sequence of SEQ ID NO: 29 or SEQ ID NO: 31. In some embodiments, the peptide capable of inducing an immune response against mutSLC22A9 (SEQ ID NO: 14-16) consists of the amino acid sequence of SEQ ID NO: 29 or SEQ ID NO: 31. When the peptide consists of the amino acid sequence of SEQ ID NO: 29, the peptide is referred to herein as “fsp11”.

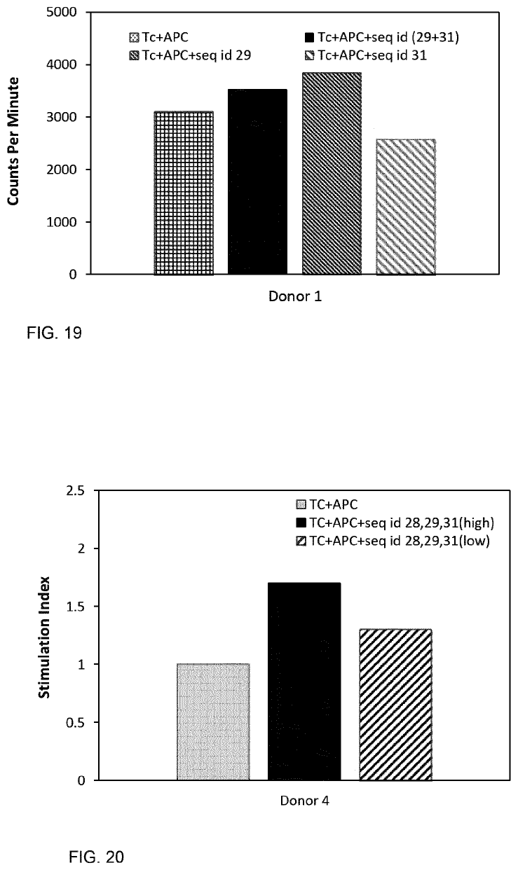

When the peptide consists of the amino acid sequence of SEQ ID NO: 31, the peptide is referred to herein as “fsp13”. FIG. 19 shows that fsp11 (SEQ ID NO: 29), alone or as part of a peptide mixture, is immunogenic, as a T-cell response was induced by both after only one round of stimulation. FIG. 19 also shows that a peptide mixture containing fsp13 (SEQ ID NO: 31) is immunogenic. Moreover, FIG. 20 shows that a peptide mixture containing both fsp11 and fsp13 is immunogenic at both a high dose (10 μM per peptide) and a low dose (3.3 μM per peptide), as a T-cell response was induced by both after two rounds of stimulation.

Fsp12 and fsp14

In some embodiments, the immunogenic fragment of mutSLC22A9 (SEQ ID NOs: 14-16) has an amino acid substitution compared to the naturally-occurring amino acid sequence of mutSLC22A9 (SEQ ID NOs: 14-16). In some embodiments, the immunogenic fragment is a fragment of mutSLC22A9(pos327) (SEQ ID NO: 14) or mutSLC22A9(pos335) (SEQ ID NO: 15), and has an amino acid substitution compared to the naturally-occurring amino acid sequence of the −1a frameshift mutant protein. In some embodiments, the immunogenic fragment is a fragment of SEQ ID NO: 17 or SEQ ID NO: 18, and comprises position 354 of SEQ ID NO: 17 or position 344 of SEQ ID NO: 18, respectively. In some embodiments, the immunogenic fragment comprises at least 8 consecutive amino acids from one of SEQ ID NOs: 17 and 18, including at position 354 of SEQ ID NO: 17 or position 344 of SEQ ID NO: 18, respectively. In some embodiments, the immunogenic fragment comprises at least 9 consecutive amino acids from one of SEQ ID NOs: 17 and 18, including at position 354 of SEQ ID NO: 17 or position 344 of SEQ ID NO: 18, respectively. In particular, position 354 of SEQ ID NO: 17 corresponds to position 354 of mutSLC22A9(pos327) (SEQ ID NO: 14), and position 344 of SEQ ID NO: 18 corresponds to position 344 of mutSLC22A((pos335) (SEQ ID NO: 15), which are both cysteine residues. The amino acid substitutions at position 354 of SEQ ID NO: 17 and position 344 of SEQ ID NO: 18 are from cysteine to any other amino acid. Thus, the amino acid at position 354 of SEQ ID NO: 17 and position 344 of SEQ ID NO: 18 is, independently, one of alanine, arginine, asparagine, aspartic acid, glutamine, glutamic acid, glycine, histidine, isoleucine, leucine, lysine, methionine, phenylalanine, proline, serine, threonine, tryptophan, tyrosine and valine. Preferably, the amino acid substitution is to glycine. Wherever there is reference below to the immunogenic fragment of mutSLC22A9 (SEQ ID NOs: 14-16) having an amino acid substitution from cysteine to any other amino acid, it is to be understood that the preferred amino acid substitution is from cysteine to glycine.

The advantage of this amino acid substitution, from cysteine, to any other amino acid, and particularly glycine, is the same as explained below in relation to the peptides capable of inducing an immune response against mutTGFβR2 (SEQ ID NO: 2).

In some embodiments, the immunogenic fragment of mutSLC22A9 (SEQ ID NOs: 14-16) having an amino acid substitution comprises no more than 28 amino acids.

In some embodiments, the immunogenic fragment of mutSLC22A9 (SEQ ID NOs: 14-16) having an amino acid substitution comprises position 348 to position 355, position 349 to position 356, position 350 to position 357, position 351 to position 358, position 352 to position 359, position 353 to position 360 or position 354 to position 361 of SEQ ID NO: 17, or position 338 to position 345, position 339 to position 346, position 340 to position 347, position 341 to position 348, position 342 to position 349, position 343 to position 350 or position 344 to position 351 of SEQ ID NO: 18.

In some embodiments, the immunogenic fragment of mutSLC22A9 (SEQ ID NOs: 14-16) having an amino acid substitution comprises position 348 to position 357, position 349 to position 358, position 350 to position 359, position 351 to position 360, position 352 to position 361, position 353 to position 362 or position 354 to position 363 of SEQ ID NO: 17, or position 338 to position 347, position 339 to position 348, position 340 to position 349, position 341 to position 350, position 342 to position 351, position 343 to position 352 or position 344 to position 353 of SEQ ID NO: 18

In some embodiments, the immunogenic fragment of mutSLC22A9 (SEQ ID NOs: 14-16) having an amino acid substitution comprises position 348 to position 362, position 349 to position 363, position 350 to position 364, position 351 to position 365, position 352 to position 366, position 353 to position 367 or position 354 to position 368 of SEQ ID NO: 17, or position 338 to position 352, position 339 to position 353, position 340 to position 354, position 341 to position 355, position 342 to position 356, position 343 to position 357 or position 344 to position 358 of SEQ ID NO: 18.

In some embodiments, the immunogenic fragment does not contain any amino acids from the wild-type amino acid sequence of SLC22A9 (SEQ ID NO: 13).

In some embodiments, the immunogenic fragment of mutSLC22A9 (SEQ ID NOs: 14-16) having an amino acid substitution comprises position 1 to position 8, position 1 to position 10, position 1 to position 15, position 2 to position 9, position 2 to position 11 or position 2 to position 16 of SEQ ID NO: 30. In some embodiments, the immunogenic fragment of mutSLC22A9 (SEQ ID NOs: 14-16) having an amino acid substitution comprises position 1 to position 8, position 1 to position 10 or position 1 to position 15 of SEQ ID NO: 30, and the immunogenic fragment is the N-terminus of the peptide. In other embodiments, the immunogenic fragment of mutSLC22A9 (SEQ ID NOs: 14-16) having an amino acid substitution comprises position 2 to position 9, position 2 to position 11 or position 2 to position 16 of SEQ ID NO: 30, and the immunogenic fragment starts at position two from the N-terminus of the peptide. In some embodiments, the immunogenic fragment of mutSLC22A9 (SEQ ID NOs: 14-16) having an amino acid substitution comprises the amino acid sequence of SEQ ID NO: 30. In some embodiments, the immunogenic fragment of mutSLC22A9 (SEQ ID NOs: 14-16) having an amino acid substitution consists of the amino acid sequence of SEQ ID NO: 30. In some embodiments, the peptide capable of inducing an immune response against mutSLC229 (SEQ ID NO: 14-16) consists of the amino acid sequence of SEQ ID NO: 30. When the peptide consists of the sequence of SEQ ID NO: 30, is referred to herein as “fsp12”.

In some embodiments, the immunogenic fragment of mutSLC22A9 (SEQ ID NOs: 14-16) having an amino acid substitution comprises position 1 to position 8, position 1 to position 10, position 1 to position 15, position 2 to position 9, position 2 to position 11, position 2 to position 16, position 3 to position 10, position 3 to position 12 or position 3 to position 17 of SEQ ID NO: 32. In some embodiments, the immunogenic fragment of mutSLC22A9 (SEQ ID NOs: 14-16) having an amino acid substitution comprises position 1 to position 8, position 1 to position 10 position 1 to position 15 of SEQ ID NO: 32, and the immunogenic fragment is the N-terminus of the peptide. In some embodiments, the immunogenic fragment of mutSLC22A9 (SEQ ID NOs: 14-16) having an amino acid substitution comprises position 2 to position 9, position 2 to position 11 or position 2 to position 16 of SEQ ID NO: 32, and the immunogenic fragment starts at position two from the N-terminus of the peptide. In some embodiments, the immunogenic fragment of mutSLC22A9 (SEQ ID NOs: 14-16) having an amino acid substitution comprises position 3 to position 12 or position 3 to position 17 of SEQ ID NO: 32, and the immunogenic fragment starts at position three from the N-terminus of the peptide. In some embodiments, the immunogenic fragment of mutSLC22A9 (SEQ ID NOs: 14-16) having an amino acid substitution comprises the amino acid sequence of SEQ ID NO: 32. In some embodiments, the immunogenic fragment of mutSLC22A9 (SEQ ID NOs: 14-16) having an amino acid substitution consists of the amino acid sequence of SEQ ID NO: 32. In some embodiments, the peptide capable of inducing an immune response against mutSLC229 (SEQ ID NO: 14-16) consists of the amino acid sequence of SEQ ID NO: 32. When the peptide consists of the sequence of SEQ ID NO: 32, is referred to herein as “fsp14”.

Peptide Mixture

The invention also provides mixtures comprising at least two different peptides selected from the above-described peptides and a peptide capable of inducing an immune response against a TGFβR2-1a frameshift mutant protein (mutTGFβR2; SEQ ID NO: 2) as described below. The peptide mixture comprises a first and a second peptide, wherein each of the first and the second peptide is, independently, a peptide capable of inducing an immune response against a TGFβR2-1a frameshift mutant protein (mutTGFβR2; SEQ ID NO: 2), a ASTE1-1a frameshift mutant protein (mutASTE1; SEQ ID NO: 5), a TAF1β-1a frameshift mutant protein (mutTAFβ1b; SEQ ID NO: 7), a KIAA2018-1a frameshift mutant protein (mutKIAA2018; SEQ ID NOs: 9-12) or a SLC22A9-1a frameshift mutant protein SEQ ID NOs: 14-16), as described herein, wherein the first peptide is different from the second peptide. Preferably, the first peptide is capable of inducing an immune response against a different frameshift mutant protein from the second peptide.

TGFβR2 Peptides

In some embodiments, in the peptide capable of inducing an immune response against a TGFβR2-1a frameshift mutant protein (mutTGFβR2; SEQ ID NO: 2), the immunogenic fragment comprises at least 8 consecutive amino acids of SEQ ID NO: 3, including at least one of positions 121 and 135, or at least 8 consecutive amino acids of SEQ ID NO: 19. In some embodiments, the immunogenic fragment comprises at least 9 consecutive amino acids. In some embodiments, the immunogenic fragment comprises at least 10 consecutive amino acids. In some embodiments, the immunogenic fragment comprises at least 12 consecutive amino acids. In some embodiments, the immunogenic fragment comprises at least 15 consecutive amino acids. In some embodiments, the immunogenic fragment comprises at least 17 consecutive amino acids. In some embodiments, the immunogenic fragment comprises at least 20 consecutive amino acids. In other embodiments, the immunogenic fragment comprises at least 24 consecutive amino acids. In further embodiments, the fragment comprises at least 33 consecutive amino acids.

In some embodiments, the immunogenic fragment comprises no more than 50 amino acids. In some embodiments, the immunogenic fragment comprises no more than 33 amino acids. In other embodiments, the immunogenic fragment comprises no more than 27, 24, 20, 17 or 9 amino acids.

In some embodiments, the peptide capable of inducing an immune response against mutTGFβR2 (SEQ ID NO: 2) comprises at least 9 amino acids. In some embodiments, the peptide comprises at least 17 amino acids. In some embodiments, the peptide comprises at least 20 amino acids. In other embodiments, the peptide comprises at least 24 amino acids. In some embodiments, the peptide comprises a least 27 amino acids. In further embodiments, the peptide comprises at least 33 amino acids.

In some embodiments, the peptide comprises no more than 33 amino acids. In some embodiments, the peptide comprises no more than 27 amino acids. In other embodiments, the peptide comprises no more than 24 amino acids. In some embodiments, the peptide comprises no more than 20 amino acids. In some embodiments, the peptide comprises no more than 17 amino acids. In further embodiments, the peptide comprises no more than 9 amino acids.

In some embodiments, the immunogenic fragment or the peptide comprises a glycine residue at the C-terminus thereof.

Bridging Region

In some embodiments, the immunogenic fragment of mutTGFβR2 (SEQ ID NO: 2) or peptide comprises at least one amino acid from the wild-type sequence of TGFβR2 (SEQ ID NO: 1) (i.e. position 1 to position 127 of SEQ ID NO: 2) consecutive with at least one amino acid from the amino acid sequence resulting from the frameshift mutation (i.e. position 128 to position 161 of SEQ ID NO: 2). Thus, the immunogenic fragment or peptide may be a fragment of mutTGFβR2 (SEQ ID NO: 2) which overlaps the amino acid sequence unaffected by the frameshift mutation and the amino acid sequence resulting from the frameshift mutation. In some embodiments, the immunogenic fragment or peptide comprises 2, 3, 4, 5, 6, 7 or 8 consecutive amino acids from the wild-type amino acid sequence of TGFβR2 (SEQ ID NO: 1). In some embodiments, the at least one amino acid from the wild-type sequence of TGFβR2 (SEQ ID NO: 1) is consecutive with 1, 2, 3, 4, 5, 6, 7, 8, 9, 10, 11, 12, 13, 14, 15, 16, 17, 18, 19, 20, 21, 22, 23, 24 or 25 amino acids from the amino acid sequence resulting from the frameshift mutation. In some embodiments, the immunogenic fragment or peptide has only 8 amino acids from the wild-type amino acid sequence of TGFβR2 (SEQ ID NO: 1). In some embodiments, the immunogenic fragment or peptide contains no amino acids from the wild-type amino acid sequence of TGFβR2 (SEQ ID NO: 1).

In some embodiments, the immunogenic fragment of mutTGFβR2 (SEQ ID NO: 2) comprises position 127 and position 128 of SEQ ID NO: 2, wherein the amino acid at position 127 of SEQ ID NO: 2 corresponds to the amino acid at position 127 of wild-type TGFβR2 (SEQ ID NO: 1). This corresponds to positions 8 and 9 of SEQ ID NO: 19, and positions 127 and 128 of SEQ ID NO: 3. In some embodiments, the immunogenic fragment comprises position 126 to position 128, position 125 to position 128, position 124 to position 128, position 123 to position 128, position 122 to position 128, position 121 to position 128 or position 120 to position 128 of SEQ ID NO: 2 or SEQ ID NO: 3 (which correspond to position 7 to position 9, position 6 to position 9, position 5 to position 9, position 4 to position 9, position 3 to position 9, position 2 to position 9, or position 1 to position 9 of SEQ ID NO: 19, respectively).

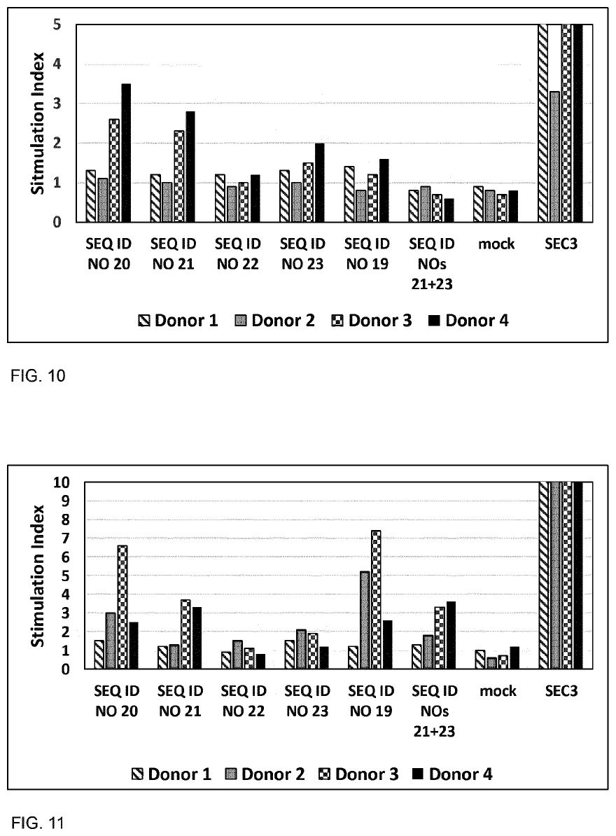

In particular, it is expected that at least the two amino acid residues bridging the wild-type amino acid sequence of TGFβR2 (SEQ ID NO: 1) and the amino acid sequence resulting from the frameshift mutation are helpful for providing an effective epitope, and for the peptide to have particularly good immunogenicity. In particular, FIGS. 10 and 11 show that peptides comprising amino acids from the wild-type sequence of TGFβR2 (SEQ ID NO: 1) (i.e. fsp1 (SEQ ID NO: 20), fsp2 (SEQ ID NO: 21) and fsp5 (SEQ ID NO: 19)) are more immunogenic than peptides which do not comprise amino acids from the wild-type sequence of TGFβR2 (SEQ ID NO: 1) (i.e. fsp3 (SEQ ID NO: 22) and fsp4 (SEQ ID NO: 23)). It is also expected that the presence of more than one amino acid from the wild-type sequence of TGFβR2 (SEQ ID NO: 1), consecutive with at least one amino acid from the amino acid sequence resulting from the frameshift mutation, is likely to improve the immunogenicity of the peptide. In particular, it is expected that five, six, seven or eight amino acids from the wild-type sequence of TGFβR2 (SEQ ID NO: 1), consecutive with at least one amino acid from the amino acid sequence resulting from the frameshift mutation, will be particularly helpful to the immunogenicity of the peptide. However, it is preferred that the peptide contains no more than eight amino acids from the wild-type sequence of TGFβR2, in order to balance improved immunogenicity with the requirement to reduce the risk of the peptide inducing an autoimmune response.

Fsp1, fsp3, fsp5 and fsp6

In some embodiments, the immunogenic fragment of mutTGFβR2 (SEQ ID NO: 2) comprises positions 6 to 13 of SEQ ID NO: 19, positions 10 to 18 of SEQ ID NO: 19, or positions 18 to 33 of SEQ ID NO: 19. In this instance, the peptide comprises no more than 40 amino acids. In some embodiments, the peptide comprises no more than 27 amino acids. In some embodiments, the immunogenic fragment comprises positions 6 to 17, positions 7 to position 22 or positions 16 to position 33 of SEQ ID NO: 19. In some embodiments, the immunogenic fragment comprises positions 2 to 22 of SEQ ID NO: 19. In some embodiments, the immunogenic fragment comprises positions 6 to 17 of SEQ ID NO: 19, and the immunogenic fragment starts at position six from the N-terminus of the peptide. In some embodiments, the immunogenic fragment comprises positions 2 to position 22 of SEQ ID NO: 19, and the immunogenic fragment starts at position two from the N-terminus of the peptide. In other embodiments, the immunogenic fragment comprises position 1 to position 17, position 1 to position 24, or position 1 to position 27, of SEQ ID NO: 19, and the immunogenic fragment is the N-terminus of the peptide. In some embodiments, the immunogenic fragment comprises position 16 to position 33 of SEQ ID NO: 19, and the immunogenic fragment starts at position three from the N-terminus of the peptide. In some embodiments, the immunogenic fragment comprises position 14 to position 33 of SEQ ID NO: 19. In some embodiments, the immunogenic fragment comprises position 14 to position 33 of SEQ ID NO: 19, and the fragment is the N-terminus of the peptide.

In some embodiments, the peptide comprises a glycine residue that the C-terminus thereof.

In some embodiments, the immunogenic fragment comprises the amino acid sequence of SEQ ID NO: 19, SEQ ID NO: 20, SEQ ID NO: 22, SEQ ID NO: 24 or SEQ ID NO 129. In some embodiments, the immunogenic fragment consists of the amino acid sequence of SEQ ID NO: 19, SEQ ID NO: 20, SEQ ID NO: 22, SEQ ID NO: 24 or SEQ ID NO: 129. In some embodiments, the peptide capable of inducing an immune response against mutTGFβR2 consists of the amino acid sequence of SEQ ID NO: 19, SEQ ID NO: 20, SEQ ID NO: 22, SEQ ID NO: 24 or SEQ ID NO: 129. When the peptide consists of the amino acid sequence of SEQ ID NO: 20, the peptide is referred to herein as “fsp1”. When the peptide consists of the amino acid sequence of SEQ ID NO: 22, the peptide is referred to herein as “fsp3”. When the peptide consists of the amino acid sequence of SEQ ID NO: 19, the peptide is referred to herein as “fsp5”. When the peptide consists of the amino acid sequence of SEQ ID NO: 24, the peptide is referred to herein as fsp6. When the peptide consists of the amino acid sequence of SEQ ID NO: 129, the peptide is referred to herein as “fsp17a”.

Fsp2, fsp4 and fsp6a

In some embodiments, the immunogenic fragment of mutTGFβR2 (SEQ ID NO: 2) has an amino acid substitution compared to the naturally-occurring amino acid sequence of mutTGFβR2 (SEQ ID NO: 2). Thus, the immunogenic fragment of mutTGFβR2 (SEQ ID NO: 2) may comprise at least 8 consecutive amino acids of SEQ ID NO: 3, and includes at least one of positions 121 and 135 of SEQ ID NO: 3. In some embodiments, the peptide comprises only one of positions 121 and 135 of SEQ ID NO: 3. In particular, positions 121 and 135 of SEQ ID NO: 3 correspond to positions 121 and 135 of mutTGFβR2 (SEQ ID NO: 2), respectively, which are both cysteine residues. The amino acid substitutions at positions 121 and 135 of SEQ ID NO: 3 are from cysteine to any other amino acid. Thus, the amino acid at positions 121 and 135 of SEQ ID NO: 3 is, independently, one of alanine, arginine, asparagine, aspartic acid, glutamine, glutamic acid, glycine, histidine, isoleucine, leucine, lysine, methionine, phenylalanine, proline, serine, threonine, tryptophan, tyrosine and valine. Preferably, the amino acid substitution is to glycine.

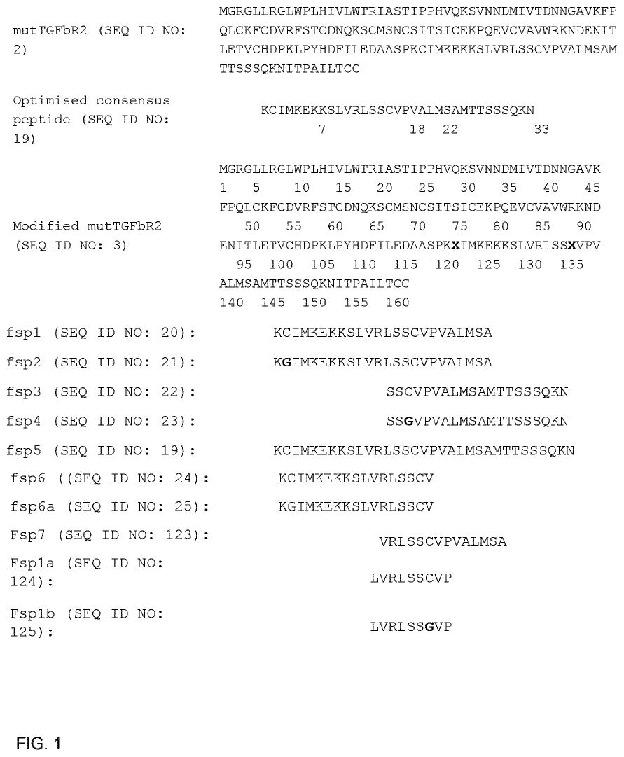

The amino acid substitution at position 121 and/or 135 of SEQ ID NO: 3, from cysteine to any other amino acid, is useful in preventing issues with production, stability, quality and immunology of the peptide. In particular, the peptides capable of inducing an immune response against TGFβR2 are derived from an optimised consensus sequence (SEQ ID NO: 19), as discussed in the Examples and shown in FIG. 1, and it has been found that the optimised consensus sequence can be difficult to synthesise due to its length. In addition, the solubility, stability and immunogenicity of the optimised consensus sequence (SEQ ID NO: 19) can be inadequate. In particular, the presence of one or more cysteine residues in a peptide can lead to molecular rearrangement and/or polymerisation of the peptide, due to the formation of inter- and/or intra-molecular disulphide bonds. This rearrangement and/or polymerisation may reduce the immunological potency of the peptide, and may induce unwanted inflammatory side effects through, for example, antibody formation and allergic reactions. The substitution of one or more cysteine residues in the peptide reduces the risk of these potential problems. Thus, in some embodiments, the substitution of one or more cysteine residues of the optimised consensus sequence improves the ease of production, the stability, the quality and the immunology of the peptide, but such substitutions are not essential for the present invention. FIG. 1 shows how the peptides having the amino acid substitutions relate to the optimised consensus sequence.

Range of Positions for Fsp2, Fsp4 and fsp6a

In some embodiments, the immunogenic fragment of mutTGFβR2 (SEQ ID NO: 2) having an amino acid substitution comprises position 115 to position 122, position 116 to position 123, position 117 to position 124, position 118 to position 125, position 119 to position 126, position 120 to position 127, position 121 to position 128, position 128 to position 135, position 129 to position 136, position 130 to position 137, position 131 to position 138, position 132 to position 139, position 133 to position 140, position 134 to position 141 or position 135 to position 142 of SEQ ID NO: 3.

In some embodiments, the immunogenic fragment of mutTGFβR2 (SEQ ID NO: 2) having an amino acid substitution comprises position 129 to position 137 of SEQ ID NO: 3.

In some embodiments, the immunogenic fragment of mutTGFβR2 (SEQ ID NO: 2) having an amino acid substitution comprises position 115 to position 126, position 116 to position 127, position 117 to position 128, position 118 to position 129, position 119 to position 130, position 120 to position 131, position 121 to position 132, position 124 to position 135, position 125 to position 136, position 126 to position 137, position 127 to position 138, position 128 to position 139, position 129 to position 140, position 130 to position 141, position 131 to position 142, position 132 to position 143, position 133 to position 144, position 134 to position 145 or position 135 to position 146 of SEQ ID NO: 3. In some embodiments, the immunogenic fragment of SEQ ID NO: 3 having an amino acid substitution comprises position 115 to position 129, position 116 to position 130, position 117 to position 131, position 118 to position 132, position 119 to position 133, position 120 to position 134, position 121 to position 135, position 122 to position 136, position 123 to position 137, position 124 to position 138, position 125 to position 139, position 126 to position 140, position 127 to position 141, position 128 to position 142, position 129 to position 143, position 130 to position 144, position 131 to position 145, position 132 to position 146, position 133 to position 147, position 134 to position 148 or position 135 to position 149 of SEQ ID NO: 3. In some embodiments, the immunogenic fragment of SEQ ID NO: 3 comprises position 119 to position 130, position 119 to position 133, position 120 to position 131, position 120 to position 134, position 121 to position 132, position 121 to position 135, position 133 to position 144, position 133 to position 147, position 134 to position 145, position 134 to position 148, position 135 to position 146, or position 135 to position 149 of SEQ ID NO: 3.

In some embodiments, the immunogenic fragment of mutTGFβR2 (SEQ ID NO: 2) having an amino acid substitution consists of position 115 to position 122, position 116 to position 123, position 117 to position 124, position 118 to position 125, position 119 to position 126, position 120 to position 127, position 121 to position 128, position 128 to position 135, position 129 to position 136, position 130 to position 137, position 131 to position 138, position 132 to position 139, position 133 to position 140, position 134 to position 141 or position 135 to position 142 of SEQ ID NO: 3

In some embodiments, the immunogenic fragment of mutTGFβR2 (SEQ ID NO: 2) having an amino acid substitution consists of position 129 to position 137 of SEQ ID NO: 3.

In some embodiments, the immunogenic fragment of mutTGFβR2 (SEQ ID NO: 2) having an amino acid substitution consists of position 115 to position 126, position 115 to position 129, position 116 to position 127, position 116 to position 130, position 117 to position 128, position 117 to position 131, position 118 to position 129, position 118 to position 132, position 119 to position 130, position 119 to position 133, position 120 to position 131, position 120 to position 134, position 121 to position 132, position 121 to position 135, position 122 to position 136, position 123 to position 137, position 124 to position 135, position 124 to position 138, position 125 to position 136, position 125 to position 139, position 126 to position 137, position 126 to position 140, position 127 to position 138, position 127 to position 141, position 128 to position 139, position 128 to position 142, position 129 to position 140, position 129 to position 143, position 130 to position 141, position 130 to position 144, position 131 to position 142, position 131 to position 145, position 132 to position 143, position 132 to position 146, position 133 to position 144, position 133 to position 147, position 134 to position 145, position 134 to position 148, position 135 to position 146 or position 135 to position 149 of SEQ ID NO: 3.

Fsp2, fsp6a (and Fsp5 with a G-to-C Substitution) and Fsp17

In some embodiments, the immunogenic fragment of mutTGFβR2 (SEQ ID NO: 2) having an amino acid substitution comprises position 121, but not position 135, of SEQ ID NO: 3. In some embodiments, the peptide comprises at least 70%, at least 75%, at least 80%, at least 85%, at least 90%, at least 95%, or has 100%, sequence identity to SEQ ID NO: 2 outside of the immunogenic fragment. In some such embodiments, the peptide comprises 100% sequence identity to SEQ ID NO: 2 outside of the fragment. In some embodiments, the peptide comprises no more than 33 amino acids, no more than 27 amino acids, no more than 24 amino acids or no more than 17 amino acids. In some embodiments, the peptide consists of 33 amino acids. In some embodiments, the peptide consists of 27 amino acids. In other embodiments, the peptide consists of 24 amino acids. In other embodiments, the peptide consists of 17 amino acids. In some embodiments, the immunogenic fragment of SEQ ID NO: 3 comprises position 119 to position 126, position 120 to position 127 or position 121 to position 128 of SEQ ID NO: 3. In some embodiments, the immunogenic fragment of SEQ ID NO: 3 comprises position 119 to position 130, position 120 to position 131, position 121 to position 132 of SEQ ID NO: 3. In other embodiments, the fragment comprises position 119 to position 133, position 120 to position 134, position 121 to position 135 of SEQ ID NO: 3. In some embodiments, the immunogenic fragment or the peptide has a glycine residue at the C-terminus thereof.

In some embodiments, the immunogenic fragment of mutTGFβR2 (SEQ ID NO: 2) having an amino acid substitution consists of position 119 to position 126, position 120 to position 127 or position 121 to position 128 of SEQ ID NO: 3. In some embodiments, the immunogenic fragment consists of position 119 to position 130, position 119 to position 133, position 120 to position 131, position 120 to position 134, position 121 to position 132 or position 121 to position 135 of SEQ ID NO: 3. In some embodiments, the immunogenic fragment starts at position one, two or three from the N-terminus of the peptide. In some embodiments, the immunogenic fragment is the N-terminus of the peptide. In some embodiments, position 121 of SEQ ID NO: 3 is glycine.

In some embodiments, the peptide comprises an immunogenic fragment of mutTGFβR2 (SEQ ID NO: 2) having an amino acid substitution wherein the immunogenic fragment consists of position 119 to position 126, position 119 to position 130, position 119 to position 133, position 120 to position 127, position 120 to position 131, position 120 to position 134, position 121 to position 128, position 121 to position 132, position 121 to position 135 of SEQ ID NO: 3, wherein position 121 of SEQ ID NO: 3 is glycine, and wherein the peptide has at least 94% sequence identity to SEQ ID NO: 2 outside of the immunogenic fragment and comprises no more than 33 amino acids. In some embodiments, the peptide comprises an immunogenic fragment of mutTGFβR2 (SEQ ID NO: 2) having an amino acid substitution wherein the immunogenic fragment consists of position 119 to position 126, position 119 to position 130, position 119 to position 133, position 120 to position 127, position 120 to position 131, position 120 to position 134, position 121 to position 128, position 121 to position 132, position 121 to position 135 of SEQ ID NO: 3, wherein position 121 of SEQ ID NO: 3 is glycine, and wherein the peptide has 100% sequence identity to SEQ ID NO: 2 outside of the immunogenic fragment and comprises no more than 33 amino acids. In some embodiments, the immunogenic fragment is the N-terminus of the peptide. In some such embodiments, the peptide consists of 33 amino acids. In some embodiments, the peptide consists of 27 amino acids, and the peptide may consist of the amino acid sequence of SEQ ID NO: 128. Peptides consisting of the amino acid sequence of SEQ ID NO: 128 are referred to herein as “fsp17”. In other embodiments, the peptide consists of 24 amino acids, and the peptide may consist of the amino acid sequence of SEQ ID NO: 21. Peptides consisting of the amino acid sequence of SEQ ID NO: 21 are referred to herein as “fsp2”. In other embodiments, the peptide consists of 17 amino acids, and the peptide may consist of the amino acid sequence SEQ ID NO: 25. Peptides consisting of the amino acid sequence of SEQ ID NO: 25 are referred to herein as “fsp6a”.

In some embodiments, the peptide comprises an immunogenic fragment consisting of SEQ ID NO: 25 and the peptide comprises one or more additional amino acids at the C-terminus of the fragment. For example, the peptide may comprise one, two, three, four, five, six, seven, eight, nine or 10 additional amino acids at the C-terminus of the fragment. In some embodiments, the immunogenic fragment consisting of SEQ ID NO: 25 is the N-terminus of the peptide. In some embodiments, the fragment consisting of SEQ ID NO: 25 is the N-terminus of the peptide and the peptide comprises seven additional amino acids at the C-terminus of the fragment. In some embodiments, the fragment consisting of SEQ ID NO: 25 is the N-terminus of the peptide and the peptide comprises 10 additional amino acids at the C-terminus of the fragment. In other embodiments, the fragment consisting of SEQ ID NO: 25 is the N-terminus of the peptide and the peptide consists of seven additional amino acids at the C-terminus of the fragment. In some embodiments, the fragment consisting of SEQ ID NO: 25 is the N-terminus of the peptide and the peptide consists of 10 additional amino acids at the C-terminus of the fragment. In some embodiments, the one or more additional amino acids have at least 70%, at least 75%, at least 80%, at least 85%, at least 90%, at least 95%, or has 100%, sequence identity to the corresponding amino acids of SEQ ID NO: 2, and, preferably, have at least 95% or 100% sequence identity to the corresponding amino acids of SEQ ID NO: 2.

Fsp4

In some embodiments, the immunogenic fragment of mutTGFβR2 (SEQ ID NO: 2) having an amino acid substitution comprises position 135, but not position 121, of SEQ ID NO: 3. In some embodiments, the peptide comprises at least 70%, at least 75%, at least 80%, at least 85%, at least 90%, at least 95%, or has 100%, sequence identity to SEQ ID NO: 2 outside of the immunogenic fragment. In some embodiments, the peptide comprises no more than 20 amino acids, while in other embodiments the peptide consists of 20 amino acids. In some embodiments, the immunogenic fragment of mutTGFβR2 (SEQ ID NO: 2) having an amino acid substitution comprises position 133 to position 144, position 134 to position 145 or position 135 to position 146 of SEQ ID NO: 3. In some embodiments, the immunogenic fragment of mutTGFβR2 (SEQ ID NO: 2) having an amino acid substitution comprises position 133 to position 147, position 134 to position 148 or position 135 to position 149 of SEQ ID NO: 3. In some embodiments, the immunogenic fragment of mutTGFβR2 (SEQ ID NO: 2) having an amino acid substitution consists of position 133 to position 144, position 133 to position 147, position 134 to position 145, position 134 to position 148, position 135 to position 146 or position 135 to position 149 of SEQ ID NO: 3. In some embodiments, the immunogenic fragment of mutTGFβR2 (SEQ ID NO: 2) having an amino acid substitution starts at position one, two or three from the N-terminus of the peptide. In some embodiments, the immunogenic fragment of mutTGFβR2 (SEQ ID NO: 2) having an amino acid substitution is the N-terminus of the peptide. In some embodiments, position 135 of SEQ ID NO: 3 is glycine. In some embodiments, the immunogenic fragment or the peptide has a glycine residue at the C-terminus thereof.

In some embodiments, the peptide comprises an immunogenic fragment of mutTGFβR2 (SEQ ID NO: 2) having an amino acid substitution wherein the fragment consists of position 133 to position 144, position 133 to position 147, position 134 to position 145, position 134 to position 148, position 135 to position 146 or position 135 to position 149 of SEQ ID NO: 3, wherein position 135 of SEQ ID NO: 3 is glycine, and wherein the peptide has 100% sequence identity to SEQ ID NO: 2 outside of the fragment and comprises no more than 20 amino acids. In some embodiments, the immunogenic fragment is the N-terminus of the peptide. In some embodiments, the peptide consists of 20 amino acids. In some embodiments, the peptide consists of the amino acid sequence of SEQ ID NO: 23, and such peptides are referred to herein as “fsp4”.

Fsp1a & fsp1b

In some embodiments, the immunogenic fragment of mutTGFβR2 (SEQ ID NO: 2) comprises at least 8 consecutive amino acids of SEQ ID NO: 19 or comprises at least 8 consecutive amino acids of SEQ ID NO: 3 including at least position 135 of SEQ ID NO: 3. In some embodiments, the immunogenic fragment comprises at least 9 consecutive amino acids of SEQ ID NO: 19 or comprises at least 9 consecutive amino acids of SEQ ID NO: 3 including position 135 of SEQ ID NO: 3. In some embodiments, peptide comprises at least 70%, at least 75%, at least 80%, at least 85%, at least 90%, at least 95%, or has 100%, sequence identity to SEQ ID NO: 2 outside of the immunogenic fragment. In some embodiments, the immunogenic fragment or peptide has a glycine residue at the C-terminal thereof.

In some embodiments, the peptide capable of inducing an immune response against mutTGFβR2 (SEQ ID NO: 2) comprises at least 8 consecutive amino acids of SEQ ID NO: 19 or comprises at least 8 consecutive amino acids of SEQ ID NO: 3 including at least position 135 of SEQ ID NO: 3. In some embodiments, the peptide comprises 9 consecutive amino acids of SEQ ID NO: 19 or comprises 9 consecutive amino acids of SEQ ID NO: 3 including position 135 of SEQ ID NO: 3. In some embodiments, the immunogenic fragment consists of 9 consecutive amino acids of SEQ ID NO: 19 or consists of at least 9 consecutive amino acids of SEQ ID NO: 3 including position 135 of SEQ ID NO: 3.

In some embodiments, the immunogenic fragment of mutTGFβR2 (SEQ ID NO: 2) comprises positions 10 to 17 or positions 11 to 18 of SEQ ID NO: 19. In some embodiments, the immunogenic fragment comprises positions 10 to 18 of SEQ ID NO: 19. In some embodiments, the immunogenic fragment comprises the amino acid sequence of SEQ ID NO: 124. In some embodiments, the immunogenic fragment consists positions 10 to 18 of SEQ ID NO: 19, and has the amino acid sequence of SEQ ID NO: 124. In some embodiments, the peptide consists of the amino acid sequence of SEQ ID NO: 124, and such peptides are referred to herein as “fsp1a”.

In some embodiments, the immunogenic fragment of mutTGFβR2 (SEQ ID NO: 2) comprises at least 8 amino acids of SEQ ID NO: 3 including position 135 of SEQ ID NO: 3, and, therefore, comprises an amino acid substitution compared with the naturally-occurring amino acid sequence of mutTGFβR2 (SEQ ID NO: 2). In some embodiments, the immunogenic fragment of mutTGFβR2 (SEQ ID NO: 2) comprises positions 129 to 136 or positions 130 to 137 of SEQ ID NO: 3. In some embodiments, the immunogenic fragment comprises positions 129 to 137 of SEQ ID NO: 3. In some embodiments, the amino acid at position 135 of SEQ ID NO: 3 is glycine. In some embodiments, the immunogenic fragment comprises the amino acid sequence of SEQ ID NO: 125. In some embodiments, the peptide consists of the amino acid sequence of SEQ ID NO: 125, and such peptides are referred to herein as “fsp1b”.

As explained in Example 5, it is predicted that the amino acid sequence of SEQ ID NO: 124 or SEQ ID NO: 125 is immunogenic in view of the difference in immunogenicity of fsp6 (SEQ ID NO: 24) and fsp7 (SEQ ID NO: 123), and the similarities and differences between these amino acid sequences.

Peptide Mixtures

The peptide mixtures of the invention can contain any number and any combination of the peptides disclosed herein, so long as the peptide mixtures comprise a first and a second peptide which induce immune responses against different −1a frameshift mutant proteins.

In some embodiments, the first peptide is a peptide capable of inducing an immune response to against a TGFβR2-1a frameshift mutant protein (SEQ ID NO: 2), and the second peptide is a peptide capable of inducing an immune response against a ASTE1-1a frameshift mutant protein (SEQ ID NO: 5), a TAF1β-1a frameshift mutant protein (SEQ ID NO: 7), a KIAA2018-1a frameshift mutant protein (SEQ ID NOs: 9-12) or a SLC22A9-1a frameshift mutant protein (SEQ ID NOs: 14-16). In some embodiments, the first peptide is a peptide capable of inducing an immune response to against a TGFβR2-1a frameshift mutant protein (SEQ ID NO: 2), and the second peptide is a peptide capable of inducing an immune response against a ASTE1-1a frameshift mutant protein (SEQ ID NO: 5) or a TAF1β-1a frameshift mutant protein (SEQ ID NO: 7). In some embodiments, the first peptide is a peptide capable of inducing an immune response against a ASTE1-1a frameshift mutant protein (SEQ ID NO: 5) and the second peptide is a peptide capable of inducing an immune response against a TGFβR2-1a frameshift mutant protein (SEQ ID NO: 2) or a TAF1β-1a frameshift mutant protein (SEQ ID NO: 7). In particular, FIGS. 14,15 and 22 show that a peptide mixture comprising peptides derived from mutTGFβR2, mutASTE1 and mutTAF1β is capable of inducing a T-cell response. FIGS. 16-18 show that the individual peptides within the peptide mixture are immunogenic, as the individual peptide are capable of inducing an immune response after even only one round of stimulation with a peptide mixture comprising these peptides. FIG. 21 also shows that an individual peptide of mutASTE1 is capable of inducing an immune response after even only one round of stimulation with a peptide mixture comprising this peptide.

In some embodiments, the first peptide comprises the amino acid sequence of one of SEQ ID NOs: 19-25, 124, 125 or 126 and the second peptide comprises the amino acid sequence of SEQ ID NO: 26, SEQ ID NO: 27, SEQ ID NO: 127 or SEQ ID NO: 128. In these embodiments, the first peptide may comprise no more than 33, 27, 24, 20, 17 or 9 amino acids, and the second peptide may comprise no more than 31, 30 or 25 amino acids. In some embodiments, the first peptide consists of the amino acid sequence of one of SEQ ID NOs: 19-25, 124, 125 and 126, and the second peptide consists of the amino acid sequence of SEQ ID NO: 26, SEQ ID NO: 27, SEQ ID NO: 127 or SEQ ID NO: 128.

In some embodiments, the peptide mixture comprises at least one further peptide selected from a peptide capable of inducing an immune response against a TGFβR2-1a frameshift mutant protein (mutTGFβR2; SEQ ID NO: 2), a ASTE1-1a frameshift mutant protein (mutASTE1; SEQ ID NO: 5), a TAF1β-1a frameshift mutant protein (mutTAFβ1b; SEQ ID NO: 7), a KIAA2018-1a frameshift mutant protein (mutKIAA2018; SEQ ID NOs: 9-12) or a SLC22A9-1a frameshift mutant protein SEQ ID NOs: 14-16), as described herein, wherein the at least one further peptide is different from each of the first and second peptide. In some embodiments, the at least one further peptide is capable of inducing an immune response against a different frameshift mutant protein from each of the first and second peptides.

The peptide mixture comprising at least one further peptide may be any of the peptide mixtures set out in Table 1 below, wherein the peptides are as described herein. Thus, all of the peptides described herein can be combined according to Table 1 to form the peptide mixtures of the invention.

TABLE 1

| First | Second | | | |
|---|---|---|---|---|
| peptide | peptide | Third peptide | Fourth peptide | Fifth peptide |
| TGFβR2 | ASTE1 | TGFβR2, ASTE1, | n/a | n/a |
| | | TAF1β, KIAA2018 or | | |
| | | SLC22A9 | | |
| TGFβR2 | TAF1β | TGFβR2, TAF1β, | n/a | n/a |
| | | ASTE1, KIAA2018 or | | |
| | | SLC22A9 | | |
| TGFβR2 | KIAA2018 | TGFβR2, KIAA2018, | n/a | n/a |
| | | ASTE1, TAF1β or | | |
| | | SLC22A9 | | |
| TGFβR2 | SLC22A9 | TGFβR2, SLC22A9, | n/a | n/a |
| | | ASTE1, TAF1β or | | |
| | | KIAA2018 | | |
| ASTE1 | KIAA2018 | ASTE1, KIAA2018, | n/a | n/a |
| | | TGFβR2, TAF1β or | | |
| | | SLC22A9 | | |
| ASTE1 | SLC22A9 | ASTE1, SLC22A9, | n/a | n/a |
| | | TGFβR2, TAF1β or | | |
| | | KIAA2018 | | |
| TAF1β | ASTE1 | TAF1β, ASTE1, | n/a | n/a |
| | | TGFβR2, KIAA2018 | | |
| | | or SLC22A9 | | |
| TAF1β | KIAA2018 | TAF1β, KIAA2018, | n/a | n/a |
| | | TGFβR2, ASTE1 or | | |
| | | SLC22A9 | | |
| TAF1β | SLC22A9 | TAF1β, SLC22A9, | n/a | n/a |
| | | TGFβR2, ASTE1 or | | |
| | | KIAA2018 | | |
| KIAA2018 | SLC22A9 | KIAA2018, | n/a | n/a |
| | | SLC22A9, TGFβR2, | | |
| | | TAF1β or ASTE1 | | |
| TGFβR2 | ASTE1 | TAF1β | KIAA2018, | n/a |
| | | | TGFβR2, | |
| | | | ASTE1 or | |
| | | | TAF1β | |
| TGFβR2 | ASTE1 | TAF1β | SLC22A9, | |
| | | | TGFβR2, | |
| | | | ASTE1 or | |
| | | | TAF1β | |
| TGFβR2 | ASTE1 | KIAA2018 | SLC22A9, | n/a |
| | | | TGFβR2, | |
| | | | ASTE1 or | |
| | | | KIAA2018 | |
| ASTE1 | TAF1β | KIAA2018 | SLC22A9, | n/a |
| | | | ASTE1, TAF1β | |
| | | | or KIAA2018 | |
| TGFβR2 | ASTE1 | TAF1β | KIAA2018 | SLC22A9, |
| | | | | TGFβR2, |
| | | | | ASTE1, TAF1β |
| | | | | or KIAA2018 |

Where a peptide mixture as shown in Table 1 comprises more than one peptide derived from the same protein, these peptides are different from one another and can be derived from the same −1a frameshift mutant protein or, where there is more than one −1a frameshift mutant of the protein, the peptides can be derived from different −1a frameshift mutants of the same protein.

In some embodiments, the first peptide is a peptide capable of inducing an immune response to against a TGFβR2-1a frameshift mutant protein (SEQ ID NO: 2), the second peptide is a peptide capable of inducing an immune response against a ASTE1-1a frameshift mutant protein (SEQ ID NO: 5), and the at least one further peptide is a peptide capable of inducing an immune response against a TAF1β-1a frameshift mutant protein (mutTAFβ1b; SEQ ID NO: 7), a KIAA2018-1a frameshift mutant protein (mutKIAA2018; SEQ ID NOs: 9-12) or a SLC22A9-1a frameshift mutant protein SEQ ID NOs: 14-16). In some embodiments, the first peptide is a peptide capable of inducing an immune response to against a TGFβR2-1a frameshift mutant protein (SEQ ID NO: 2), the second peptide is a peptide capable of inducing an immune response against a TAF1β-1a frameshift mutant protein (mutTAFβ1b; SEQ ID NO: 7), and the at least one further peptide is a peptide capable of inducing an immune response against a ASTE1-1a frameshift mutant protein (SEQ ID NO: 5), a KIAA2018-1a frameshift mutant protein (mutKIAA2018; SEQ ID NOs: 9-12) or a SLC22A9-1a frameshift mutant protein SEQ ID NOs: 14-16). In some embodiments, the first peptide is a peptide capable of inducing an immune response to against a TGFβR2-1a frameshift mutant protein (SEQ ID NO: 2), the second peptide is a peptide capable of inducing an immune response against a ASTE1-1a frameshift mutant protein (SEQ ID NO: 5), and the at least one further peptide is a peptide capable of inducing an immune response against a TAF1β-1a frameshift mutant protein (mutTAFβ1b; SEQ ID NO: 7). FIGS. 14-18 show that a peptide mixture containing these three peptides induces an immune response, and that each of the peptides in the mixture is immunogenic after even only one round of stimulation with the peptide mixture.

In some embodiments, the first peptide is a peptide capable of inducing an immune response against a TGFβR2-1a frameshift mutant protein, wherein the peptide comprises i) an immunogenic fragment of SEQ ID NO: 3, wherein the fragment comprises at least 8 consecutive amino acids of SEQ ID NO: 3 including at least one of positions 121 and 135 of SEQ ID NO: 3, or ii) an immunogenic fragment of SEQ ID NO: 2, wherein the fragment comprises at least 8 consecutive amino acids of SEQ ID NO: 19, the second peptide is a peptide capable of inducing an immune response against a ASTE1-1a frameshift mutant protein, wherein the peptide comprises an immunogenic fragment of SEQ ID NO: 5, wherein the fragment comprises at least 8 consecutive amino acids of SEQ ID NO: 26, and the at least one further peptide is a peptide capable of inducing an immune response against a TAF1β-1a frameshift mutant protein, wherein the peptide comprises an immunogenic fragment of SEQ ID NO: 7, wherein the fragment comprises at least 8 consecutive amino acids of SEQ ID NO: 27.

In some embodiments, the peptide mixture comprises a first, second, third and fourth peptide. In some such embodiments, the first peptide is a peptide capable of inducing an immune response against a TGFβR2-1a frameshift mutant protein, wherein the peptide comprises i) an immunogenic fragment of SEQ ID NO: 3, wherein the fragment comprises at least 8 consecutive amino acids of SEQ ID NO: 3 including at least one of positions 121 and 135 of SEQ ID NO: 3, or ii) an immunogenic fragment of SEQ ID NO: 2, wherein the fragment comprises at least 8 consecutive amino acids of SEQ ID NO: 19, the second peptide is a peptide capable of inducing an immune response against a ASTE1-1a frameshift mutant protein, wherein the peptide comprises an immunogenic fragment of SEQ ID NO: 5, wherein the fragment comprises at least 8 consecutive amino acids of SEQ ID NO: 26, and the third peptide is a peptide capable of inducing an immune response against a TAF1β-1a frameshift mutant protein, wherein the peptide comprises an immunogenic fragment of SEQ ID NO: 7, wherein the fragment comprises at least 8 consecutive amino acids of SEQ ID NO: 27, and the fourth peptide is a peptide capable of inducing an immune response against a KIAA2018-1a frameshift mutant protein, wherein the peptide comprises a immunogenic fragment of one of SEQ ID NOs: 9-12, wherein the fragment comprises at least 8 consecutive amino acids of one of one of SEQ ID NOs: 9-12, respectively, including at least one amino acid from positions 13 to 37 of SEQ ID NO: 9, positions 91 to 109 of SEQ ID NO: 10, positions 147 to 167 of SEQ ID NO: 11 and positions 1016 to 1037 of SEQ ID NO: 12, respectively, or a peptide capable of inducing an immune response against a SLC22A9-1a frameshift mutant protein, wherein the peptide comprises an immunogenic fragment of one of SEQ ID NO: 14-18, wherein the fragment comprises at least 8 consecutive amino acids of one of SEQ ID NOs: 14-18, respectively, including at least one amino acid from positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18, respectively.

In some embodiments, the peptide mixture comprises a first, second, third, fourth and fifth peptide. In some such embodiments, the first peptide is a peptide capable of inducing an immune response against a TGFβR2-1a frameshift mutant protein, wherein the peptide comprises i) an immunogenic fragment of SEQ ID NO: 3, wherein the fragment comprises at least 8 consecutive amino acids of SEQ ID NO: 3 including at least one of positions 121 and 135 of SEQ ID NO: 3, or ii) an immunogenic fragment of SEQ ID NO: 2, wherein the fragment comprises at least 8 consecutive amino acids of SEQ ID NO: 19, the second peptide is a peptide capable of inducing an immune response against a ASTE1-1a frameshift mutant protein, wherein the peptide comprises an immunogenic fragment of SEQ ID NO: 5, wherein the fragment comprises at least 8 consecutive amino acids of SEQ ID NO: 26, and the third peptide is a peptide capable of inducing an immune response against a TAF1β-1a frameshift mutant protein, wherein the peptide comprises an immunogenic fragment of SEQ ID NO: 7, wherein the fragment comprises at least 8 consecutive amino acids of SEQ ID NO: 27, and the fourth peptide is a peptide capable of inducing an immune response against a KIAA2018-1a frameshift mutant protein, wherein the peptide comprises a immunogenic fragment of one of SEQ ID NOs: 9-12, wherein the fragment comprises at least 8 consecutive amino acids of one of one of SEQ ID NOs: 9-12, respectively, including at least one amino acid from positions 13 to 37 of SEQ ID NO: 9, positions 91 to 109 of SEQ ID NO: 10, positions 147 to 167 of SEQ ID NO: 11 and positions 1016 to 1037 of SEQ ID NO: 12, respectively, and the fifth peptide is a peptide capable of inducing an immune response against a SLC22A9-1a frameshift mutant protein, wherein the peptide comprises an immunogenic fragment of one of SEQ ID NO: 14-18, wherein the fragment comprises at least 8 consecutive amino acids of one of SEQ ID NOs: 14-18, respectively, including at least one amino acid from positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18, respectively.

In some embodiments, the first peptide comprises of the amino acid sequence of one of SEQ ID NOs: 19-25, 124, 125 and 126, and the second peptide comprises the amino acid sequence of SEQ ID NO: 26 or SEQ ID NO: 127, and the at least one further peptide comprises the amino acid sequence of SEQ ID NO: 27 or SEQ ID NO: 128. In these embodiments, the first peptide may comprise no more than 33, 27, 24, 20, 17 or 9 amino acids, the second peptide may comprise no more than 31 or 25 amino acids and the at least one further peptide may comprise no more than 30 or 25 amino acids. In some embodiments, the first peptide consists of the amino acid sequence of one of SEQ ID NOs: 19-25, 124, 125 and 126, the second peptide consists of the amino acid sequence of SEQ ID NO: 26 or SEQ ID NO: 127, and the at least one further peptide consists of the amino acid sequence of SEQ ID NO: 27 or SEQ ID NO: 128.

In some embodiments, the first peptide consists of the amino acid sequence of SEQ ID NO: 21, the second peptide consists of the amino acid sequence of SEQ ID NO: 26 and the at least one further peptide consists of the amino acid sequence of SEQ ID NO: 27. In some embodiments, the peptide mixture consists of these three peptides.

In some embodiments, the first peptide consists of the amino acid sequence of SEQ ID NO: 126, the second peptide consists of the amino acid sequence of SEQ ID NO: 127 and the at least one further peptide consists of the amino acid sequence of SEQ ID NO: 128. In some embodiments, the peptide mixture consists of these three peptides. In some embodiments, the at least one further peptide is a different peptide from each of the first and second peptides but induces an immune response against the same −1a frameshift mutant protein as one of the first and second peptides. Thus, for example, one of the first or second peptide may be a peptide which is capable of inducing an immune response against mutTGFβR2 (SEQ ID NO: 2), and the at least one further peptide may also be a peptide which is capable of inducing an immune response against mutTGFβR2 (SEQ ID NO: 2) and which is a different peptide from the first or second peptide which is a peptide which is capable of inducing an immune response against mutTGFβR2 (SEQ ID NO: 2). In another example, one of the first and second peptides may be a peptide which is capable of inducing an immune response against mutSLC22A9 (SEQ ID NOs: 14-16), and the at least one further peptide may also be a peptide which induces an immune response against mutSLC22A9 (SEQ ID NOs: 14-16) but which is a different peptide from the first or second peptide which is capable of inducing an immune response against mutSLC22A9 (SEQ ID NOs: 14-16). Similarly, when the peptide mixture comprises more than one further peptide, the at least one further peptide may comprise at least two peptides which are different from each other but which are capable of inducing an immune response against the same −1a frameshift mutant protein.

In some embodiments, one of the first and second peptide, and the at least one further peptide, induce an immune response against different −1a frameshift mutants of the same protein. Thus, for example, the first or second peptide may be a peptide which induces an immune response against mutKIAA2018(pos13) (SEQ ID NO: 9) and the at least one further peptide may be a peptide which induces an immune response against mutKIAA2018(pos91) (SEQ ID NO: 10), mutKIAA2018(pos147) (SEQ ID NO: 11) or mutKIAA2018(pos1016) (SEQ ID NO: 12). In another example, the first or second peptide may be a peptide which induces an immune response against mutSLC22A9(pos327) (SEQ ID NO: 14) and the at least one further peptide may be a peptide which induces an immune response against mutSLC22A9(pos335) (SEQ ID NO: 15) or mutSLC22A9(pos553) (SEQ ID NO: 16). Similarly, when the peptide mixture comprises more than one further peptide, the at least one further peptide may comprise at least two peptides which are capable of inducing immune responses against different −1a frameshift mutants of the same protein.

In some embodiments, the first peptide comprises the amino acid sequence of SEQ ID NO: 28, the second peptide comprises the amino acid sequence of SEQ ID NO: 29 and the at least one further peptide comprises the amino acid sequence of SEQ ID NO: 31. In some embodiments, the first peptide consists of the amino acid sequence of SEQ ID NO: 28, the second peptide consists of the amino acid sequence of SEQ ID NO: 29 and the at least one further peptide consists of the amino acid sequence of SEQ ID NO: 31. In some embodiments, the peptide mixture consists of these three peptides.

In some embodiments, the peptide mixture comprises at least 2, 3, 4, 5, 6, 7, 8, 9, 10, 11, 12, 13, 14, 15 or 16 different peptides. In some embodiments, the peptide mixture comprises no more than 2, 3, 4, 5, 6, 7, 8, 9, 10, 11, 12, 13, 14, 15 or 16 different peptides. In some embodiments, the peptide mixture comprises 3 different peptides. In some embodiments, the peptide mixture comprises 5 different peptides. In some embodiments, the peptide mixture comprises 10 different peptides. In some embodiments, the peptide mixture comprises no more than 10 different peptides. In some embodiments, the peptide mixture consists of 3 different peptides. In some embodiments, the peptide mixture consists of 5 different peptides. In other embodiments, the peptide mixture consists of 10 different peptides.

The peptide mixtures may contain the peptides in equal or different proportions. In some embodiments, the first and second peptides are present in the mixture in equal proportions, i.e. each peptide comprises 50% of the peptide component of the peptide mixture. In other embodiments, there is a greater proportion of the first peptide in the peptide mixture than the second peptide. For example, the first peptide may comprise at least 55%, at least 60%, at least 70%, at least 80% or at least 90% of the peptide component of the peptide mixture. In alternative embodiments, there is a greater proportion of the second peptide in the peptide mixture than the first peptide. For example, the second peptide may comprise at least 55%, at least 60%, at least 70%, at least 80% or at least 90% of the peptide component of the peptide mixture. In embodiments comprising at least one further peptide, the peptides are present in the peptide component of the peptide mixture in equal proportions. In other embodiments, the first, second and the at least one further peptide are present in different proportions from each other. For example, each of the first, second and at least one further peptide may independently comprise at least 1%, at least 5%, at least 10%, at least 20% at least 30%, at least 40%, at least 50%, at least 60%, at least 60%, at least 70%, at least 80% or at least 90% of the peptide component of the peptide mixture.

Nucleic Acids

In another aspect of the present invention, there is provided at least one nucleic acid molecule or molecules which individually or collectively comprise nucleotide sequences encoding at least two of the peptides in the peptide mixtures of the disclosures above, or the or each nucleic acid encodes a nucleotide sequence encoding at least one of the peptides of the disclosures above. Thus, in some embodiments, each nucleic acid molecule encodes one or more of the peptides of the disclosures above, or encodes two or more of the peptides in the peptide mixture of the disclosures above. In some embodiments, each nucleic acid molecule encodes only one of the peptides of the disclosures above. In some embodiments, each nucleic acid molecule encodes 2, 3, 4, 5, 6 or 7 peptides disclosed above. In some embodiments, each nucleic acid molecule encodes 2, 3, 4, 5, 6, 7, 8, 9, 10, 11, 12, 13, 14, 15 or 16 peptides of the peptide mixture disclosed above.

There is also provided a mixture of nucleic acid molecules, wherein each nucleic acid molecule of the mixture comprises a nucleotide sequence which encodes a different peptide of a peptide mixture according to the disclosures above. Thus, the mixture of nucleic acid molecules collectively encodes the peptide mixture of the disclosures above.

In some embodiments, the nucleic acid molecules and mixtures of nucleic acid molecules are used to synthesise the peptides and peptides mixtures of the disclosures above. For example, one or more peptides described above, or one or more peptides of the peptide mixtures described above, may be synthesised by administering one or more nucleic acid molecule to a subject, whereupon each nucleic acid molecule is expressed by the subject, thereby giving rise to one or more peptide in situ. The peptide(s) produced then elicits an immune response in the subject. In another example, the nucleic acid molecule(s) may be used to synthesise one or more peptide described above, or one or more peptide of the peptide mixtures described above, in vitro, by transforming or transfecting a host cell with the nucleic acid molecule, such that the host cell expresses the nucleic acid molecule to produce the peptide. The peptide is then recovered and purified. In some embodiments, the peptides described above, and the peptides of the peptide mixtures described above, are produced by chemical synthesis, using methods well known in the art.

T-Cell Receptors

In another aspect of the invention, there is provided a T-cell receptor, or an antigen-binding fragment thereof, specific for a peptide according to the disclosures above, when presented on an MHC molecule.

In some embodiments, the T-cell receptor is an αβ T-cell receptor, or the antigen-binding fragment of the T-cell receptor is an antigen-binding fragment of an αβ T-cell receptor. In these embodiments, the T-cell receptor, or antigen-binding fragment thereof, is specific for a peptide according to the disclosures herein when presented on an MHC molecule.

In some embodiments, the T-cell receptor is a γδ T-cell receptor, or the antigen-binding fragment of the T-cell receptor is an antigen-binding fragment of a γδ T-cell receptor. In these embodiments, the T-cell receptor does not necessarily require presentation of the peptide on an MHC molecule in order to recognise the peptide.

T-Cells and T-Cell Mixtures

In another aspect of the present invention, there is provided a T-cell, and a T-cell preparation comprising one or more T-cells, specific for a peptide according to the disclosures above.

There is further provided a T-cell mixture comprising T-cells specific for each of the peptides in one of the peptide mixtures of the disclosures above. When a T-cell in the T-cell mixture is specific for a mutTGFβR2 peptide, the T-cell is a non-transfected T-cell. Thus, the T-cell mixture comprises first and second T-cells specific for first and second peptides, respectively, wherein the first and second peptides are independently selected from a mutTGFβR2 peptide according to the disclosures above, a mutASTE1 peptide according to the disclosures above, a mutTAF1β peptide according to the disclosures above, a mutKIAA2018 peptide according to the disclosures above and a mutSLC22A9 peptide according to the disclosures above, wherein, when a T-cell is specific for a mutTGFβR2 peptide, the T-cell is a non-transfected T-cell, and wherein the first peptide is capable of inducing an immune response against a different frameshift mutant protein from the second peptide

The T-cell, T-cell preparation and T-cell mixture may be ex vivo and may be produced by stimulating, ex vivo, at least one reactive T-cell with a peptide or a peptide mixture according to the disclosures above. For example, in one embodiment, the T-cell is specific for a peptide capable of inducing an immune response against a ASTE1-1a frameshift mutant protein, wherein the peptide comprises an immunogenic fragment of SEQ ID NO: 5, wherein the fragment comprises at least 10 consecutive amino acids of SEQ ID NO: 26. In another embodiment, the T-cell is specific for a peptide capable of inducing an immune response against a TAF1β-1a frameshift mutant protein, wherein the peptide comprises an immunogenic fragment of SEQ ID NO: 7, wherein the fragment comprises at least 10 consecutive amino acids of SEQ ID NO: 27. In another embodiment, the T-cell is specific for a peptide capable of inducing an immune response against a KIAA2018-1a frameshift mutant protein, wherein the peptide comprises a immunogenic fragment of one of SEQ ID NOs: 9-12, wherein the fragment comprises at least 8 consecutive amino acids of one of SEQ ID NOS: 9-12, including at least one amino acid from positions 13 to 37 of SEQ ID NO: 9, positions 91 to 109 of SEQ ID NO: 10, positions 147 to 167 of SEQ ID NO: 11 and positions 1016 to 1037 of SEQ ID NO: 12, respectively. In other embodiments, the T-cell is specific for a peptide capable of inducing an immune response against a SLC22A9-1a frameshift mutant protein, wherein the peptide comprises an immunogenic fragment of one of SEQ ID NO: 14-18, wherein the fragment comprises at least 8 consecutive amino acids of one of SEQ ID NOs: 14-18, including at least one amino acid from positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18, respectively.

In another embodiment, the T-cell preparation comprises one or more T-cells specific for a peptide capable of inducing an immune response against a ASTE1-1a frameshift mutant protein, wherein the peptide comprises an immunogenic fragment of SEQ ID NO: 5, wherein the fragment comprises at least 10 consecutive amino acids of SEQ ID NO: 26; a TAF1β-1a frameshift mutant protein, wherein the peptide comprises an immunogenic fragment of SEQ ID NO: 7, wherein the fragment comprises at least 10 consecutive amino acids of SEQ ID NO: 27; a KIAA2018-1a frameshift mutant protein, wherein the peptide comprises a immunogenic fragment of one of SEQ ID NOs: 9-12, wherein the fragment comprises at least 8 consecutive amino acids of one of SEQ ID NOS: 9-12, including at least one amino acid from positions 13 to 37 of SEQ ID NO: 9, positions 91 to 109 of SEQ ID NO: 10, positions 147 to 167 of SEQ ID NO: 11 and positions 1016 to 1037 of SEQ ID NO: 12; or a SLC22A9-1a frameshift mutant protein, wherein the peptide comprises an immunogenic fragment of one of SEQ ID NO: 14-18, wherein the fragment comprises at least 8 consecutive amino acids of one of SEQ ID NOs: 14-18, including at least one amino acid from positions 327 to 400 of SEQ ID NO: 14, positions 335 to 400 of SEQ ID NO: 15, positions 533 to 549 of SEQ ID NO: 16, positions 327 to 400 of SEQ ID NO: 17 and positions 335 to 400 of SEQ ID NO: 18.

Where the T-cell receptor of any T-cell disclosed herein is an αβ T-cell receptor, then the T-cell receptor is specific for the peptide when presented on an MHC molecule. Where the T-cell receptor of any T-cell disclosed herein is a γδ T-cell receptor, then the T-cell receptor does not necessarily require presentation of the peptide on an MHC molecule in order to recognise the peptide.

Vector & Host Cell

In another aspect of the present invention, there is provided a vector comprising a nucleic acid molecule comprising a nucleotide sequence according to the disclosures above. The nucleotide sequence, therefore, encodes at least one of the peptides as disclosed above, or at least one of the peptides in the peptide mixtures as disclosed above. In some embodiments, the vector comprises a nucleic acid molecule comprising a nucleotide sequence which encodes all of the peptides of a peptide mixture according to the disclosure above.

In some embodiments, the vector is a DNA vector or a RNA vector.

In a further aspect, there is provided a host cell comprising a vector as described above. The host cell is transfected or transformed with the vector, such that the host cell expresses the nucleic acid molecule(s) encoded by the vector. The host cell may be any cell type that is capable of being transfected with a vector and expressing the vector. In some embodiments, the host cell is a plant cell, an animal cell, a micro-organism, or a yeast cell. In some embodiments, the host cell is a dendritic cell.

In some embodiments, the host cell may contain more than one vector, wherein each vector comprises a nucleic acid molecule comprising a nucleotide sequence encoding a different peptide as described above. Thus, the host cell may comprise multiple vectors, each encoding a different peptide, such that the host cell expresses more than one type of nucleic acid molecule and, therefore, more than one peptide.

Pharmaceutical Compositions

Pharmaceutical compositions comprising the peptides, peptide mixtures, T-cell receptors or antigen-binding fragments thereof, T-cells, T-cell mixtures, T-cell preparations, nucleic acid molecule(s), vectors or host cells described above are also provided. Such pharmaceutical compositions may also comprise at least one pharmaceutically acceptable carrier, diluent and/or excipient. For example, the pharmaceutically acceptable carrier, diluent and/or excipient may be saline or sterilised water. In some embodiments, the pharmaceutical composition further comprises one or more additional active ingredients and/or adjuvants. In certain embodiments, the pharmaceutical composition may further comprise one or more ingredients therapeutically effective for the same disease indication. In one embodiment, the pharmaceutical composition of the present invention may further comprise one or more further chemotherapeutic agents, one or more cancer vaccines, one or more antibodies, one or more small molecules and/or one or more immune stimulants (for example, cytokines). In some embodiments, the peptide, peptide mixture, T-cell receptor or antigen-binding fragment thereof, T-cell, T-cell preparation, T-cell mixture, nucleic acid molecule(s), vector, host cell or the pharmaceutical composition may be used in combination with other forms of immunotherapy, including other cancer vaccines. In some embodiments, the peptide, peptide mixture, T-cell receptor or antigen-binding fragment thereof, T-cell, T-cell preparation, T-cell mixture, nucleic acid, vector, host cell or the pharmaceutical composition is used in combination with one or more cancer vaccines derived from a different cancer antigen.

Use

Peptides, peptide mixtures, T-cell receptors, T-cells, T-cell preparations, T-cell mixtures, nucleic acid molecules, vectors, host cells and pharmaceutical composition disclosed above are for use in the treatment and/or prophylaxis of cancer, and in particular cancers associated with a frameshift mutation, preferably a −1a frameshift mutation, in one or more of TGFβR2, ASTE1, TAF1β, KIAA2018 and SLC22A9. In particular, it is common for cancer patients who have a frameshift mutation to have more than one frameshift mutations, as the underlying cause of these cancers is failure of DNA mismatch repair (MMR). Preferably, the treatment and/or prophylaxis of cancer is in humans. In particular, about 15% of all CRCs, and about 44% of all MSI-H cancers, have a frameshift mutation in TGFβR2. In addition, TAF1β, ASTE1 and TGFβR2 are three of the four most frequently mutated genes in MSI CRCs, and a frameshift mutation in each of these genes is independently found in 75% of MSI-H CRCs. In addition, a frameshift mutation in KIAA2018, SLC22A9 and ASTE1 is found about 51%, 50% and 45%, respectively, of all MSI-H cancers. The cancer may be colorectal cancer or stomach cancer. The colorectal cancer may be colon cancer or rectal cancer. The peptides, peptide mixtures, T-cell receptors, T-cells, T-cell preparations, T-cell mixtures, nucleic acid molecules, vectors and host cells may be used for the treatment and/or prophylaxis of more than one of these types of cancer. In particular, the peptides, peptide mixtures, T-cell receptors, T-cells, T-cell preparations, T-cell mixtures, nucleic acid molecules, vectors and host cells of the disclosures above can be used to treat all MSI colorectal cancers and a large proportion of all MSI-H cancers. Thus, the present invention provides an effective treatment for a large proportion of cancers, particularly colorectal cancer and stomach cancer, and more particularly, hereditary colorectal cancer.

In some embodiments, a peptide capable of inducing an immune response against a ASTE1-1a frameshift mutant protein, wherein the peptide comprises an immunogenic fragment of SEQ ID NO: 5, wherein the fragment comprises at least 10 consecutive amino acids of SEQ ID NO: 26, is particularly useful as a vaccine or treatment against cancer. In some embodiments, the peptide comprises no more than 31 amino acids, and the peptide has at least 95% sequence identity to SEQ ID NO: 5 outside of the immunogenic fragment. In some embodiments, the peptide comprises no more than 25 amino acids, and the peptide has 100% sequence identity to SEQ ID NO: 5 outside of the immunogenic fragment. In some embodiments, the peptide consists of 25 amino acids, and the immunogenic fragment consists of 12 or 15 consecutive amino acids of SEQ ID NO: 26. In some embodiments, the peptide consists of 31 amino acids, and the immunogenic fragment consists of 12 or 15 consecutive amino acids of SEQ ID NO: 26. In some embodiments, the peptide has only 3 amino acids, or has no amino acids, from the wild-type amino acid sequence of ASTE1 (SEQ ID NO: 4). In some embodiments, the peptide has a glycine residue at the C-terminus thereof. In some embodiments, the peptide consists of the amino acid sequence of SEQ ID NO: 26 or SEQ ID NO: 127. In particular, it has been found that the peptide consisting of the amino acid sequence of SEQ ID NO: 26 (i.e. fsp8) is particularly useful as a vaccine or treatment against cancer because it is able to induce an immune response, as shown in FIGS. 16-18, 21 and 22. In particular, any increase in T-cell proliferation above the control (i.e. T-cell+APC) is indicative of a T-cell response to the peptide. FIGS. 16-18 show the T-cell proliferative response induced by each of SEQ ID NO: 21 (fsp2), SEQ ID NO: 26 (fsp8) and SEQ ID NO: 27 (fsp9) after stimulation of donors with a peptide mixture containing these three peptides, and these three Figures show an increase in T-cell proliferation in response to induction by SEQ ID NO: 26 (fsp8). FIG. 21 shows the T-cell proliferative response induced by SEQ ID NO: 127 (fsp15) after stimulation of donors with a peptide mixture containing SEQ ID NO: 126 (fsp17), SEQ ID NO: 127 (fsp15) and SEQ ID NO: 128 (fsp16), and FIG. 21 shows an increase in T-cell proliferation in response to induction by SEQ ID NO: 127 (fsp15). FIG. 22 shows the T-cell proliferative response induced by SEQ ID NO: 127 (fsp15) individually, and a peptide mixture containing SEQ ID NO: 126 (fsp17), SEQ ID NO: 127 (fsp15) and SEQ ID NO: 128 (fsp16), after stimulation of donors with a peptide mixture containing SEQ ID NO: 126 (fsp17), SEQ ID NO: 127 (fsp15) and SEQ ID NO: 128 (fsp16), and FIG. 22 shows increased T-cell proliferation in response to induction by the peptide mixture and SEQ ID NO: 127 (fsp15).

In some embodiments, a peptide capable of inducing an immune response against a TAF1β-1a frameshift mutant protein, wherein the peptide comprises an immunogenic fragment of SEQ ID NO: 7, wherein the fragment comprises at least 10 consecutive amino acids of SEQ ID NO: 27, is particularly useful as a vaccine or treatment against cancer. In some embodiments, the peptide comprises no more than 25 amino acids, and has 100% sequence identity to SEQ ID NO: 7 outside of the immunogenic fragment. In some embodiments, the peptide consists of 25 amino acids, and the immunogenic fragment consists of 12 or 15 consecutive amino acids of SEQ ID NO: 27. In some embodiments, the peptide consists of the amino acid sequence of SEQ ID NO: 27. In particular, it has been found that the peptide consisting of the amino acid sequence of SEQ ID NO: 27 (i.e. fsp9) is particularly useful as a vaccine or treatment against cancer because it is able to induce an immune response, as shown in FIGS. 16-18 and 22. In particular, any increase in T-cell proliferation above the control (i.e. T-cell+APC) is indicative of a T-cell response to the peptide. FIGS. 16-18 show the T-cell proliferative response to each of SEQ ID NO: 21 (fsp2), SEQ ID NO: 26 (fsp8) and SEQ ID NO: 27 (fsp9) after stimulation of donors with a peptide mixture containing these three peptides, and these three Figures show an increase in T-cell proliferation in response to induction by SEQ ID NO: 27 (fsp9). FIG. 22 shows the T-cell proliferative response induced by a peptide mixture containing SEQ ID NO: 126 (fsp17), SEQ ID NO: 127 (fsp15) and SEQ ID NO: 128 (fsp16), after stimulation of donors with a peptide mixture containing SEQ ID NO: 126 (fsp17), SEQ ID NO: 127 (fsp15) and SEQ ID NO: 128 (fsp16), and FIG. 22 shows increased T-cell proliferation in response to induction by the peptide mixture.

In some embodiments, a peptide mixture comprising a peptide capable of inducing an immune response against a TGFβR2-1a frameshift mutant protein, a peptide capable of inducing an immune response against a ASTE1-1a frameshift mutant protein and a peptide capable of inducing an immune response against a TAF1β-1a frameshift mutant protein. In some embodiments, the peptide capable of inducing an immune response against a ASTE1-1a frameshift mutant protein comprises an immunogenic fragment of SEQ ID NO: 5, wherein the fragment comprises at least 8 consecutive amino acids of SEQ ID NO: 26. In some embodiments, the peptide comprises no more than 31 amino acids, and the peptide has at least 95% sequence identity to SEQ ID NO: 5 outside of the immunogenic fragment. In some embodiments, the peptide capable of inducing an immune response against a ASTE1-1a frameshift mutant protein comprises no more than 25 amino acids, and the peptide has 100% sequence identity to SEQ ID NO: 5 outside of the immunogenic fragment. In some embodiments, the peptide capable of inducing an immune response against a ASTE1-1a frameshift mutant protein consists of 25 amino acids, and the immunogenic fragment consists of 12 or 15 consecutive amino acids of SEQ ID NO: 26. In some embodiments, the peptide capable of inducing an immune response against a ASTE1-1a frameshift mutant protein consists of 31 amino acids, and the immunogenic fragment consists of 12 or 15 consecutive amino acids of SEQ ID NO: 26. In some embodiments, the peptide capable of inducing an immune response against a ASTE1-1a frameshift mutant protein has only 3 amino acids, or has no amino acids, from the wild-type amino acid sequence of ASTE1 (SEQ ID NO: 4). In some embodiments, the peptide capable of inducing an immune response against a ASTE1-1a frameshift mutant protein has a glycine residue at the C-terminus thereof. In some embodiments, the peptide capable of inducing an immune response against a ASTE1-1a frameshift mutant protein consists of the amino acid sequence of SEQ ID NO: 26 or SEQ ID NO: 127. In some embodiments, the peptide capable of inducing an immune response against a TAF1β-1a frameshift mutant protein comprises an immunogenic fragment of SEQ ID NO: 7, wherein the fragment comprises at least 8 consecutive amino acids of SEQ ID NO: 27. In some embodiments, the peptide capable of inducing an immune response against a −1a TAF1β frameshift mutant peptide comprises no more than 30 amino acids, and has at least 95% sequence identity to SEQ ID NO: 7 outside of the immunogenic fragment. In some embodiments, the peptide capable of inducing an immune response against a TAF1β-1a frameshift mutant protein comprises no more than 25 amino acids, and has 100% sequence identity to SEQ ID NO: 7 outside of the immunogenic fragment. In some embodiments, the peptide capable of inducing an immune response against a TAF1β-1a frameshift mutant protein consists of 25 amino acids, and the immunogenic fragment consists of 12 or 15 consecutive amino acids of SEQ ID NO: 27. In some embodiments, the peptide capable of inducing an immune response against a −1a TAF1β frameshift mutant peptide consists of 30 amino acids, and the immunogenic fragment consists of 12 or 15 consecutive amino acids of SEQ ID NO: 27. In some embodiments, the peptide capable of inducing an immune response against a −1a TAF1β frameshift mutant peptide has only 5 amino acid, or only 2 amino acids, from the wild-type amino acid sequence of TAF1β (SEQ ID NO: 6). In some embodiments, the peptide capable of inducing an immune response against a −1a TAF1β frameshift mutant peptide has a glycine residue at the C-terminus thereof. In some embodiments, the peptide capable of inducing an immune response against a TAF1β-1a frameshift mutant protein consists of the amino acid sequence of SEQ ID NO: 27 or SEQ ID NO: 128. In some embodiments, the peptide capable of inducing an immune response against a TGFβR2-1a frameshift mutant protein comprises an immunogenic fragment of SEQ ID NO: 3, wherein the fragment comprises at least 8 consecutive amino acids of SEQ ID NO: 3 including positions 121 of SEQ ID NO: 3. In some embodiments, the peptide capable of inducing an immune response against a TGFβR2-1a frameshift mutant protein comprises no more than 33 amino acids. In some embodiments, the peptide capable of inducing an immune response against a TGFβR2-1a frameshift mutant protein comprises at least 12 or 15 consecutive amino acids of SEQ ID NO: 3, including position 121, but not position 135, of SEQ ID NO: 3, wherein the amino acid at position 121 of SEQ ID NO: 3 is glycine, and the peptide has at least 90% sequence identity to SEQ ID NO: 2 outside of the immunogenic fragment. In some embodiments, the peptide capable of inducing an immune response against a TGFβR2-1a frameshift mutant protein comprises at least 12 or 15 consecutive amino acids of SEQ ID NO: 3, including position 121, but not position 135, of SEQ ID NO: 3, wherein the amino acid at position 121 of SEQ ID NO: 3 is glycine, and the peptide has 100% sequence identity to SEQ ID NO: 2 outside of the immunogenic fragment. In some embodiments the peptide capable of inducing an immune response against a TGFβR2-1a frameshift mutant protein consists of 17, 24, 27 or 33 amino acids, and the fragment of SEQ ID NO: 3 consists of 12 or 15 consecutive amino acids of SEQ ID NO: 3. In some embodiments, the peptide capable of inducing an immune response against a TGFβR2-1a frameshift mutant protein consists of the amino acid sequence of SEQ ID NO: 21 or SEQ ID NO: 126. In particular, FIGS. 14 and 15 show that that a peptide mixture containing a peptide consisting of the amino acid sequence of SEQ ID NO: 21 (i.e. fsp2), a peptide consisting of the amino acid sequence of SEQ ID NO: 26 (i.e. fsp8) and a peptide consisting of the amino acid sequence of SEQ ID NO: 27 (i.e. fsp9) induced T-cell proliferation after two or three rounds of stimulation with this peptide mixture. In addition, FIGS. 16-18 show that T-cells were induced by each of the individual peptides in this peptide mixture (comprising fsp2, fsp8 and fsp9) after one or two rounds of stimulation with the peptide mixture. Thus, the individual peptides are immunogenic even when administered as a peptide mixture. FIG. 22 shows that a peptide mixture containing a peptide consisting of the amino acid sequence of SEQ ID NO: 126 (i.e. fsp17), a peptide consisting of the amino acid sequence of SEQ ID NO: 127 (i.e. fsp15) and a peptide consisting of the amino acid sequence of SEQ ID NO: 128 (fsp16) induced T-cell proliferation after two rounds of stimulation with this peptide mixture. FIG. 22 also shows that the peptide consisting of SEQ ID NO: 126 (i.e. fsp17) induced T-cells after two rounds of stimulation with this peptide mixture.

In some embodiments, more than one peptide according to the disclosures above is administered to the subject. In some embodiments, each peptide is administered separately to the subject.

The T-cell, or the T-cells in the T-cell preparation or T-cell mixture, for use in the treatment and/or prophylaxis of cancer may be autologous or allogenic. For example, heterologous T-cells may be administered to a patient where the T-cells are from a donor having the same or similar HLA repertoire as the patient.

The peptide, peptide mixture, vector, host cell or pharmaceutical composition of the invention may be administered to a subject by any suitable delivery technique known to those skilled in the art. For example, among other techniques, the peptide, peptide mixture or pharmaceutical composition may be administered to a subject by injection, in the form of a solution, in the form of liposomes or in dry form (for example, in the form of coated particles, etc). The host cell may be administered, for example, by transfusion. The vector may be administered, for example, by injection subcutaneously or into the tumour. In some embodiments, the peptide, peptide mixture or pharmaceutical composition may be administered in an amount, for example, of between 1 μg and 1 g of each peptide once every three days, once a week, once a month, once every three months, once every four months or once every six months. In some embodiments, the net amount of each peptide per dose is 60 nM. For example, for intradermal injection, each peptide may be present in a volume of 0.1 ml at a concentration of 0.6 mM.

In some embodiments, the peptide or peptide mixture is administered with an adjuvant or immune stimulator, such as GM-CSF. In embodiments using GM-CSF, this may be any GM-CSF, for example, glycosylated GM-CSF or non-glycosylated GM-CSF. GM-CSF may be administered in an amount of between 0.5 and 120 μg/m², between 1 and 120 μg/m², between 2 and 115 μg/m², between 3 and 110 μg/m², between 4 and 105 μg/m², between 5 and 100 μg/m², between 6 and 95 μg/m², between 7 and 90 μg/m², between 48 and 85 μg/m², between 9 and 80 μg/m², between 10 and 75 μg/m², between 11 and 70 μg/m², between 12 and 65 μg/m², between 13 and 60 μg/m², between 14 and 55 μg/m², between 15 and 50 μg/m², between 16 and 45 μg/m², between 17 and 40 μg/m², or between 18 and 40 μg/m² of body surface area. In some embodiments, GM-CSF is administered at a dosage of between 1 μg and 200 μg, between 5 μg and 175 μg, between 5 μg and 150 μg, between 5 μg and 125 μg, between 5 μg and 100 μg, between 10 μg and 100 μg, between 20 μg and 90 μg, between 25 μg and 80 μg, between 25 μg and 70 μg, between 25 μg and 65 μg, or between 30 μg and 60 μg, per dose. In some embodiments, non-glycosylated GM-CSF is administered at a dosage of 30 μg per dose. In other embodiments, glycosylated GM-CSF is administered at a dosage of 60 μg dose. In embodiments where GM-CSF is administered by intradermal injection, the dose may be a 0.1 ml solution containing GM-CSF at a concentration of 0.3 mg/ml or 0.6 mg/ml. In some embodiments, the peptide, peptide mixture or pharmaceutical composition may be administered in an amount, for example, of between 1 μg and 1 g of each peptide once every three days, once a week, once a month, once every three months, once every four months or once every six months.

The T-cell receptors of the present invention may be transfected to T-cells of a patient having a HLA allele matching the HLA allele for which the T-cell receptor is specific using methods known in the art. In particular, T-cells are obtained from the patient, the T-cells are transfected with a vector encoding the T-cell receptors, and the transfected T-cells are re-introduced to the patient.

The T-cells, T-cell mixtures and T-cell preparations of the present invention may be administered by intra-venous injection and/or infusion, and may be administered in an amount, for example, of between 10⁶ and 10¹² of each T-cell specific for a peptide of the peptide mixture or peptide once every month, once every two months, once every three months, once every six months or once a year. Preferably, the dosage is administered once every month for between 2 and 5 months and is subsequently administered less frequently.

The nucleic acid and mixture of nucleic acids of the present invention may be administered by intra-muscular injection and/or subcutaneous injection.

Administration of a peptide or a peptide mixture of the present invention to a subject, or expression of the peptide or peptide mixture by a subject, elicits an immune response to the peptide or peptide mixture, in particular a T-cell mediated immune response. The peptide, or each peptide of the peptide mixture, may be processed by an antigen-presenting cell (APC) and may be presented on an MHC molecule. αβ T-cells are activated by binding of the T-cell receptor to a peptide presented on a MHC molecule by the APC, thereby resulting in an immune response against tumour cells having a mutation corresponding to that present in the administered peptide(s). γδ T-cells do not necessarily require antigen processing or presentation of the antigen by MHC molecules.

In another aspect of the invention, there is provided a peptide, peptide mixture, T-cell receptor, T-cell, T-cell preparation, T-cell mixture, nucleic acid molecule or a pharmaceutical composition for use in a method of comprising the diagnosis of cancer and the selection of an appropriate treatment. The method comprises the steps of (i) identifying whether a cancer patient is MSI-H and, if so, (ii) selecting a peptide, peptide mixture, T-cell receptor, T-cell, T-cell preparation, T-cell mixture, nucleic acid molecule or pharmaceutical composition according to the disclosures above. In some embodiments, the method further comprises, in step (i), testing whether the patient has a frameshift mutation in one or more of the TGFβR2, ASTE1, TAF1@, KIAA2018 and SLC22A9 protein, and, if so, selecting a peptide, peptide mixture, T-cell receptor, T-cell, T-cell preparation, T-cell mixture, nucleic acid molecule or pharmaceutical composition according to the disclosures above. In some embodiment, the frameshift mutation is a −1a frameshift mutation. In some embodiments, the method further comprises (iii) administering the selected peptide, peptide mixture, T-cell receptor, T-cell, T-cell preparation, T-cell mixture, nucleic acid molecule or pharmaceutical composition to the patient.

In another aspect of the present invention, there is provided a method of treating and/or preventing cancer comprising administering a peptide, peptide mixture, non-transfected T-cell, non-transfected T-cell preparation, non-transfected T-cell mixture, nucleic acid molecule or pharmaceutical composition according to the disclosures above to a patient in need thereof. The method may comprise the steps of (i) identifying a cancer patient as MSI-H, and (ii) administering a peptide, peptide mixture, non-transfected T-cell, non-transfected T-cell preparation, non-transfected T-cell mixture, nucleic acid molecule or pharmaceutical composition according to the disclosures above to the patient. The method may further comprise, in step (i), the step of identifying that the patient has a frameshift mutation in the TGFβR2 protein. In some embodiments, the frameshift mutation is a −1a frameshift mutation.

In another aspect of the invention, there is provided a kit that includes a peptide, a peptide mixture, a T-cell receptor, a T-cell, a T-cell mixture, a T-cell preparation, a nucleic acid molecule, a nucleic acid molecule mixture, a vector and/or a host cell according to the disclosures above. The peptide, peptide mixture, T-cell receptor, T-cell, T-cell mixture, T-cell preparation, nucleic acid, nucleic acid mixture, vector and/or host cell as such may be present in the kit, or the peptide, peptide mixture, T-cell receptor, T-cell, T-cell mixture, T-cell preparation, nucleic acid molecule, nucleic acid mixture, vector and/or host cell may be present as a pharmaceutical formulation. In some embodiments, the peptide, peptide mixture, T-cell receptor, T-cell, T-cell preparation, T-cell mixture, nucleic acid molecule mixture, vector and/or host cell may be packaged, for example in a vial, bottle, flask, which may be further packaged, for example, within a box, envelope or bag. In some embodiments, the kit comprises a peptide mixture, a T-cell mixture and/or nucleic acid molecule mixture wherein the peptides, the T-cells and/or the nucleic acid molecules are provided in separate containers, such that the peptides, T-cells and/or nucleic acid molecules are mixed by the user.

### EXAMPLES

### Example 1—TGFβR2 Peptides

Previous studies on peptides for use as a vaccine against cancers associated with TGFβR2 having a frameshift mutation showed that at least some peptides may be immunogenic, but there are contradictory results. Thus, a consensus peptide was manually predicted based upon the peptides tested in earlier studies.

TABLE 2
Previously tested peptides of mutTGFβR2 and their T-cell activation
result

| | T cell | Peptide |
|---|---|---|
| Previously Tested Peptide Sequence | activation | label |
| SPKCIMKEKKSLVRLSSCVPVA (SEQ ID NO: 35) | + | P540 |
| SLVRLSSCVPVALMSAMTTSSSQ (SEQ ID NO: 34) | + | p538 |
| SLVRLSSCV (SEQ ID NO: 37) | + | p523 |
| RLSSCVPVA (SEQ ID NO: 33) | + | p573 |
| ALMSAMTTSSSQKNITPAILTCC (SEQ ID NO: 38) | - | p539 |
| AMTTSSSQKNITPAILTCC (SEQ ID NO: 39) | - | p537 |

Manually predicted consensus sequence:

(SEQ ID NO: 40)
SPKCIMKEKKSLVRLSSCVPVALMSAMTTSSSQ.

An online algorithm (i.e. SYFPEITHI) was then used to predict epitopes of mutTGFβR2 for HLA class II alleles. The HLA class II alleles included in the search were: HLA-DRB1*0101, HLA-DRB1*0301 (DR17), HLADRB1*0401 (DR4Dw4), HLA-DRB1*0701, HLA-DRB1*1101, HLA-DRB1*1501 (DR2b).

A cut-off prediction score of 20 was used, wherein HLA binding strength increases with the prediction score. SYFPEITHI produced the following predicted epitopes:

TABLE 3
SYFPEITHI search results

| Peptide sequence | Prediction score (SYFPEITHI) |
|---|---|
| KKSLVRLSSCVPVAL (SEQ ID NO: 41) | 31 (HLA-DRB1*0101) |
| | 20 (HLA-DRB1*0401) |
| VALMSAMTTSSSQKN (SEQ ID NO: 42) | 28 (HLA-DRB1*0101) |
| | 20 (HLA-DRB1*0401) |
| PVALMSAMTTSSSQK (SEQ ID NO: 43) | 23 (HLA-DRB1*0101) |
| | 26 (HLA-DRB1*0401) |
| | 22 (HLA-DRB1*0701) |
| KCIMKEKKSLVRLSS (SEQ ID NO: 44) | 24 (HLA-DRB1*1501) |
| CVPVALMSAMTTSSS (SEQ ID NO: 45) | 23 (HLA-DRB1*0101) |
| | 24 (HLA-DRB1*1501) |
| LVRLSSCVPVALMSA (SEQ ID NO: 46) | 22 (HLA-DRB1*0101) |
| | 21 (HLA-DRB1*0301) |
| | 24 (HLA-DRB1*1501) |
| MKEKKSLVRLSSCVP (SEQ ID NO: 47) | 22 (HLA-DRB1*1101) |
| KSLVRLSSCVPVALM (SEQ ID NO: 48) | 20 (HLA-DRB1*0701) |
| | 20 (HLA-DRB1*1501) |
| SSCVPVALMSAMTTS (SEQ ID NO: 49) | 20 (HLA-DRB1*0401) |
| | 22 (HLA-DRB1*0701) |

As a result, SYFPEITHI predicted the consensus sequence:

(SEQ ID NO: 19)
KCIMKEKKSLVRLSSCVPVALMSAMTTSSSQKN.

The manually predicted consensus sequence (SEQ ID NO 40) and the SYFPEITHI predicted consensus sequence (SEQ ID NO: 19) were compared and an optimised consensus sequence was produced.

TABLE 4
Comparison of predicted consensus sequences

| | |
|---|---|
| Manually-predicted | SPKCIMKEKKSLVRLSSCVPVALMSAMT |
| consensus peptide | TSSSQ (SEQ ID NO: 40) |
| SYFPEITHI predicted | KCIMKEKKSLVRLSSCVPVALMSAMTTS |
| consensus peptide | SSQKN (SEQ ID NO: 19) |
| Optimised consensus | KCIMKEKKSLVRLSSCVPVALMSAMTTS |
| peptide | SSQKN (SEQ ID NO: 19) |

The optimised consensus peptide of TGFβR2 (SEQ ID NO: 19) was modified in order to overcome predicted difficulties with synthetic production and use as a vaccine. In particular, the optimised consensus sequence (SEQ ID NO: 19) is 33 amino acids long, and peptides of this length are difficult to produce synthetically to the appropriate quality and yield. The two cysteine residues in the same peptide create problems with the stability and quality of the peptide due to peptide cyclisation by formation of intra- and inter-molecular disulphide bonds. This peptide cyclisation may also reduce immunological potency of the peptide, for example by impairing effective antigen processing. This may potentially cause processing of unrelated T-cell epitopes. In addition, the peptide cyclisation may induce unwanted inflammatory side effects, for example, antibody formation or allergic reactions. Furthermore, the eight amino acids at the N-terminal of the optimised consensus sequence (SEQ ID NO: 19) correspond to the wild-type TGFβR2, such that there is a risk of activating wild-type cross-reactive T-cells. Consequently, the optimised consensus sequence (SEQ ID NO: 19) was further modified to overcome these issues, and the peptides shown in Table 5 were designed. FIG. 1 shows how the designed peptides relate to one another and to the optimised consensus sequence.

TABLE 5
Modified optimised peptides

| Peptide Name | Sequence |
|---|---|
| Fsp1 | KCIMKEKKSLVRLSSCVPVALMSA |
| (SEQ ID NO: 20) | |
| Fsp2 | KGIMKEKKSLVRLSSCVPVALMSA |
| (SEQ ID NO: 21) | |
| Fsp3 | SSCVPVALMSAMTTSSSQKN |
| (SEQ ID NO: 22) | |
| Fsp4 | SSGVPVALMSAMTTSSSQKN |
| (SEQ ID NO: 23) | |
| Fsp5 | KCIMKEKKSLVRLSSCVPVALMSAMTTSSSQKN |
| (SEQ ID NO: 19) | |
| Fsp6 | KCIMKEKKSLVRLSSCV |
| (SEQ ID NO: 24) | |
| Fsp6a | KGIMKEKKSLVRLSSCV |
| (SEQ ID NO: 25) | |

### Example 2—TGFβR2 Peptide Synthesis

i) Peptide Synthesis

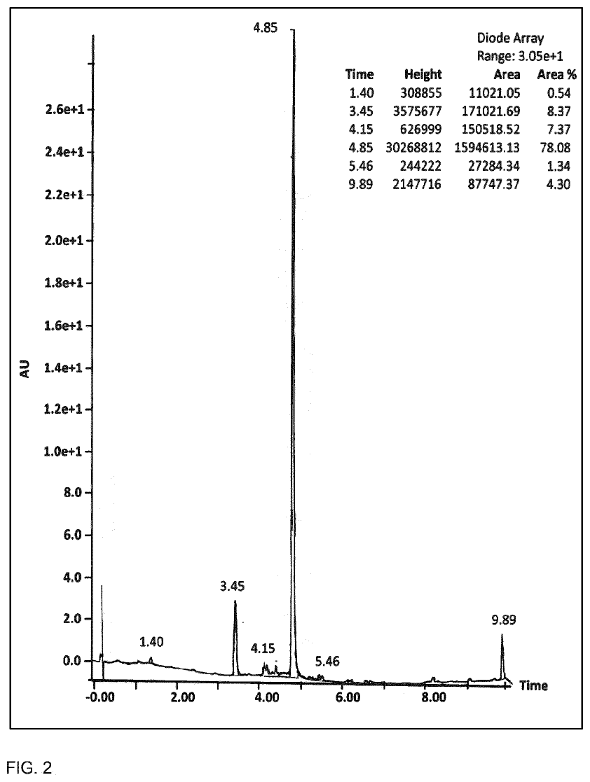

A batch of fsp5 (SEQ ID NO: 19) was prepared by solid phase peptide synthesis (SPPS) by using FMOC chemistry and Prelude synthesizer (Gyros Protein Technologies Inc., USA). The crude peptide was analysed by UPLC and MS. The desired peptide was observed by MS, and UPLC showed a purity of around 75% as demonstrated in FIG. 2. UPLC system: Column; Acquity UPLC BEH C18 1.7 mm, 21×150 mm, detection; PDA 210-500 nm, solvent A); 0.1% TFA in water, solvent B; 0.1% TFA in MeCN, gradient: 20-70% B (2-10 minutes, linear).

ii) Peptide Solubility and Purification

The peptide was difficult to dissolve and it was attempted to dissolve the crude fsp5 (SEQ ID NO: 19) (˜150 mg) in approximately 4 ml 50% MeCN in water under gentle heating. However, no clear solution could be obtained. Nevertheless, it was attempted to purify the crude solution by preparative HPLC (15-40% MeCN in water) after filtration of the crude suspension, which resulted in large losses of material. Only very small amounts of the desired peptide were obtained, and analysis by UPLC found that the peptide was impure.

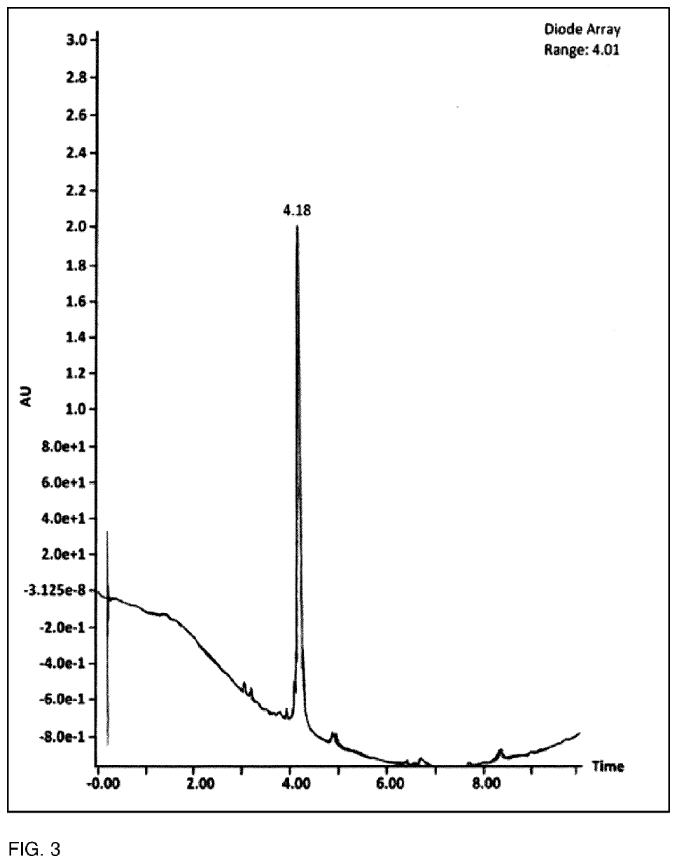

Another batch of fsp5 (SEQ ID NO: 19) was prepared, dissolved in neat DMSO under gentle heating and purified by preparative HPLC. The purity of the peptide was measured by UPLC, and was found to be about 90%, as shown in FIG. 3. Again, only very small amounts of purified fsp5 (SEQ ID NO: 19) were obtained. It was noted that the addition of even small amounts of water led to precipitation of the peptide in the crude DMSO solution, so it is very likely that fsp5 (SEQ ID NO: 19) precipitates on the column during HPLC purification.

iii) Peptide Purity and Stability

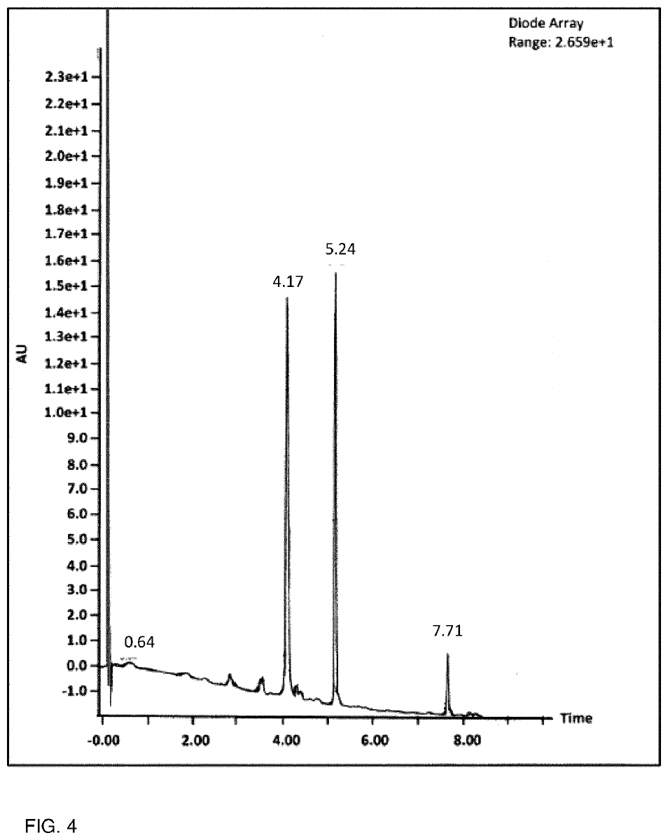

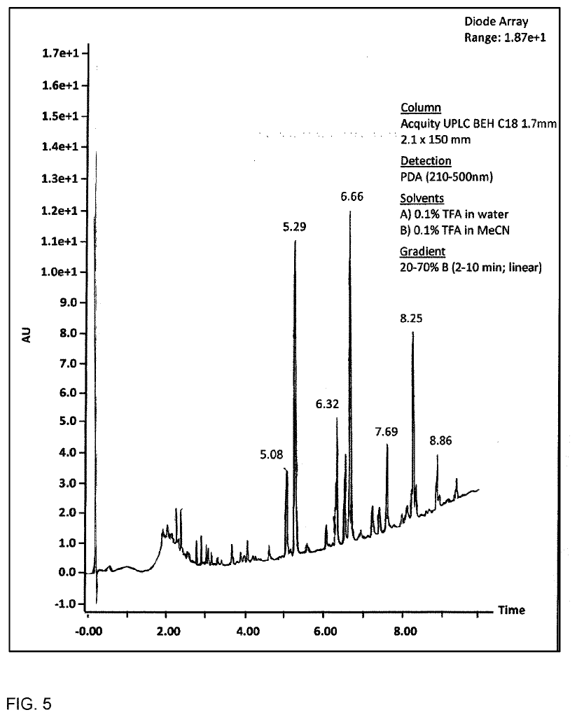

When the purity of the small amount of purified fsp5 (SEQ ID NO: 19) was reassessed by UPLC after lyophilisation, the purity had dropped from around 90% to below 50% as shown in FIG. 4. The same trend was observed when the purity of crude fsp5 (SEQ ID NO: 3) was reassessed after a short storage (3 days) at room temperature followed by lyophilization, as demonstrated by FIG. 5.

In summary, Example 2 shows that fsp5 (SEQ ID NO: 19) can be synthesized but is very difficult to produce in amounts sufficient for any practical purposes, and to a quality necessary for use in medicine, for example, as a constituent of a potential cancer vaccine.

### Example 3—TGFβR2 Peptide Synthesis

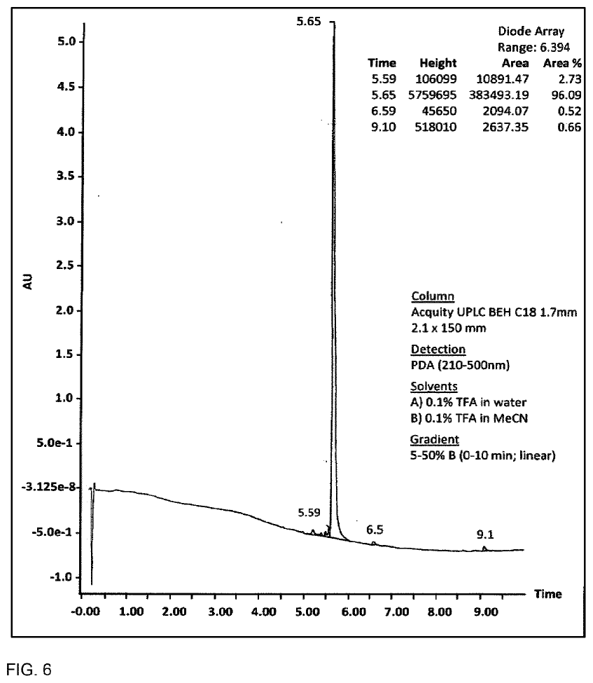

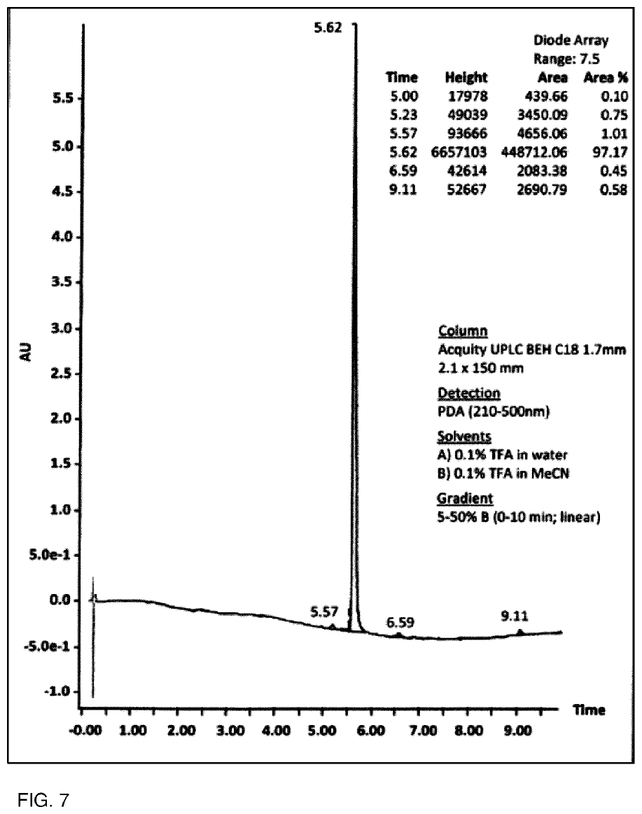

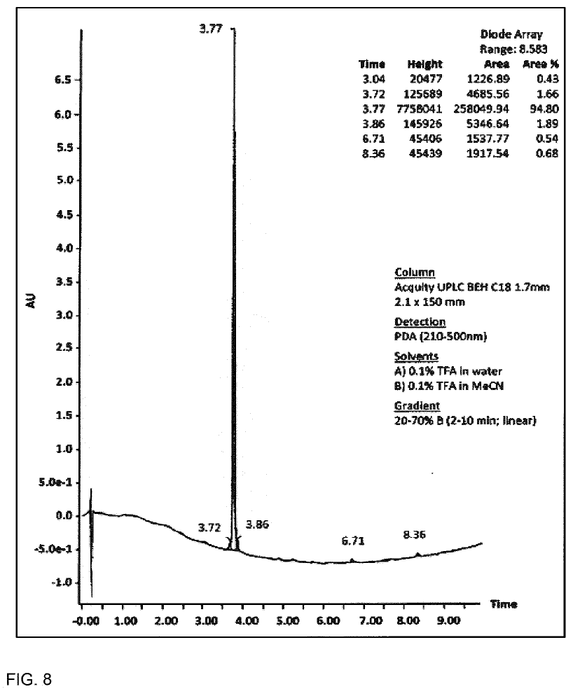

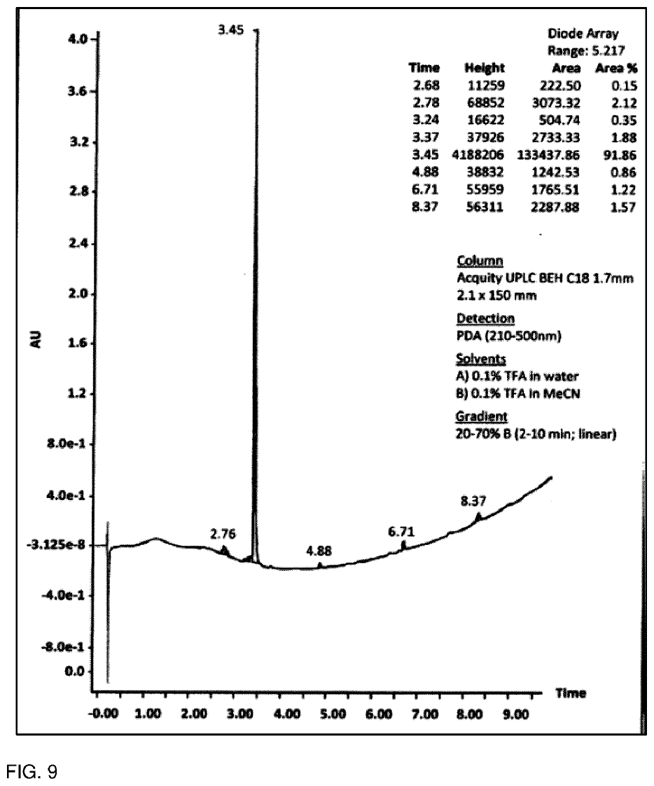

The Peptides fsp1 (SEQ ID NO: 20), fsp2 (SEQ ID NO: 21), fsp3 (SEQ ID NO: 22) and fsp4 (SEQ ID NO: 23), were synthesised by using SPPS and purified by HPLC as described above. The peptides were lyophilised after purification. The purity of each of the peptides was measured by UPLC and the UPLC traces are set out in FIGS. 6-9. UPLC system: Column; Acquity UPLC BEH C18 1.7 mm, 21×150 mm, detection; PDA 210-500 nm, solvent A); 0.1% TFA in water, solvent B; 0.1% TFA in MeCN, gradient for fsp1 and fsp2: 5-50% B (0-10 minutes, linear), gradient for fsp3 and fsp4: 20-70% B (2-10 minutes, linear).
FIG. 6 show that the obtained purity for purified and lyophilised fsp1 (SEQ ID NO: 20) was 96%.
FIG. 7 show that the obtained purity for purified and lyophilised fsp2 (SEQ ID NO: 21) was 97%.
FIG. 8 show that the obtained purity for purified and lyophilised fsp3 (SEQ ID NO: 22) was 94.8%.
FIG. 9 show that the obtained purity for purified and lyophilised fsp4 (SEQ ID NO: 23) was 91.9%.

Correct molecular weight (MW) was confirmed for all four peptides by ESI-QTOF-MS (Table 6, Example 4).

In summary, Example 3 demonstrates that the peptides fsp1 (SEQ ID NO: 20), fsp2 (SEQ ID NO: 21), fsp3 (SEQ ID NO: 22) and fsp4 (SEQ ID NO: 23) can be produced without the chemical problems seen with fsp5 (SEQ ID NO: 19). It is therefore feasible to produce these shorter peptides for potential use as vaccines to induce peptide specific T cells.

### Example 4—Immunogenicity of the TGFβR2 Peptides

i) Materials

TABLE 6
Materials

| Peptide | Purity | Sequence | MW | Salt | MW incl. salt |
|---|---|---|---|---|---|
| fsp1 | 96% | KCIMKEKKSLVRLSSCVP | 2622.34 | 6TFA | 3306.46 |
| | | VALMSA (SEQ ID NO: 20) | | | |
| fsp2 | 97% | KGIMKEKKSLVRLSSCVP | 2576.25 | 6TFA | 3260.37 |
| | | VALMSA (SEQ ID NO: 21) | | | |
| fsp3 | 94.8% | SSCVPVALMSAMTTSSS | 2029.36 | 2TFA | 2257.40 |
| | | QKN (SEQ ID NO: 22) | | | |
| fsp4 | 91.9% | SSGVPVALMSAMTTSSS | 1983.27 | 2TFA | 2211.38 |
| | | QKN (SEQ ID NO: 23) | | | |
| fsp5 | ~50% | KCIMKEKKSLVRLSSCVP | 3571.21 | 7TFA | 4369.39 |
| | | VALMSAMTTSSSQKN | | | |
| | | (SEQ ID NO: 19) | | | |

Fresh buffy coats from four healthy donors were obtained from a blood bank.

ii) First In Vitro Stimulation of PBMC and T-Cell Proliferation

Day 1:

PBMC:

Peripheral blood mononuclear cells (PBMC) isolated from fresh buffy coats from four healthy donors were counted and suspended in DC medium to 15*10⁶ cells/ml, and subsequently diluted with DC-medium to 4*10⁶ cells/ml (Table 6).

TABLE 7
Cell suspension and dilution

| | | | | Cell suspension | | | |
|---|---|---|---|---|---|---|---|
| | | | | 15 * 10⁶ cells/ml | | Dilution to | |
| | | | | Total | | 4 * 10⁶ cells/ml | |
| | Counted | Viability | | cells | DC | Cell | DC- |
| Donor | cells (*10⁶) | % | Dilution | (*10⁶) | medium | suspension | medium |
| 1 | 1.85 | 96 | 5x | 462 | 30.8 ml | 6.7 ml | 18.3 ml |
| 2 | 2.49 | 78 | 5x | 622 | 41.4 ml | 6.7 ml | 18.3 ml |
| 3 | 2.24 | 94 | 5x | 560 | 37.3 ml | 6.7 ml | 18.3 ml |
| 4 | 2.25 | 93 | 5x | 563 | 37.5 ml | 6.7 ml | 18.3 ml |

DC-medium: 500 ml CellGro DC medium (CellGenix) + 0.63 ml of 40 mg/ml Gensumycin + 5 ml 1M HEPES buffer + 4 ml of 200 mg/ml Mucomyst/NAC

In-Vitro Stimulation:

Four 24-well plates were used for each donor, with 4*10⁶ cells per well (each well has a volume of 1 ml).

Peptide cocktail: Solution of peptides fsp2+fsp4, containing 10 M of each peptide. 40 μl peptide solution was added to each well.

The plates were incubated in a cell incubator for 14 days (37° C., 5% CO₂). IL-2 and IL-7 were added on day 3. The cells were inspected daily.

Day 14—T Cell Proliferation:

Test peptides: fsp2+fsp4 (SEQ ID NOs: 21 and 23), fsp1 (SEQ ID NO: 20), fsp2 (SEQ ID NO: 21), fsp3 (SEQ ID NO: 22), fsp4 (SEQ ID NO: 23), fsp5 (SEQ ID NO: 19).

Positive control: SEC3

Negative controls: T cells, T cells+APC (without addition of test peptides)

Mock: DMSO in PBS

Plate Set Up for T Cell Proliferation:

Cells and reagents were added to plate wells (in triplicate) as described in Tables 8 and 9 below. The total volume added to each well was 0.20 ml.
T cells (PBMC): 50 000 cells/well (0.25*10⁶ cells/ml)
APC: Irradiated (30 Gy, 8 minutes) autologous feeder cells: 50 000 cells/well
(fspx): 0.2 nmol peptide/well (each peptide), concentration: 10 M peptide. (fsp was dissolved in DMSO before dilution to correct concentration)

TABLE 8
Plate 1 (96 wells) set up:

| Row A-H | Wells 1-3 | Wells 4-6 | Wells 7-9 | Wells 10-12 |
|---|---|---|---|---|
| A | DC-medium | DC-medium | DC-medium | DC-medium |
| B (donor 1) | T cells | T cells + | T cells + | T cells + |
| | | APC | APC + fsp1 | APC + fsp2 |
| C (donor 1) | T cells + | T cells + | T cells + | T cells + |
| | APC + fsp3 | APC + fsp4 | APC + fsp5 | APC + |
| | | | | fsp2 + fsp4 |
| D (donor 1) | T cells + | T cells + | medium | medium |
| | APC + mock | APC + SEC3 | | |
| E (donor 2) | T cells | T cells + | T cells + | T cells + |
| | | APC | APC + fsp1 | APC + fsp2 |
| F (donor 2) | T cells + | T cells + | T cells + | T cells + |
| | APC + fsp3 | APC + fsp4 | APC + fsp5 | APC + |
| | | | | fsp2 + fsp4 |
| G (donor 2) | T cells + | T cells + | medium | medium |
| | APC + mock | APC + SEC3 | | |
| H | medium | medium | medium | medium |

TABLE 9
Plate 2 (96 wells) set up:

| Row A-H | well 1-3 | well 4-6 | well 7-9 | well 10-12 |
|---|---|---|---|---|
| A | DC-medium | DC-medium | DC-medium | DC-medium |
| B (donor 3) | T cells | T cells + | T cells + | T cells + |
| | | APC | APC + fsp1 | APC + fsp2 |
| C (donor 3) | T cells + | T cells + | T cells + | T cells + |
| | APC + fsp3 | APC + fsp4 | APC + fsp5 | APC + |
| | | | | fsp2 + fsp4 |
| D (donor 3) | T cells + | T cells + | medium | medium |
| | APC + mock | APC + SEC3 | | |
| E (donor 4) | T cells | T cells + | T cells + | T cells + |
| | | APC | APC + fsp1 | APC + fsp2 |
| F (donor 4) | T cells + | T cells + | T cells + | T cells + |
| | APC + fsp3 | APC + fsp4 | APC + fsp5 | APC + |
| | | | | fsp2 + fsp4 |
| G (donor 4) | T cells + | T cells + | medium | medium |
| | APC + mock | APC + SEC3 | | |
| H | medium | medium | medium | medium |

The plates were incubated in a cell incubator for 48 hours (37° C., 5% CO₂).

Day 16.

After incubation for 48 hours, 20 μl 3H-tThymidine solution (5 Ci/ml) was added to each well and the plates were incubated for approximately 17 hours (37° C., 5% CO₂).

Day 17.

After 17 hours the cells were harvested (Unifilters) and dried on the filters at 45° C. until completely dry. After covering the bottom of the Unifilters with adhesive covers (delivered with the Unifilters) 25 μl micro scintillation liquid was added to each well, the plate was covered with TopSeal and 3H-Thymidine uptake was measured as counts per minute (CPM) using a microplate scintillation counter.

Proliferation Results:

The T cell proliferation results after the first round of in vitro stimulation with the cocktail of fsp2 and fsp4 peptides are presented as stimulation index (SI) in FIG. 10. SI=mean CPM (triplicates) of (T cells+APC+test peptide(s)) divided by mean CPM (triplicates) of (T cells+APC). SI>1 indicates an increase in T-cell proliferation and SI 2 is a clear sign of immunogenicity.

iii) In Vitro Re-Stimulation of PBMC and T-Cell Proliferation

PBMCs harvested after the first in vitro stimulation were re-stimulated in vitro according to the protocol set out above, with 2×10⁶ cells per well. T-cell proliferation was tested according to the protocol set out above.

Proliferation Results:

The T cell proliferation results after a second round of in vitro stimulation with the cocktail of fsp2 and fsp4 peptides are presented as stimulation index (SI) in FIG. 11.

Thus, FIGS. 10 and 11 show that the peptides fsp2 and fsp4, having an amino acid substitution, are immunogenic and can activate T-cells, and that the activated T-cells are cross-reactive for peptides of the naturally occurring −1a frameshifted TGFβ2 protein. In addition, FIGS. 10-12 show that fsp1 and fsp5, which are peptides of the naturally-occurring −1a frameshifted TGFβ2 protein, stimulated T-cells induced from PMBCs and, therefore, are immunogenic. Consequently, the modified peptides fsp2 (SEQ ID NO. 21) and fsp4 (SEQ ID NO. 23), as well as unmodified peptides fsp1 (SEQ ID NO: 20) and fsp5 (SEQ ID NO: 19), can be used to stimulate induction of TGFβR2 frameshift mutant-specific T-cells.

### Example 5—Immunogenicity of fsp6 and fsp6a

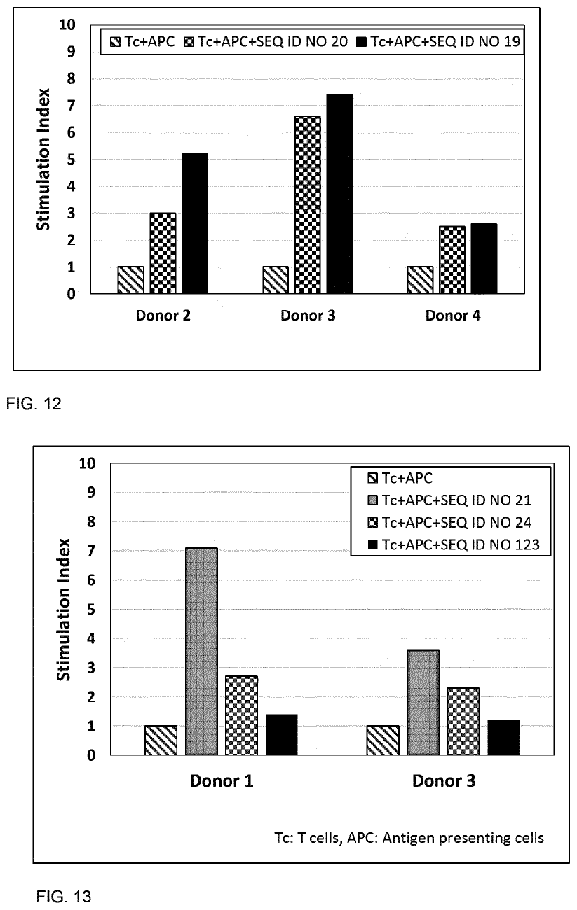

A similar protocol to that set out in Example 4 was used to test the immunogenicity of fsp6 (SEQ ID NOs: 24) and fsp7 (SEQ ID NO: 123). Fsp2 (SEQ ID NO: 21; 10 μM) was used to induce T-cells from PMBCs isolated from fresh buffy coats from four healthy donors. At day 14 after induction, a T-cell proliferation assay was carried out as in Example 4, using fsp2 (SEQ ID NO: 21), fsp6 (SEQ ID NO: 24) and fsp7 (SEQ ID NO: 123) as test peptides. The T-cell proliferation results after one round of in vitro stimulation are presented as stimulation index (SI) in FIG. 13. In particular, FIG. 13 shows that fsp6 is capable of activating T-cells, such that fsp6 is immunogenic and can be used as a vaccine or treatment against cancer. FIG. 13 also shows that T-cells induced by fsp2 (SEQ ID NO: 21) are cross-reactive for peptides of the naturally-occurring TGFβR2-1a frameshift protein which are shorter than fsp2 (SEQ ID NO: 21). It is also expected, in view of the results shown in FIGS. 11 and 12, that a peptide having the sequence of fsp6 (SEQ ID NO: 26) but having a C-to-G amino acid substitution corresponding to that in fsp2 is also immunogenic and is useful as a vaccine or treatment against cancer. In particular, as mentioned in Example 4, FIG. 12 shows that T-cells induced by fsp2 (SEQ ID NO: 21), which has a C-to-G amino acid substitution, are cross-reactive for peptides of the naturally-occurring TGFβR2-1a frameshift mutant protein, such that the C-to-G amino acid substitution is immunologically acceptable. Thus, it is expected that fsp6a (SEQ ID NO: 25), which is identical to fsp6 (SEQ ID NO: 24) except for the same C-to-G amino acid substitution as fsp2 (SEQ ID NO: 21), will also be immunogenic.

Furthermore, FIG. 13 shows that fsp6 (SEQ ID NO: 24) is less immunogenic than fsp2 (SEQ ID NO: 21), but that both fsp6 and fsp2 are much more immunogenic than fsp7 (SEQ ID NO: 123). Fsp6 has the same amino acid sequence as fsp2, except that fsp6 is truncated at the C-terminus compared to fsp2 and does not have a C-to-G amino acid substitution. Fsp7 has the same amino acid sequence as fsp2 but is truncated at the N-terminus compared to fsp2. In view of the difference in immunogenicity between these three peptides, shown in FIG. 13, it is expected that at least one additional amino acid at the C-terminus of fsp6 is required in order to retain the immunogenicity of fsp2. In addition, it is expected that at least one additional amino acid at the N-terminus of fsp7 is required in order to increase its immunogenicity and for the peptide to comprise immunologically effective epitopes. Consequently, it is expected that fsp1a (LVRLSSCVP; SEQ ID NO: 124) is immunogenic, or is a significant element of an immunogenic peptide, as this peptide comprises a sequence shared by fsp6 and fsp7, with the addition of one amino acid at the C-terminus compared to fsp6 and one amino acid at the N-terminus compared to fsp7. Furthermore, as the C-to-G amino acid substitution has been shown to be immunologically acceptable, it is expected that fsp1a having a C-to-G amino acid substitution (i.e. fsp1b; SEQ ID NO: 125) will also be immunogenic.

### Example 6-ASTE1, TAF13, KIAA2018 and SLC22A9 Peptides

Clusters (nested epitopes) of potential HLA class II T-cell epitopes (15-mers) were identified using the online algorithm SYFPEITHI, using a SYFPEITHI cut off score of ≥20. A predicted nested epitope peptide was defined, and potential HLA class I epitopes (9-mers) were predicted using SYFPEITHI and a SYFPEITHI cut off score of ≥20. All HLA class I and HLA class-II alleles provided for by SYFPEITHI were included in the searches. Candidate fsp peptides were designed based on an overall consideration of:
Total HLA-coverage with main focus on HLA-class II
Prevalence of the various HLA alleles in the general population
A representative peptide of each protein for in vitro testing was defined
Chemical considerations: try to avoid multiple cysteine residues, potential issues for synthesis, solubility, and stability of the potential candidate peptides

The predicted HLA class II epitopes, predicted nested epitope peptide and HLA class I epitopes, and the candidate fsp peptide, for each −1a frameshift mutant protein are shown in Tables 10-13 below. With regard to Tables 12 and 13, the search sequences shown therein (i.e. SEQ ID NOs: 76 and 96) were considered to be the most relevant for identification of predicted epitopes.

TABLE 10
ASTE1-1a frameshift mutant protein

| | | |
|---|---|---|
| Search sequence | GRSNSKKKGRRNRIPAVLRTEGEPLHTPSVGMRETTGLGC | |
| | (SEQ ID NO: 50) | |
| HLA-class II | EGEPLHTPSVGMRET (SEQ ID NO: 51) | HLA-DRB1*0701 |
| epitopes | RTEGEPLHTPSVGMR (SEQ ID NO: 52) | HLA-DRB1*0101 |
| (15 mers) | IPAVLRTEGEPLHTP (SEQ ID NO: 53) | HLA-DRB1*0101, |
| | | HLA-DRB1*0301, |
| | | HLA-DRB1*0701 |
| | RNRIPAVLRTEGEPL (SEQ ID NO: 54) | HLA-DRB1*0401, |
| | | HLA-DRB1*1101 |
| Predicted nested | RNRIPAVLRTEGEPLHTPSVGMRET (SEQ ID NO: 55) | |
| epitope peptide | | |
| HLA-class I | TEGEPLHT (SEQ ID NO: 56) | HLA-B*41:01, HLA-B*45:01 |
| epitopes | LRTEGEPLH (SEQ ID NO: 57) | HLA-B*27:05 |
| (9 mers) | VLRTEGEPL (SEQ ID NO: 58) | HLA-A*02:01, HLA-B*08 |
| Candidate | RNRIPAVLRTEGEPLHTPSVGMRET (SEQ ID NO: 26) | HLA-DRB1*0101, |
| peptide fsp8 | | HLA-DRB1*0301, |
| | | HLA-DRB1*0401, |
| | | HLA-DRB1*0701, |
| | | HLA-DRB1*1101, HLA-A*02:01, |
| | | HLA-B*08, HLA-B*27:05, |
| | | HLA-B*41:01, HLA-B*45:01 |

TABLE 11
TAF1β-1a frameshift mutant protein

| | | |
|---|---|---|
| Search sequence: | QIKALNRGLKKKTILKKAGIGMCVKVSSIFFINKQKP | |
| | (SEQ ID NO: 59) | |
| HLA-class II | CVKVSSIFFINKQKP (SEQ ID NO: 60) | HLA-DRB1*0401, HLA- |
| epitopes | | DRB1*1501 |
| (15 mers) | GMCVKVSSIFFINKQ (SEQ ID NO: 61) | HLA-DRB1*0701, HLA- |
| | | DRB1*1501 |
| | IGMCVKVSSIFFINK (SEQ ID NO: 62) | HLA-DRB1*0101 |
| | GIGMCVKVSSIFFIN (SEQ ID NO: 63) | HLA-DRB1*0101, HLA- |
| | | DRB1*0701 |
| | KAGIGMCVKVSSIFF (SEQ ID NO: 64) | HLA-DRB1*1101, HLA- |
| | | DRB1*1501 |
| | KKTILKKAGIGMCVK (SEQ ID NO: 65) | HLA-DRBV0101, HLA- |
| | | DRB1*1501 |
| | LKKKTILKKAGIGMC (SEQ ID NO: 66) | HLA-DRB1*0101 |
| | GLKKKTILKKAGIGM (SEQ ID NO: 67) | HLA-DRB1*1101 |
| | NRGLKKKTILKKAGI (SEQ ID NO: 68) | HLA-DRB1*1501 |
| Predicted nested | NRGLKKKTILKKAGIGMCVKVSSIFFINKQKP | |
| epitope peptide | (SEQ ID NO: 69) | |
| HLA-class I | SIFFINKQK (SEQ ID NO: 70) | HLA-A*03, HLA-A*11:01 |
| epitopes | VSSIFFINK (SEQ ID NO: 71) | HLA-A*11:01 |
| (9 mers) | GMCVKVSSI (SEQ ID NO: 72) | HLA-A*02:01 |
| | AGIGMCVKV (SEQ ID NO: 73) | HLA-A*02:01 |
| | KAGIGMCVK (SEQ ID NO: 4) | HLA-A*03 |
| | KTILKKAGI (SEQ ID NO: 75) | HLA-A*02:01 |
| Candidate | KTILKKAGIGMCVKVSSIFFINKQK | HLA-DRB1*0101, HLA- |
| peptide fsp9 | (SEQ ID NO: 27) | DRB1*0401, HLA-DRB1*0701, |
| | | HLA-DRB1*1101, HLA- |
| | | DRB1*1501, HLA-A*02:01, |
| | | HLA-A*03, HLA-A*11:01, HLA- |
| | | B*15:16, HLA-B*58:02 |

TABLE 12
KIAA2018-1a frameshift mutant protein

| | | |
|---|---|---|
| Search sequence: | TLLSDLAKKKTLRNHLFLIRWIILTFLQKILK | |
| | (SEQ ID NO: 76) | |
| HLA-class II | LIRWIILTFLQKILK (SEQ ID NO: 77) | HLA-DRB1*0101, HLA- |
| epitopes | | DRB1*0401 |
| (15 mers) | LFLIRWIILTFLQKI (SEQ ID NO: 78) | HLA-DRB1*0101, HLA- |
| | | DRB1*0401, HLA-DRB1*0701 |
| | HLFLIRWIILTFLQ (SEQ ID NO: 79) | HLA-DRB1*0301 |
| | RNHLFLIRWIILTFL (SEQ ID NO: 80) | HLA-DRB1*0101, HLA- |
| | | DRB1*0401, HLA-DRB1*0701 |
| | KKTLRNHLFLIRWII (SEQ ID NO: 81) | HLA-DRB1*1501 |
| Predicted nested | KKTLRNHLFLIRWIILTFLQKILK (SEQ ID NO: 82) | |
| epitope peptide | | |
| HLA class I | LTFLQKILK (SEQ ID NO: 83) | HLA-A*11:01, HLA-A*68:01 |
| epitopes (9 mers) | ILTFLQKIL (SEQ ID NO: 84) | HLA-A*02:01, HLA-B*13 |
| | IILTFLQKI (SEQ ID NO: 85) | HLA-A*02:01 |
| | WIILTFLQK (SEQ ID NO: 86) | HLA-A*03, HLA-A*11:01 |
| | IRWIILTFL (SEQ ID NO: 87) | HLA-B*27:05, HLA-B*27:09, |
| | | HLA-B*39:01 |
| | LIRWIILTF (SEQ ID NO: 88) | HLA-A*03, HLA-A*26 |
| | FLIRWIILT (SEQ ID NO: 89) | HLA-A*02:01 |
| | LFLIRWIIL (SEQ ID NO: 90) | HLA-B*37 |
| | NHLFLIRWI (SEQ ID NO: 91) | HLA-B*39:01 |
| | LRNHLFLIR (SEQ ID NO: 92) | HLA-B*27:05 |
| | TLRNHLFLI (SEQ ID NO: 93) | HLA-A*02:01 |
| | KTLRNHLFL (SEQ ID NO: 94) | HLA-B*14:02, HLA-B*58:02 |
| | KKTLRNHLF (SEQ ID NO: 95) | HLA-B*39:02 |
| Candidate | KKTLRNHLFLIRWIILTFLQKILK (SEQ ID NO: 28) | HLA-DRB1*0101, HLA- |
| peptide fsp10 | | DRB1*0301, HLA-DRB1*0401, |
| | | HLA-DRB1*0701, HLA- |
| | | DRB1*1501, HLA-A*02:01, |
| | | HLA-A*03, HLA-A*11:01, HLA- |
| | | A*26, HLA-A*68:01, HLA-B*13, |
| | | HLA-B*14:02, HLA-B*27:05, |
| | | HLA-B*27:09, HLA-B*37, HLA- |
| | | B*39:01, HLA-B*39:02, HLA- |
| | | B*58:02 |

TABLE 13
SLC22A9-1a frameshift mutant protein

| Search | KKNLLCVKCSTCPTYVKGSPSCPLRDLQTLWPILALISMSSIWGTMFSCCRLSLVQ | |
|---|---|---|
| sequence: | SSSWPTVLHLGH (SEQ ID NO: 96) | |
| HLA-class II | (SEQ ID NO: 97) WGTMFSCCRLSLVQS | HLA-DRB1*0301 |
| epitopes | ISMSSIWGTMFSCCR (SEQ ID NO: 98) | HLA-DRB1*1501 |
| (15 mers) | LISMSSIWGTMFSCC (SEQ ID NO: 99) | HLA-DRB1*0101, |
| | | HLA-DRB1*1501 |
| | ILALISMSSIWGTMF (SEQ ID NO: 100) | HLA-DRB1*0101 |
| | WPILALISMSSIWGT (SEQ ID NO: 101) | HLA-DRB1*0101, |
| | | HLA-DRB1*0401, |
| | LWPILALISMSSIWG (SEQ ID NO: 102) | HLA-DRB1*1501 |
| | QTLWPILALISMSSI (SEQ ID NO: 103) | HLA-DRB1*0101, |
| | | HLA-DRB1*0401, |
| | | HLA-DRB1*1501 |
| | LQTLWPILALISMSS (SEQ ID NO: 104) | HLA-DRB1*0101, |
| | | HLA-DRB1*1501 |
| | LRDLQTLWPILALIS (SEQ ID NO: 105) | HLA-DRB1*0101, |
| | | HLA-DRB1*1501 |
| | SCPLRDLQTLWPILA (SEQ ID NO: 106) | HLA-DRB1*0101, |
| | | HLA-DRB1*1501 |
| | PTYVKGSPSCPLRDL (SEQ ID NO: 107) | HLA-DRB1*0101, |
| | | HLA-DRB1*0701 |
| | CPTYVKGSPSCPLRD (SEQ ID NO: 108) | HLA-DRB1*0101 |
| | STCPTYVKGSPSCPL (SEQ ID NO: 109) | HLA-DRB1*0101 |
| | KNLLCVKCSTCPTYV (SEQ ID NO: 110) | HLA-DRB1*0101 |
| Predicted | SCPLRDLQTLWPILALISMSSIWGTMFS (SEQ ID NO: 111) | |
| nested | STCPTYVKGSPSCPLRDLQ (SEQ ID NO: 112) | |
| epitope | | |
| peptides | | |
| HLA class I | LALISMSSI (SEQ ID NO: 113) | HLA-B*51:01 |
| epitopes (9 | TLWPILALI (SEQ ID NO: 114) | HLA-A*02:01, HLA- |
| mers) | | B*13 |
| | QTLWPILAL (SEQ ID NO: 115) | HLA-A*02:01, HLA- |
| | | A*26 |
| | CPLRDLQTL (SEQ ID NO: 116) | HLA-B*51:01 |
| | SPSCPLRDL (SEQ ID NO: 117) | HLA-B*07:02 |
| | CSTCPTYVK (SEQ ID NO: 18) | HLA-B*11:01 |
| | (SEQ ID NO: 119) SSWPTVLHL | HLA-B*58:02 |
| | (SEQ ID NO: 120) LSLVQSSSW | HLA-B*58:02 |
| | (SEQ ID NO: 121)RLSLVQSSS | HLA-A*03 |
| | FSCCRLSLV (SEQ ID NO: 122) | HLA-B*15:16 |
| Candidate | fsp11: SCPLRDLQTLWPILALISMSSIWGTMFS (SEQ ID NO: 29) | HLA-DRB1*0101, |
| peptides | fsp12: SGPLRDLQTLWPILALISMSSIWGTMFS (SEQ ID NO: 30) | HLA-DRB1*0401, |
| fsp11 and | | HLA-DRB1*1501, |
| fsp12 | | HLA-A*02:01, HLA- |
| | | A*26, HLA- |
| | | B*51:01, HLA-B*13 |
| Candidate | fsp13: STCPTYVKGSPSCPLRDLQ (SEQ ID NO: 31) | HLA-DRB1*0101, |
| peptide | fsp14: STGPTYVKGSPSCPLRDLQ (SEQ ID NO: 32) | HLA-DRB1*0701, |
| fsp13 and | | HLA-B*07:02, HLA- |
| 14 | | B*11:01 |

Fsp12 and fsp14 were designed with a C-to-G amino acid substitution (shown underlined in Table 13) for the same reasons as fsp2 and fsp4, discussed in Example 1, above. The cysteine residue closest to the N-terminus of the peptide was substituted, as it is considered that the amino acids closest to the N-terminus may not be essential for specific recognition by T-cells.

### Example 7—Immunogenicity of Peptides and Peptide Mixtures

i) Equipment
Heraeus megafuge 2.0
KOJAIR laminar flow hood KR-210 K-Safety
CO2 incubator, Forma Scientific Model 3111
FACSCanto

ii) Reagents
CellGro DC medium (Cat. no. 0020801-0500, CellGenix GmbH, Freiburg, Germany)
HEPES 1M (Fischer scientific)
Mucomyst/NAC (Meda AS, Asker, Norway)
Gensumycin (Gibco, Life Technologies)
IL-2
rhiL-7
SEC-3 superantigen 10 mg/ml stock (Toxin Technology Inc, USA)
Microplate 96-well, round bottomed (VWR International AS, Oslo, Norway)
Topseal-A (Cat. No. 6005185, Nerliens Meszansky, Oslo, Norway)
Microscient-0 scintillation liquid (Nerliens Meszansky, Oslo, Norway)
3H-Thymidine (Motebello Diagnostics, Oslo, Norway)
RPMI-1640 with L-gutamin (Gibco, Life Technologies)
Albunorm 200 g/l (20%) (Octapharma)
Dimethylsulfoxide (DMSO) (Sigma Aldrich)
Complete CellGro DC medium used for cell cultures
   The following was added to 500 ml of CellGro DC medium:
      1. Gensumycin; 630 μL of 40 mg/ml stock solution (final concentration 0.05 mg/ml)
      2. HEPES buffer; 5 ml of 1M stock stock solution (final concentration 0.01M)
      3. Mucomyst/NAC; 4 ml of 200 mg/ml stock solution (final concentration 1.6 ml/ml)

iii) Peptide Stimulation of PBMC Bulk Cultures

PBMCs isolated from buffy coats from four healthy donors (obtained from a blood bank) were suspended in complete CellGro DC medium to 15*10⁶ cells/ml and diluted to 4*10⁶ cells/ml (Table 14).

TABLE 14

| | | | | Dilution to 4 * 10⁶ PBMC/ml | | |
|---|---|---|---|---|---|---|
| | | PBMC suspension | | (suspension for in vitro stimulation) | | |
| | | 15 * 10⁶ cells/ml | | | | 500 μM |
| | Viability | Total | | | | peptide |
| | counted | cells | DC | Cell | | cocktail* |
| Donor | cells (*10⁶) | (*10⁶) | medium | suspension | DC-medium | (H₂0) |
| 1 | 49% | 300 | 20 ml | 6.4 ml | 16.2 ml | 1.44 ml |
| 2 | 86% | 725 | 48 ml | 6.4 ml | 16.2 ml | 1.44 ml |
| 3 | 43% | 725 | 48 ml | 6.4 ml | 16.2 ml | 1.44 ml |
| 4 | 44% | 575 | 38 ml | 6.4 ml | 16.2 ml | 1.44 ml |

*a stock solution of 166.67 μM/each peptide (fsp2, fsp8 and fsp9) was added to the wells, giving a final concentration of ≈10 μM/each peptide

On day 1, 1 ml of PBMC suspension (4×10⁶ cells/ml) from each donor was transferred to wells of 24-well plates (4*10⁶ cells per well) and 60 μl of the peptide cocktail (fsp2+fsp8+fsp9) stock solution (166.67 μM/each peptide) was added to each well. The plates were incubated in a cell incubator for 14 days. IL-2 and IL-7 was added on day 3 (0.50 ml DC medium containing 60 U/ml IL-2 and 15 ng/ml IL-7). The cells were inspected daily and DC medium with cytokines (IL-2, IL7) was refreshed according to in house laboratory routines.

The in vitro stimulation of bulk cultures was repeated a further two times (i.e. a total of three stimulations) using PBMCs harvested after the preceding round of in vitro stimulation. Peptide specific T-cell proliferation was tested on day 14, after each round of stimulation, such that T-cell proliferation was tested on days 14, 28 and 42 after the first round of stimulation.

iv) Three-Day T-Cell Proliferation Assay

The cells from the bulk cultures were harvested (5 minutes at 300×g centrifugation) and re-suspended in DC medium (5 ml). An aliquot of the 5 ml resuspension was diluted 5 times and the T-cells counted in order to calculate the volume required to provide 5×10⁴ cells per well for the proliferation assay. T-cells from each donor were transferred (triplicates) to wells of 96-well plates (5×10⁴ cells per well) together with autologous, irradiated (30 Gy for 8 minutes) PBMCs (5×10⁴ cells per well) and test peptide(s) (final concentration 10 μM/each peptide). Negative controls were wells without test peptide(s) and positive controls were wells with SEC-3 (0.1 μg/ml) without test peptide(s). The cells (plates) were incubated for 48 hours (37° C./5% CO₂). After 48 hours, 20 μL of 3H-Thymidine (3.7×10₄ Bq) was added to the cells and incubation was continued for 17 hours. The cells were harvested (Unifilters using the Filtermate 196 Harvester) and dried on the filters for approx. 4.5 hours at 45° C. The cells were counted (CPM) by using the standard in-house laboratory protocol (TopCount Packard microplate scintillation beta counter and run programme).

v) Peptide-Specific T-Cell Proliferation

One Stimulation:

Peptide-specific T-cell proliferation was measured after 14 days of stimulation of PBMC bulk cultures with a peptide cocktail containing fsp2, fsp8 and fsp9 (10 μM each peptide). Proliferation induced by each of the single peptides and the peptide cocktail was tested at a concentration of 10 M (each peptide).

Two Stimulations:

Peptide-specific T-cell proliferation was measured after 14 days of re-stimulation of PBMC with a peptide cocktail containing fsp2, fsp8 and fsp9 (10 M each peptide). T-cell proliferation induced by each of the single peptides and the peptide cocktail was tested. The single peptides were tested at a concentration of 10 μM. The peptide cocktail was tested using two different amounts of each peptide in the cocktail, namely, a peptide cocktail containing 10 M each peptide and a peptide cocktail containing 3.33 μM each peptide.

Three Stimulations:

Peptide-specific T-cell proliferation was measured after 14 days of a further re-stimulation of PBMC with a peptide cocktail containing fsp2, fsp8 and fsp9 (10 M each peptide). T-cell proliferation induced by each of the single peptides and the peptide cocktail was tested. The single peptides were tested at a concentration of 10 μM. The peptide cocktail was tested using two different amounts of each peptide in the cocktail, namely, a peptide cocktail containing 10 μM each peptide and a peptide cocktail containing 3.33 μM each peptide.

The results of the peptide-specific T-cell proliferation assays are shown in FIGS. 14-18. In particular, an increase in the SI value compared to the control shows that the peptide cocktail or specific peptide is capable of inducing a T-cell response, while a SI≥2 is a clear signal of immunogenicity. FIG. 14 shoes that after two rounds of stimulation with the peptide cocktail, T-cells were induced against the peptide cocktail in three out of four donors. FIG. 15 shows that the T-cell response to the peptide cocktail after three rounds of stimulation was stronger than after 2 rounds of stimulation. FIGS. 16-18 show the T-cell response to each of the peptide cocktail, fsp2, fsp8 and fsp9, in three donors, and shows that all of these peptides and peptide mixtures are able to induce a T-cell response. More particularly, fsp2 has a SI greater than 2 in all three donors, fsp8 and fsp9 each has a SI greater than 2 in two out of three donors, and the peptide cocktail has a SI greater than 2 in all three donors. This indicates that each of the peptides is immunogenic, even when administered as a peptide cocktail. Furthermore, as each of fsp2, fsp8 and fsp9 is longer than HLA class-I epitopes (9-mers) and HLA class-II epitopes (15-mers), and were designed to include more than one epitope, the peptides encompass fragments which are expected to be immunogenic.

### Example 8

Using a similar procedure to that used in Examples 4 and 7 above, PMBCs from four healthy donors were stimulated in vitro with a peptide mixture containing equimolar amounts (10 uM) of fsp10 (SEQ ID NO: 28), fsp11 (SEQ ID NO: 29) and fsp13 (SEQ ID NO: 31).

After one round of stimulation (14 days), T-cells were harvested and tested for recognition of the peptide mixture, using single peptide concentrations of 10 μM. T-cell proliferation was measured as counts per minute (CPM) after uptake of ³H-thymidine in a standard T cell proliferation assay.

The proliferation test results indicated that T-cells capable of recognising the peptide mixture had been induced in donor 1. Retesting of new samples of harvested T-cells confirmed the findings of the first proliferation testing. In addition, the results showed that after one round of in vitro stimulation, fsp11 (SEQ ID NO: 29) and a mixture of fsp11 (SEQ ID NO: 29) and fsp13 (SEQ ID NO: 31) induced T-cell responses (FIG. 19). Fsp10 (SEQ ID NO: 28) and fsp13 (SEQ ID NO: 31), as individual peptides, did not stimulate T-cells in donor 1 after one round of in vitro stimulation. Fsp11 (SEQ ID NO: 29) alone showed a greater induction of T-cells than the mixture of fsp11 (SEQ ID NO: 29) and fsp13 (SEQ ID NO: 31; FIG. 19), and a possible explanation is that fsp13 (SEQ ID NO: 31) occupies HLA molecules on the surface of the antigen-presenting cells (APCs), thereby reducing the number of HLA molecules available to bind, and present to T-cells, fsp11 (SEQ ID NO: 29).

The PMBCs from the four healthy donors underwent a second round of in vitro stimulation, and T-cells were again harvested and tested for proliferation against the peptide mixture and the individual peptides. The proliferation results showed that T-cells are obtained from Donor 4 which respond to the peptide mixture in a concentration-dependent manner (FIG. 20). In particular, FIG. 20 shows that the SI value of the peptide mixtures is increased compared to the control (TC+APC), indicating a T-cell response to the peptide mixtures. While it has, in the past, been considered that a SI value of greater than 2 is the criterion for indicating a T-cell response in a T-cell proliferation assay, this is only the case in subjects who have been vaccinated prior to the T-cell proliferation assay. Where a healthy subject has not been vaccinated before the T-cell proliferation assay (as in the present T-cell proliferation assay), it is not expected that a SI value of 2 or greater will be achieved. Therefore, in non-vaccinated, healthy subjects, an increase in the SI value above 1 is indicative of a T-cell response.

### Example 9

Using a similar procedure to that used in Examples 4 and 7 above, PMBCs from four healthy donors were stimulated in vitro with a peptide mixture containing equimolar amounts (10 uM) of fsp17 (SEQ ID NO: 126), fsp15 (SEQ ID NO: 127) and fsp16 (SEQ ID NO: 128), which are shown in Table 15, below. These peptides have the non-chiral amino acid glycine at their C-terminal, which can serve as a linker for conjugation of the peptides to other molecules. It is expected that this C-terminal glycine will not create any new T-cell epitopes which will change the specificity of the induced T-cells.

TABLE 15

| | |
|---|---|
| Fsp15 (ASTE1) | KKKGRRNRIPAVLRTEGEPLHTPSVGMRETG |
| | (SEQ ID NO: 127) |
| Fsp16 (TAF1β) | GLKKKTILKKAGIGMCVKVSSIFFINKQKG |
| | (SEQ ID NO: 128) |
| Fsp17 (TGFβR2) | KGIMKEKKSLVRLSSCVPVALMSAMTG |
| | (SEQ ID NO: 126) |

After one round (14 days) of in vitro stimulation, T-cells were harvested and tested for recognition of the peptide mixture or individual peptides, using a concentration of 10 μM of each peptide. T-cell proliferation was measured as counts per minute (CPM) after uptake of ³H-thymidine in a standard T cell proliferation assay.

The proliferation test results showed that T-cells are induced by fsp15 (SEQ ID NO: 127; FIG. 21).

The PMBCs from the four healthy donors underwent a second round of in vitro stimulation, and T-cells were again harvested and tested for proliferation against the peptide mixture and the individual peptides. The results show that T-cells were induced by the peptide mixture (fsp17 (SEQ ID NO: 126), fsp15 (SEQ ID NO: 127) and fsp16 (SEQ ID NO: 128), and by fsp15 (SEQ ID NO: 127) and fsp17 (SEQ ID NO: 126), individually (FIG. 22).

1-23. (canceled)

24. A peptide selected from:
a peptide capable of inducing an immune response against a ASTE1-1a frameshift mutant protein, wherein the peptide comprises an immunogenic fragment of SEQ ID NO:5, wherein the fragment comprises at least 10 consecutive amino acids of SEQ ID NO:26, and wherein the peptide comprises at least 22 amino acids and no more than 31 amino acids;
a peptide capable of inducing an immune response against a TAF1β-1a frameshift mutant protein, wherein the peptide comprises an immunogenic fragment of SEQ ID NO:7, wherein the fragment comprises at least 12 consecutive amino acids of SEQ ID NO:27, and wherein the peptide comprises no more than 27 amino acids;
a peptide capable of inducing an immune response against a KIAA2018-1a frameshift mutant protein, wherein the peptide comprises a immunogenic fragment of any one of SEQ ID NOs:9-12 comprising at least 8 consecutive amino acids thereof, including at least one amino acid from positions 13 to 37 of SEQ ID NO:9, at least one amino acid from positions 91 to 109 of SEQ ID NO:10, at least one amino acid from positions 147 to 167 of SEQ ID NO:11, or at least one amino acid from positions 1016 to 1037 of SEQ ID NO:12, respectively, and wherein the peptide has only 2 or 3 amino acids from the wild-type amino acid sequence of KIAA2018 (SEQ ID NO:8); and
a peptide capable of inducing an immune response against a SLC22A9-1a frameshift mutant protein, wherein the peptide comprises an immunogenic fragment of any one of SEQ ID NOs:14-18 comprising at least 8 consecutive amino acids thereof, including at least one amino acid from positions 327 to 400 of SEQ ID NO:14, at least one amino acid from positions 335 to 400 of SEQ ID NO:15, at least one amino acid from positions 533 to 549 of SEQ ID NO:16, at least one amino acid from positions 327 to 400 of SEQ ID NO:17, or at least one amino acid from positions 335 to 400 of SEQ ID NO:18, respectively, and wherein the peptide comprises at least 18 amino acids and no more than 28 amino acids.

25. A peptide according to claim 24, wherein the peptide comprises a glycine residue at its C-terminus.

26. A peptide mixture comprising a first peptide and a second peptide,
wherein the first peptide is a peptide according to claim 24 and the second peptide is independently selected from (A) a peptide according to claim 24 different from said first peptide and (B) a peptide capable of inducing an immune response against a TGFβR2-1a frameshift mutant protein and comprising an immunogenic fragment selected from (i) an immunogenic fragment of SEQ ID NO; 3, wherein the fragment comprises at least 8 consecutive amino acids of SEQ ID NO:3 including at least one of positions 121 and 135 of SEQ ID NO; 3, and (ii) an immunogenic fragment of SEQ ID NO; 2, wherein the fragment comprises at least 10 consecutive amino acids of SEQ ID NO:19,
wherein the first peptide is capable of inducing an immune response against a different frameshift mutant protein from the second peptide.

27. A T-cell or a T-cell receptor or antigen-binding fragment thereof, specific for a peptide capable of inducing an immune response against a protein selected from:
a ASTE1-1a frameshift mutant protein, wherein the peptide comprises an immunogenic fragment of SEQ ID NO:5, wherein the fragment comprises at least 10 consecutive amino acids of SEQ ID NO:26, and wherein the peptide comprises at least 22 amino acids and no more than 31 amino acids;
a TAF1β-1a frameshift mutant protein, wherein the peptide comprises an immunogenic fragment of SEQ ID NO:7, wherein the fragment comprises at least 10 consecutive amino acids of SEQ ID NO:27, and wherein the peptide comprises no more than 35 amino acids;
a KIAA2018-1a frameshift mutant protein, wherein the peptide comprises a immunogenic fragment of one of SEQ ID NOs:9-12 comprising at least 8 consecutive amino acids thereof, including at least one amino acid from positions 13 to 37 of SEQ ID NO:9, at least one amino acid from positions 91 to 109 of SEQ ID NO:10, at least one amino acid from positions 147 to 167 of SEQ ID NO:11, or at least one amino acid from positions 1016 to 1037 of SEQ ID NO:12, respectively, and wherein the peptide has only 2 or 3 amino acids from the wild-type amino acid sequence of KIAA2018 (SEQ ID NO:8); or
a SLC22A9-1a frameshift mutant protein, wherein the peptide comprises an immunogenic fragment of one of SEQ ID NO:14-18 comprising at least 8 consecutive amino acids thereof, including at least one amino acid from positions 327 to 400 of SEQ ID NO:14, at least one amino acid from positions 335 to 400 of SEQ ID NO:15, at least one amino acid from positions 533 to 549 of SEQ ID NO:16, at least one amino acid from positions 327 to 400 of SEQ ID NO:17, or at least one amino acid from positions 335 to 400 of SEQ ID NO:18, respectively, and wherein the peptide comprises at least 18 amino acids and no more than 28 amino acids.

28. At least one nucleic acid molecule, wherein the or each nucleic acid molecule comprises a nucleotide sequence encoding at least one peptide according to claim 24.

29. A vector comprising the nucleic acid molecule according to claim 28.

30. A host cell comprising the vector according to claim 29.

31. A pharmaceutical composition comprising a peptide according to claim 24, and a pharmaceutically-acceptable carrier, diluent or excipient.

32. A method of treating and/or preventing cancer, comprising administering to a subject in need thereof a pharmaceutical composition comprising a peptide selected from:
a peptide capable of inducing an immune response against a ASTE1-1a frameshift mutant protein, wherein the peptide comprises an immunogenic fragment of SEQ ID NO:5, wherein the fragment comprises at least 10 consecutive amino acids of SEQ ID NO:26;
a peptide capable of inducing an immune response against a TAF1β-1a frameshift mutant protein, wherein the peptide comprises an immunogenic fragment of SEQ ID NO:7, wherein the fragment comprises at least 12 consecutive amino acids of SEQ ID NO:27, and wherein the peptide comprises no more than 27 amino acids;
a peptide capable of inducing an immune response against a KIAA2018-1a frameshift mutant protein, wherein the peptide comprises a immunogenic fragment of one of SEQ ID NOs: 9-12 comprising at least 8 consecutive amino acids thereof, including at least one amino acid from positions 13 to 37 of SEQ ID NO:9, at least one amino acid from positions 91 to 109 of SEQ ID NO:10, at least one amino acid from positions 147 to 167 of SEQ ID NO:11, or at least one amino acid from positions 1016 to 1037 of SEQ ID NO:12, respectively; and
a peptide capable of inducing an immune response against a SLC22A9-1a frameshift mutant protein, wherein the peptide comprises an immunogenic fragment of one of SEQ ID NO:14-18 comprising at least 8 consecutive amino acids thereof, including at least one amino acid from positions 327 to 400 of SEQ ID NO:14, at least one amino acid from positions 335 to 400 of SEQ ID NO:15, at least one amino acid from positions 533 to 549 of SEQ ID NO:16, at least one amino acid from positions 327 to 400 of SEQ ID NO:17, or at least one amino acid from positions 335 to 400 of SEQ ID NO:18, respectively,
or administering to a patient in need thereof a T-cell or a T-cell receptor or antigen-binding fragment thereof, specific for the peptide, a nucleic acid molecule encoding the peptide, a vector comprising the nucleic acid molecule, a host cell comprising the vector, or a pharmaceutical composition comprising the peptide, T-cell, T-cell receptor or antigen-binding fragment thereof, nucleic acid molecule, vector or host cell, preferably wherein the cancer is colorectal cancer.

33. A method of selecting a peptide mixture, peptide, nucleic acid molecule, vector, host cell, T-cell, T-cell receptor or antigen-binding fragment thereof, or a pharmaceutical composition for administration to a patient, comprising:
(i) identifying whether a cancer patient has a frameshift mutation in one or more of the ASTE1 protein, TAF1β protein, KIAA2018 protein and SLC22A9 protein and, if so,
(ii) selecting a peptide from:
a peptide capable of inducing an immune response against a ASTE1-1a frameshift mutant protein, wherein the peptide comprises an immunogenic fragment of SEQ ID NO:5, wherein the fragment comprises at least 10 consecutive amino acids of SEQ ID NO:26;
a peptide capable of inducing an immune response against a TAF1β-1a frameshift mutant protein, wherein the peptide comprises an immunogenic fragment of SEQ ID NO:7, wherein the fragment comprises at least 12 consecutive amino acids of SEQ ID NO:27, and wherein the peptide comprises no more than 27 amino acids;
a peptide capable of inducing an immune response against a KIAA2018-1a frameshift mutant protein, wherein the peptide comprises a immunogenic fragment of one of SEQ ID NOs:9-12 comprising at least 8 consecutive amino acids thereof, including at least one amino acid from positions 13 to 37 of SEQ ID NO:9, at least one amino acid from positions 91 to 109 of SEQ ID NO:10, at least one amino acid from positions 147 to 167 of SEQ ID NO:11, or at least one amino acid from positions 1016 to 1037 of SEQ ID NO:12, respectively; and
a peptide capable of inducing an immune response against a SLC22A9-1a frameshift mutant protein, wherein the peptide comprises an immunogenic fragment of one of SEQ ID NO:14-18 comprising at least 8 consecutive amino acids thereof, including at least one amino acid from positions 327 to 400 of SEQ ID NO:14, at least one amino acid from positions 335 to 400 of SEQ ID NO:15, at least one amino acid from positions 533 to 549 of SEQ ID NO:16, at least one amino acid from positions 327 to 400 of SEQ ID NO:17, or at least one amino acid from positions 335 to 400 of SEQ ID NO:18, respectively,
or selecting a T-cell or a T-cell receptor or an antigen-binding fragment thereof, specific for the peptide, a nucleic acid molecule encoding the peptide, a vector comprising the nucleic acid molecule, a host cell comprising the vector, or a pharmaceutical composition comprising the peptide, nucleic acid molecule, T-cell, T-cell receptor or antigen-binding fragment thereof, vector or host cell.


SEQUENCE LISTING (129 sequences)

SEQ ID NO: 1 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 2 - protein, 161 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKSLVRLSSCVPVALMSAMTTSSSQKNITPAILTCC

SEQ ID NO: 3 - protein, 161 aa, Artificial Sequence
  Modified peptide
  VARIANT 121: Any amino acid except cysteine
  VARIANT 135: Any amino acid except cysteine
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKXIMKEKKSLVRLSSXVPVALMSAMTTSSSQKNITPAILTCC

SEQ ID NO: 4 - protein, 679 aa, Homo sapiens
MGIRGLMSFVEDHSNEFFTDLKLRDTKIVIDGYALFHRLCFSSNLDLRYGGDYDSFADVVQKFFESLFACNICPYVVLDGGCDISDKKLTTLKDRAREKIQMAHSLSVGGSGYVCPLLIREVFIQVLIKLRVCFVQCFSEADRDIMTLANHWNCPVLSSDSDFCIFDLKTGFCPLNSFQWRNMNTIKGTQNYIPAKCFSLDAFCHHFSNMNKALLPLFAVLCGNDHVNLPIMETFLSKARLPLGATSSKGRRHHRILGLLNWLSHFANPTEALDNVLKYLPKKDRENVKELLCCSMEEYQQSQVKLQDFFQCGTYVCPDALNLGLPEWVLVALAKGQLSPFISDALVLRRTILPTQVENMQQPNAHRISQPIRQIIYGLLLNASPHLDKTSWNALPPQPLAFSEVERINKNIRTSIIDAVELAKDHSDLSRLTELSLRRRQMLLLETLKVKQTILEPIPTSLKLPIAVSCYWLQHTETKAKLHHLQSLLLTMLVGPLIAIINSPGKEELQEDGAKMLYAEFQRVKAQTRLGTRLDLDTAHIFCQWQSCLQMGMYLNQLLSTPLPEPDLTRLYSGSLVHGLCQQLLASTSVESLLSICPEAKQLYEYLFNATRSYAPAEIFLPKGRSNSKKKRQKKQNTSCSKNRGRTTAHTKCWYEGNNRFGLLMVENLEEHSEASNIE

SEQ ID NO: 5 - protein, 663 aa, Homo sapiens
MGIRGLMSFVEDHSNEFFTDLKLRDTKIVIDGYALFHRLCFSSNLDLRYGGDYDSFADVVQKFFESLFACNICPYVVLDGGCDISDKKLTTLKDRAREKIQMAHSLSVGGSGYVCPLLIREVFIQVLIKLRVCFVQCFSEADRDIMTLANHWNCPVLSSDSDFCIFDLKTGFCPLNSFQWRNMNTIKGTQNYIPAKCFSLDAFCHHFSNMNKALLPLFAVLCGNDHVNLPIMETFLSKARLPLGATSSKGRRHHRILGLLNWLSHFANPTEALDNVLKYLPKKDRENVKELLCCSMEEYQQSQVKLQDFFQCGTYVCPDALNLGLPEWVLVALAKGQLSPFISDALVLRRTILPTQVENMQQPNAHRISQPIRQIIYGLLLNASPHLDKTSWNALPPQPLAFSEVERINKNIRTSIIDAVELAKDHSDLSRLTELSLRRRQMLLLETLKVKQTILEPIPTSLKLPIAVSCYWLQHTETKAKLHHLQSLLLTMLVGPLIAIINSPGKEELQEDGAKMLYAEFQRVKAQTRLGTRLDLDTAHIFCQWQSCLQMGMYLNQLLSTPLPEPDLTRLYSGSLVHGLCQQLLASTSVESLLSICPEAKQLYEYLFNATRSYAPAEIFLPKGRSNSKKKGRRNRIPAVLRTEGEPLHTPSVGMRETTGLGC

SEQ ID NO: 6 - protein, 587 aa, Homo sapiens
MDLEEAEEFKERCTQCAAVSWGLTDEGKYYCTSCHNVTERYQEVTNTDLIPNTQIKALNRGLKKKNTEKGWDWYVCEGFQYILYQQAEALKNLGVGPELKNDVLHNFWKRYLQKSKQAYCKNPVYTTGRKPTVLEDNLSHSDWASEPELLSDVSCPPFLESGAESQSDIHTRKPFPVSKASQSETSVCSGSLDGVEYSQRKEKGIVKMTMPQTLAFCYLSLLWQREAITLSDLLRFVEEDHIPYINAFQHFPEQMKLYGRDRGIFGIESWPDYEDIYKKTVEVGTFLDLPRFPDITEDCYLHPNILCMKYLMEVNLPDEMHSLTCHVVKMTGMGEVDFLTFDPIAKMAKTVKYDVQAVAIIVVVLKLLFLLDDSFEWSLSNLAEKHNEKNKKDKPWFDFRKWYQIMKKAFDEKKQKWEEARAKYLWKSEKPLYYSFVDKPVAYKKREMVVNLQKQFSTLVESTATAGKKSPSSFQFNWTEEDTDRTCFHGHSLQGVLKEKGQSLLTKNSLYWLSTQKFCRCYCTHVTTYEESNYSLSYQFILNLFSFLLRIKTSLLHEEVSLVEKKLFEKKYSVKRKKSRSKKVRRH

SEQ ID NO: 7 - protein, 90 aa, Homo sapiens
MDLEEAEEFKERCTQCAAVSWGLTDEGKYYCTSCHNVTERYQEVTNTDLIPNTQIKALNRGLKKKTILKKAGIGMCVKVSSIFFINKQKP

SEQ ID NO: 8 - protein, 2245 aa, Homo sapiens
MPEMTENETPTKKQHRKKNRETHNAVERHRKKKINAGINRIGELIPCSPALKQSKNMILDQAFKYITELKRQNDELLLNGGNNEQAEEIKKLRKQLEEIQKENGRYIELLKANDICLYDDPTIHWKGNLKNSKVSVVIPSDQVQKKIIVYSNGNQPGGNSQGTAVQGITFNVSHNLQKQTANVVPVQRTCNLVTPVSISGVYPSENKPWHQTTVPALATNQPVPLCLPAAISAQSILELPTSESESNVLGATSGSLIAVSIESEPHQHHSLHTCLNDQNSSENKNGQENPKVLKKMTPCVTNIPHSSSATATKVHHGNKSCLSIQDFRGDFQNTFVVSVTTTVCSQPPRTAGDSSPMSISKSADLTSTATVVASSAPGVGKATIPISTLSGNPLDNGWTLSCSLPSSSVSTSDLKNINSLTRISSAGNTQTTWTTLQLAGNTIQPLSQTPSSAVTPVLNESGTSPTTSNHSRYVATDINLNNSFPADGQPVEQVVVTLPSCPSLPMQPLIAQPQVKSQPPKNILPLNSAMQVIQMAQPVGSAVNSAPTNQNVIILQPPSTTPCPTVMRAEVSNQTVGQQIVIIQAANQNPLPLLPAPPPGSVRLPINGANTVIGSNNSVQNVPTPQTFGGKHLVHILPRPSSLSASNSTQTFSVTMSNQQPQTISLNGQLFALQPVMSSSGTTNQTPMQIIQPTTSEDPNTNVALNTFGALASLNQSISQMAGQSCVQLSISQPANSQTAANSQTTTANCVSLTTTAAPPVTTDSSATLASTYNLVSTSSMNTVACLPNMKSKRLNKKPGGRKHLAANKSACPLNSVRDVSKLDCPNTEGSAEPPCNDGLLESFPAVLPSVSVSQANSVSVSASHSLGVLSSESLIPESVSKSKSAEKSSPPSQESVTSEHFAMAAAKSKDSTPNLQQETSQDKPPSSLALSDAAKPCASANVLIPSPSDPHILVSQVPGLSSTTSTTSTDCVSEVEIIAEPCRVEQDSSDTMQTTGLLKGQGLTTLLSDLAKKKNPQKSSLSDQMDHPDFSSENPKIVDSSVNLHPKQELLLMNNDDRDPPQHHSCLPDQEVINGSLINGRQADSPMSTSSGSSRSFSVASMLPETTREDVTSNATTNTCDSCTFVEQTDIVALAARAIFDQENLEKGRVGLQADIREVASKPSEASLLEGDPPFKSQIPKESGTGQAEATPNEFNSQGSIEATMERPLEKPSCSLGIKTSNASLQDSTSQPPSITSLSVNNLIHQSSISHPLASCAGLSPTSEQTTVPATVNLTVSSSSYGSQPPGPSLMTEYSQEQLNTMTSTIPNSQIQEPLLKPSHESRKDSAKRAVQDDLLLSSAKRQKHCQPAPLRLESMSLMSRTPDTISDQTQMMVSQIPPNSSNSVVPVSNPAHGDGLTRLFPPSNNFVTPALRQTEVQCGSQPSVAEQQQTQASQHLQALQQHVPAQGVSHLHSNHLYIKQQQQQQQQQQQQQQQQQAGQLRERHHLYQMQHHVPHAESSVHSQPHNVHQQRTLQQEVQMQKKRNLVQGTQTSQLSLQPKHHGTDQSRSKTGQPHPHHQQMQQQMQQHFGSSQTEKSCENPSTSRNHHNHPQNHLNQDIMHQQQDVGSRQQGSGVSSEHVSGHNPMQRLLTSRGLEQQMVSQPSIVTRSSDMTCTPHRPERNRVSSYSAEALIGKTSSNSEQRMGISIQGSRVSDQLEMRSYLDVPRNKSLAIHNMQGRVDHTVASDIRLSDCQTFKPSGASQQPQSNFEVQSSRNNEIGNPVSSLRSMQSQAFRISQNTGPPPIDRQKRLSYPPVQSIPTGNGIPSRDSENTCHQSFMQSLLAPHLSDQVIGSQRSLSEHQRNTQCGPSSAIEYNCPPTHENVHIRRESESQNRESCDMSLGAINTRNSTLNIPFSSSSSSGDIQGRNTSPNVSVQKSNPMRITESHATKGHMNPPVTTNMHGVARPALPHPSVSHGNGDQGPAVRQANSSVPQRSRHPLQDSSGSKIRQPERNRSGNQRQSTVFDPSLPHLPLSTGGSMILGRQQPATEKRGSIVRFMPDSPQVPNDNSGPDQHTLSQNFGFSFIPEGGMNPPINANASFIPQVTQPSATRTPALIPVDPQNTLPSFYPPYSPAHPTLSNDISIPYFPNQMFSNPSTEKVNSGSLNNRFGSILSPPRPVGFAQPSFPLLPDMPPMHMTNSHLSNFNMTSLFPEIATALPDGSAMSPLLTIANSSASDSSKQSSNRPAHNISHILGHDCSSAV

SEQ ID NO: 9 - protein, 37 aa, Homo sapiens
MPEMTENETPTKSSTERKTGRHTMQWRGIERRKLMLG

SEQ ID NO: 10 - protein, 109 aa, Homo sapiens
MPEMTENETPTKKQHRKKNRETHNAVERHRKKKINAGINRIGELIPCSPALKQSKNMILDQAFKYITELKRQNDELLLNGGNNEQAEEIKSYGNNWKKSKKKMADILNY

SEQ ID NO: 11 - protein, 167 aa, Homo sapiens [sequence omitted]

SEQ ID NO: 12 - protein, 1037 aa, Homo sapiens [sequence omitted]

SEQ ID NO: 13 - protein, 553 aa, Homo sapiens
MAFQDLLGHAGDLWRFQILQTVFLSIFAVATYLHFMLENFTAFIPGHRCWVHILDNDTVSDNDTGALSQDALLRISIPLDSNMRPEKCRRFVHPQWQLLHLNGTFPNTSDADMEPCVDGWVYDRISFSSTIVTEWDLVCDSQSLTSVAKFVFMAGMMVGGILGGHLSDRFGRRFVLRWCYLQVAIVGTCAALAPTFLIYCSLRFLSGIAAMSLITNTIMLIAEWATHRFQAMGITLGMCPSGIAFMTLAGLAFAIRDWHILQLVVSVPYFVIFLTSSWLLESARWLIINNKPEEGLKELRKAAHRSGMKNARDTLTLEILKSTMKKELEAAQKKKPSLCEMLHMPNICKRISLLSFTRFANFMAYFGLNLHVQHLGNNVFLLQTLFGAVILLANCVAPWALKYMNRRASQMLLMIFLLACLLAIIFVPQEMQTLREVLATLGLGASALANTLAFAHGNEVIPTIIRARAMGINATFANIAGALAPLMMILSVYSPPLPWIIYGVFPFISGFAFLLLPETRNKPLFDTIQDEKNERKDPREPKQEDPRVEVTQF

SEQ ID NO: 14 - protein, 400 aa, Homo sapiens
MAFQDLLGHAGDLWRFQILQTVFLSIFAVATYLHFMLENFTAFIPGHRCWVHILDNDTVSDNDTGALSQDALLRISIPLDSNMRPEKCRRFVHPQWQLLHLNGTFPNTSDADMEPCVDGWVYDRISFSSTIVTEWDLVCDSQSLTSVAKFVFMAGMMVGGILGGHLSDRFGRRFVLRWCYLQVAIVGTCAALAPTFLIYCSLRFLSGIAAMSLITNTIMLIAEWATHRFQAMGITLGMCPSGIAFMTLAGLAFAIRDWHILQLVVSVPYFVIFLTSSWLLESARWLIINNKPEEGLKELRKAAHRSGMKNARDTLTLEILKSTMKKNWRQHKKKNLLCVKCSTCPTYVKGSPSCPLRDLQTLWPILALISMSSIWGTMFSCCRLSLVQSSSWPTVLHLGH

SEQ ID NO: 15 - protein, 400 aa, Homo sapiens [sequence omitted]

SEQ ID NO: 16 - protein, 549 aa, Homo sapiens
MAFQDLLGHAGDLWRFQILQTVFLSIFAVATYLHFMLENFTAFIPGHRCWVHILDNDTVSDNDTGALSQDALLRISIPLDSNMRPEKCRRFVHPQWQLLHLNGTFPNTSDADMEPCVDGWVYDRISFSSTIVTEWDLVCDSQSLTSVAKFVFMAGMMVGGILGGHLSDRFGRRFVLRWCYLQVAIVGTCAALAPTFLIYCSLRFLSGIAAMSLITNTIMLIAEWATHRFQAMGITLGMCPSGIAFMTLAGLAFAIRDWHILQLVVSVPYFVIFLTSSWLLESARWLIINNKPEEGLKELRKAAHRSGMKNARDTLTLEILKSTMKKELEAAQKKKPSLCEMLHMPNICKRISLLSFTRFANFMAYFGLNLHVQHLGNNVFLLQTLFGAVILLANCVAPWALKYMNRRASQMLLMFLLAICLLAIIFVPQEMQTLREVLATLGLGASALANTLAFAHGNEVIPTIIRARAMGINATFANIAGALAPLMMILSVYSPPLPWIIYGVFPFISGFAFLLLPETRNKPLFDTIQDEKMREKTPENQSKRIREWK

SEQ ID NO: 17 - protein, 400 aa, Artificial Sequence
  Modified peptide
  VARIANT 354: Any amino acid except cysteine
MAFQDLLGHAGDLWRFQILQTVFLSIFAVATYLHFMLENFTAFIPGHRCWVHILDNDTVSDNDTGALSQDALLRISIPLDSNMRPEKCRRFVHPQWQLLHLNGTFPNTSDADMEPCVDGWVYDRISFSSTIVTEWDLVCDSQSLTSVAKFVFMAGMMVGGILGGHLSDRFGRRFVLRWCYLQVAIVGTCAALAPTFLIYCSLRFLSGIAAMSLITNTIMLIAEWATHRFQAMGITLGMCPSGIAFMTLAGLAFAIRDWHILQLVVSVPYFVIFLTSSWLLESARWLIINNKPEEGLKELRKAAHRSGMKNARDTLTLEILKSTMKKNWRQHKKKNLLCVKCSTCPTYVKGSPSXPLRDLQTLWPILALISMSSIWGTMFSCCRLSLVQSSSWPTVLHLGH

SEQ ID NO: 18 - protein, 400 aa, Artificial Sequence
  Modified peptide
  VARIANT 344: Any amino acid except cysteine
MAFQDLLGHAGDLWRFQILQTVFLSIFAVATYLHFMLENFTAFIPGHRCWVHILDNDTVSDNDTGALSQDALLRISIPLDSNMRPEKCRRFVHPQWQLLHLNGTFPNTSDADMEPCVDGWVYDRISFSSTIVTEWDLVCDSQSLTSVAKFVFMAGMMVGGILGGHLSDRFGRRFVLRWCYLQVAIVGTCAALAPTFLIYCSLRFLSGIAAMSLITNTIMLIAEWATHRFQAMGITLGMCPSGIAFMTLAGLAFAIRDWHILQLVVSVPYFVIFLTSSWLLESARWLIINNKPEEGLKELRKAAHRSGMKNARDTLTLEILKSTMKKELEAAQKKNLLCVKCSTXPTYVKGSPSCPLRDLQTLWPILALISMSSIWGTMFSCCRLSLVQSSSWPTVLHLGH

SEQ ID NO: 19 - protein, 33 aa, Homo sapiens
KCIMKEKKSLVRLSSCVPVALMSAMTTSSSQKN

SEQ ID NO: 20 - protein, 24 aa, Homo sapiens
KCIMKEKKSLVRLSSCVPVALMSA

SEQ ID NO: 21 - protein, 24 aa, Artificial Sequence
  Modified peptide
KGIMKEKKSLVRLSSCVPVALMSA

SEQ ID NO: 22 - protein, 20 aa, Homo sapiens
SSCVPVALMSAMTTSSSQKN

SEQ ID NO: 23 - protein, 20 aa, Artificial Sequence
  Modified peptide
SSGVPVALMSAMTTSSSQKN

SEQ ID NO: 24 - protein, 17 aa, Homo sapiens
KCIMKEKKSLVRLSSCV

SEQ ID NO: 25 - protein, 17 aa, Artificial Sequence
  Modified peptide
KGIMKEKKSLVRLSSCV

SEQ ID NO: 26 - protein, 25 aa, Homo sapiens
RNRIPAVLRTEGEPLHTPSVGMRET

SEQ ID NO: 27 - protein, 25 aa, Homo sapiens
KTILKKAGIGMCVKVSSIFFINKQK

SEQ ID NO: 28 - protein, 24 aa, Homo sapiens
KKTLRNHLFLIRWIILTFLQKILK

SEQ ID NO: 29 - protein, 28 aa, Homo sapiens
SCPLRDLQTLWPILALISMSSIWGTMFS

SEQ ID NO: 30 - protein, 28 aa, Artificial Sequence
  Modified peptide
SGPLRDLQTLWPILALISMSSIWGTMFS

SEQ ID NO: 31 - protein, 19 aa, Homo sapiens
STCPTYVKGSPSCPLRDLQ

SEQ ID NO: 32 - protein, 19 aa, Artificial Sequence
  Modified peptide
STGPTYVKGSPSCPLRDLQ

SEQ ID NO: 33 - protein, 9 aa, Homo sapiens
RLSSCVPVA

SEQ ID NO: 34 - protein, 23 aa, Homo sapiens
SLVRLSSCVPVALMSAMTTSSSQ

SEQ ID NO: 35 - protein, 22 aa, Homo sapiens
SPKCIMKEKKSLVRLSSCVPVA

SEQ ID NO: 36 - protein, 19 aa, Homo sapiens
PKCIMKEKKKSLVRLSSCV

SEQ ID NO: 37 - protein, 9 aa, Homo sapiens
SLVRLSSCV

SEQ ID NO: 38 - protein, 23 aa, Homo sapiens
ALMSAMTTSSSQKNITPAILTCC

SEQ ID NO: 39 - protein, 19 aa, Homo sapiens
AMTTSSSQKNITPAILTCC

SEQ ID NO: 40 - protein, 33 aa, Homo sapiens
SPKCIMKEKKSLVRLSSCVPVALMSAMTTSSSQ

SEQ ID NO: 41 - protein, 15 aa, Homo sapiens
KKSLVRLSSCVPVAL

SEQ ID NO: 42 - protein, 15 aa, Homo sapiens
VALMSAMTTSSSQKN

SEQ ID NO: 43 - protein, 15 aa, Homo sapiens
PVALMSAMTTSSSQK

SEQ ID NO: 44 - protein, 15 aa, Homo sapiens
KCIMKEKKSLVRLSS

SEQ ID NO: 45 - protein, 15 aa, Homo sapiens
CVPVALMSAMTTSSS

SEQ ID NO: 46 - protein, 15 aa, Homo sapiens
LVRLSSCVPVALMSA

SEQ ID NO: 47 - protein, 15 aa, Homo sapiens
MKEKKSLVRLSSCVP

SEQ ID NO: 48 - protein, 15 aa, Homo sapiens
KSLVRLSSCVPVALM

SEQ ID NO: 49 - protein, 15 aa, Homo sapiens
SSCVPVALMSAMTTS

SEQ ID NO: 50 - protein, 40 aa, Homo sapiens
GRSNSKKKGRRNRIPAVLRTEGEPLHTPSVGMRETTGLGC

SEQ ID NO: 51 - protein, 15 aa, Homo sapiens
EGEPLHTPSVGMRET

SEQ ID NO: 52 - protein, 15 aa, Homo sapiens
RTEGEPLHTPSVGMR

SEQ ID NO: 53 - protein, 15 aa, Homo sapiens
IPAVLRTEGEPLHTP

SEQ ID NO: 54 - protein, 15 aa, Homo sapiens
RNRIPAVLRTEGEPL

SEQ ID NO: 55 - protein, 25 aa, Homo sapiens
RNRIPAVLRTEGEPLHTPSVGMRET

SEQ ID NO: 56 - protein, 8 aa, Homo sapiens
TEGEPLHT

SEQ ID NO: 57 - protein, 9 aa, Homo sapiens
LRTEGEPLH

SEQ ID NO: 58 - protein, 9 aa, Homo sapiens
VLRTEGEPL

SEQ ID NO: 59 - protein, 37 aa, Homo sapiens
QIKALNRGLKKKTILKKAGIGMCVKVSSIFFINKQKP

SEQ ID NO: 60 - protein, 15 aa, Homo sapiens
CVKVSSIFFINKQKP

SEQ ID NO: 61 - protein, 15 aa, Homo sapiens
GMCVKVSSIFFINKQ

SEQ ID NO: 62 - protein, 15 aa, Homo sapiens
IGMCVKVSSIFFINK

SEQ ID NO: 63 - protein, 15 aa, Homo sapiens
GIGMCVKVSSIFFIN

SEQ ID NO: 64 - protein, 15 aa, Homo sapiens
KAGIGMCVKVSSIFF

SEQ ID NO: 65 - protein, 15 aa, Homo sapiens
KKTILKKAGIGMCVK

SEQ ID NO: 66 - protein, 15 aa, Homo sapiens
LKKKTILKKAGIGMC

SEQ ID NO: 67 - protein, 15 aa, Homo sapiens
GLKKKTILKKAGIGM

SEQ ID NO: 68 - protein, 15 aa, Homo sapiens
NRGLKKKTILKKAGI

SEQ ID NO: 69 - protein, 32 aa, Homo sapiens
NRGLKKKTILKKAGIGMCVKVSSIFFINKQKP

SEQ ID NO: 70 - protein, 9 aa, Homo sapiens
SIFFINKQK

SEQ ID NO: 71 - protein, 9 aa, Homo sapiens
VSSIFFINK

SEQ ID NO: 72 - protein, 9 aa, Homo sapiens
GMCVKVSSI

SEQ ID NO: 73 - protein, 9 aa, Homo sapiens
AGIGMCVKV

SEQ ID NO: 74 - protein, 9 aa, Homo sapiens
KAGIGMCVK

SEQ ID NO: 75 - protein, 9 aa, Homo sapiens
KTILKKAGI

SEQ ID NO: 76 - protein, 32 aa, Homo sapiens
TLLSDLAKKKTLRNHLFLIRWIILTFLQKILK

SEQ ID NO: 77 - protein, 15 aa, Homo sapiens
LIRWIILTFLQKILK

SEQ ID NO: 78 - protein, 15 aa, Homo sapiens
LFLIRWIILTFLQKI

SEQ ID NO: 79 - protein, 14 aa, Homo sapiens
HLFLIRWIILTFLQ

SEQ ID NO: 80 - protein, 15 aa, Homo sapiens
RNHLFLIRWIILTFL

SEQ ID NO: 81 - protein, 15 aa, Homo sapiens
KKTLRNHLFLIRWII

SEQ ID NO: 82 - protein, 24 aa, Homo sapiens
KKTLRNHLFLIRWIILTFLQKILK

SEQ ID NO: 83 - protein, 9 aa, Homo sapiens
LTFLQKILK

SEQ ID NO: 84 - protein, 9 aa, Homo sapiens
ILTFLQKIL

SEQ ID NO: 85 - protein, 9 aa, Homo sapiens
IILTFLQKI

SEQ ID NO: 86 - protein, 9 aa, Homo sapiens
WIILTFLQK

SEQ ID NO: 87 - protein, 9 aa, Homo sapiens
IRWIILTFL

SEQ ID NO: 88 - protein, 9 aa, Homo sapiens
LIRWIILTF

SEQ ID NO: 89 - protein, 9 aa, Homo sapiens
FLIRWIILT

SEQ ID NO: 90 - protein, 9 aa, Homo sapiens
LFLIRWIIL

SEQ ID NO: 91 - protein, 9 aa, Homo sapiens
NHLFLIRWI

SEQ ID NO: 92 - protein, 9 aa, Homo sapiens
LRNHLFLIR

SEQ ID NO: 93 - protein, 9 aa, Homo sapiens
TLRNHLFLI

SEQ ID NO: 94 - protein, 9 aa, Homo sapiens
KTLRNHLFL

SEQ ID NO: 95 - protein, 9 aa, Homo sapiens
KKTLRNHLF

SEQ ID NO: 96 - protein, 68 aa, Homo sapiens
KKNLLCVKCSTCPTYVKGSPSCPLRDLQTLWPILALISMSSIWGTMFSCCRLSLVQSSSWPTVLHLGH

SEQ ID NO: 97 - protein, 15 aa, Homo sapiens
WGTMFSCCRLSLVQS

SEQ ID NO: 98 - protein, 15 aa, Homo sapiens
ISMSSIWGTMFSCCR

SEQ ID NO: 99 - protein, 15 aa, Homo sapiens
LISMSSIWGTMFSCC

SEQ ID NO: 100 - protein, 15 aa, Homo sapiens
ILALISMSSIWGTMF

SEQ ID NO: 101 - protein, 15 aa, Homo sapiens
WPILALISMSSIWGT

SEQ ID NO: 102 - protein, 15 aa, Homo sapiens
LWPILALISMSSIWG

SEQ ID NO: 103 - protein, 15 aa, Homo sapiens
QTLWPILALISMSSI

SEQ ID NO: 104 - protein, 15 aa, Homo sapiens
LQTLWPILALISMSS

SEQ ID NO: 105 - protein, 15 aa, Homo sapiens
LRDLQTLWPILALIS

SEQ ID NO: 106 - protein, 15 aa, Homo sapiens
SCPLRDLQTLWPILA

SEQ ID NO: 107 - protein, 15 aa, Homo sapiens
PTYVKGSPSCPLRDL

SEQ ID NO: 108 - protein, 15 aa, Homo sapiens
CPTYVKGSPSCPLRD

SEQ ID NO: 109 - protein, 15 aa, Homo sapiens
STCPTYVKGSPSCPL

SEQ ID NO: 110 - protein, 15 aa, Homo sapiens
KNLLCVKCSTCPTYV

SEQ ID NO: 111 - protein, 28 aa, Homo sapiens
SCPLRDLQTLWPILALISMSSIWGTMFS

SEQ ID NO: 112 - protein, 19 aa, Homo sapiens
STCPTYVKGSPSCPLRDLQ

SEQ ID NO: 113 - protein, 9 aa, Homo sapiens
LALISMSSI

SEQ ID NO: 114 - protein, 9 aa, Homo sapiens
TLWPILALI

SEQ ID NO: 115 - protein, 9 aa, Homo sapiens
QTLWPILAL

SEQ ID NO: 116 - protein, 9 aa, Homo sapiens
CPLRDLQTL

SEQ ID NO: 117 - protein, 9 aa, Homo sapiens
SPSCPLRDL

SEQ ID NO: 118 - protein, 9 aa, Homo sapiens
CSTCPTYVK

SEQ ID NO: 119 - protein, 9 aa, Homo sapiens
SSWPTVLHL

SEQ ID NO: 120 - protein, 9 aa, Homo sapiens
LSLVQSSSW

SEQ ID NO: 121 - protein, 9 aa, Homo sapiens
RLSLVQSSS

SEQ ID NO: 122 - protein, 9 aa, Homo sapiens
FSCCRLSLV

SEQ ID NO: 123 - protein, 14 aa, Homo sapiens
VRLSSCVPVALMSA

SEQ ID NO: 124 - protein, 9 aa, Homo sapiens
LVRLSSCVP

SEQ ID NO: 125 - protein, 9 aa, Artificial Sequence
  Modified peptide
LVRLSSGVP

SEQ ID NO: 126 - protein, 27 aa, Artificial Sequence
  Modified peptide
KGIMKEKKSLVRLSSCVPVALMSAMTG

SEQ ID NO: 127 - protein, 31 aa, Homo sapiens
KKKGRRNRIPAVLRTEGEPLHTPSVGMRETG

SEQ ID NO: 128 - protein, 30 aa, Homo sapiens
GLKKKTILKKAGIGMCVKVSSIFFINKQKG

SEQ ID NO: 129 - protein, 27 aa, Homo sapiens
KCIMKEKKSLVRLSSCVPVALMSAMTG